SEQUENCE LISTING (14 sequences)

SEQ ID NO: 1 - DNA, 5480 bp, Homo sapiens
  source 1..5480: mol_type = genomic DNA
tatatccgtggtttcctgctacctccaaccatgggccttttgggaatactttgttttttaatcttcctggggaaaacctggggacaggagcaaacatatgtcatttcagcaccaaaaatattccgtgttggagcatctgaaaatattgtgattcaagtttatggatacactgaagcatttgatgcaacaatctctattaaaagttatcctgataaaaaatttagttactcctcaggccatgttcatttatcctcagagaataaattccaaaactctgcaatcttaacaatacaaccaaaacaattgcctggaggacaaaacccagtttcttatgtgtatttggaagttgtatcaaagcatttttcaaaatcaaaaagaatgccaataacctatgacaatggatttctcttcattcatacagacaaacctgtttatactccagaccagtcagtaaaagttagagtttattcgttgaatgacgacttgaagccagccaaaagagaaactgtcttaactttcatagatcctgaaggatcagaagttgacatggtagaagaaattgatcatattggaattatctcttttcctgacttcaagattccgtctaatcctagatatggtatgtggacgatcaaggctaaatataaagaggacttttcaacaactggaaccgcatattttgaagttaaagaatatgtcttgccacatttttctgtctcaatcgagccagaatataatttcattggttacaagaactttaagaattttgaaattactataaaagcaagatatttttataataaagtagtcactgaggctgacgtttatatcacatttggaataagagaagacttaaaagatgatcaaaaagaaatgatgcaaacagcaatgcaaaacacaatgttgataaatggaattgctcaagtcacatttgattctgaaacagcagtcaaagaactgtcatactacagtttagaagatttaaacaacaagtacctttatattgctgtaacagtcatagagtctacaggtggattttctgaagaggcagaaatacctggcatcaaatatgtcctctctccctacaaactgaatttggttgctactcctcttttcctgaagcctgggattccatatcccatcaaggtgcaggttaaagattcgcttgaccagttggtaggaggagtcccagtaacactgaatgcacaaacaattgatgtaaaccaagagacatctgacttggatccaagcaaaagtgtaacacgtgttgatgatggagtagcttcctttgtgcttaatctcccatctggagtgacggtgctggagtttaatgtcaaaactgatgctccagatcttccagaagaaaatcaggccagggaaggttaccgagcaatagcatactcatctctcagccaaagttacctttatattgattggactgataaccataaggctttgctagtgggagaacatctgaatattattgttacccccaaaagcccatatattgacaaaataactcactataattacttgattttatccaagggcaaaattatccactttggcacgagggagaaattttcagatgcatcttatcaaagtataaacattccagtaacacagaacatggttccttcatcccgacttctggtctattacatcgtcacaggagaacagacagcagaattagtgtctgattcagtctggttaaatattgaagaaaaatgtggcaaccagctccaggttcatctgtctcctgatgcagatgcatattctccaggccaaactgtgtctcttaatatggcaactggaatggattcctgggtggcattagcagcagtggacagtgctgtgtatggagtccaaagaggagccaaaaagcccttggaaagagtatttcaattcttagagaagagtgatctgggctgtggggcaggtggtggcctcaacaatgccaatgtgttccacctagctggacttaccttcctcactaatgcaaatgcagatgactcccaagaaaatgatgaaccttgtaaagaaattctcaggccaagaagaacgctgcaaaagaagatagaagaaatagctgctaaatataaacattcagtagtgaagaaatgttgttacgatggagcctgcgttaataatgatgaaacctgtgagcagcgagctgcacggattagtttagggccaagatgcatcaaagctttcactgaatgttgtgtcgtcgcaagccagctccgtgctaatatctctcataaagacatgcaattgggaaggctacacatgaagaccctgttaccagtaagcaagccagaaattcggagttattttccagaaagctggttgtgggaagttcatcttgttcccagaagaaaacagttgcagtttgccctacctgattctctaaccacctgggaaattcaaggcgttggcatttcaaacactggtatatgtgttgctgatactgtcaaggcaaaggtgttcaaagatgtcttcctggaaatgaatataccatattctgttgtacgaggagaacagatccaattgaaaggaactgtttacaactataggacttctgggatgcagttctgtgttaaaatgtctgctgtggagggaatctgcacttcggaaagcccagtcattgatcatcagggcacaaagtcctccaaatgtgtgcgccagaaagtagagggctcctccagtcacttggtgacattcactgtgcttcctctggaaattggccttcacaacatcaatttttcactggagacttggtttggaaaagaaatcttagtaaaaacattacgagtggtgccagaaggtgtcaaaagggaaagctattctggtgttactttggatcctaggggtatttatggtaccattagcagacgaaaggagttcccatacaggatacccttagatttggtccccaaaacagaaatcaaaaggattttgagtgtaaaaggactgcttgtaggtgagatcttgtctgcagttctaagtcaggaaggcatcaatatcctaacccacctccccaaagggagtgcagaggcggagctgatgagcgttgtcccagtattctatgtttttcactacctggaaacaggaaatcattggaacatttttcattctgacccattaattgaaaagcagaaactgaagaaaaaattaaaagaagggatgttgagcattatgtcctacagaaatgctgactactcttacagtgtgtggaagggtggaagtgctagcacttggttaacagcttttgctttaagagtacttggacaagtaaataaatacgtagagcagaaccaaaattcaatttgtaattctttattgtggctagttgagaattatcaattagataatggatctttcaaggaaaattcacagtatcaaccaataaaattacagggtaccttgcctgttgaagcccgagagaacagcttatatcttacagcctttactgtgattggaattagaaaggctttcgatatatgccccctggtgaaaatcgacacagctctaattaaagctgacaactttctgcttgaaaatacactgccagcccagagcacctttacattggccatttctgcgtatgctctttccctgggagataaaactcacccacagtttcgttcaattgtttcagctttgaagagagaagctttggttaaaggtaatccacccatttatcgtttttggaaagacaatcttcagcataaagacagctctgtacctaacactggtacggcacgtatggtagaaacaactgcctatgctttactcaccagtctgaacttgaaagatataaattatgttaacccagtcatcaaatggctatcagaagagcagaggtatggaggtggcttttattcaacccaggacacaatcaatgccattgagggcctgacggaatattcactcctggttaaacaactccgcttgagtatggacatcgatgtttcttacaagcataaaggtgccttacataattataaaatgacagacaagaatttccttgggaggccagtagaggtgcttctcaatgatgacctcattgtcagtacaggatttggcagtggcttggctacagtacatgtaacaactgtagttcacaaaaccagtacctctgaggaagtttgcagcttttatttgaaaatcgatactcaggatattgaagcatcccactacagaggctacggaaactctgattacaaacgcatagtagcatgtgccagctacaagcccagcagggaagaatcatcatctggatcctctcatgcggtgatggacatctccttgcctactggaatcagtgcaaatgaagaagacttaaaagcccttgtggaaggggtggatcaactattcactgattaccaaatcaaagatggacatgttattctgcaactgaattcgattccctccagtgatttcctttgtgtacgattccggatatttgaactctttgaagttgggtttctcagtcctgccactttcacagtgtacgaataccacagaccagataaacagtgtaccatgttttatagcacttccaatatcaaaattcagaaagtctgtgaaggagccgcgtgcaagtgtgtagaagctgattgtgggcaaatgcaggaagaattggatctgacaatctctgcagagacaagaaaacaaacagcatgtaaaccagagattgcatatgcttataaagttagcatcacatccatcactgtagaaaatgtttttgtcaagtacaaggcaacccttctggatatctacaaaactggggaagctgttgctgagaaagactctgagattaccttcattaaaaaggtaacctgtactaacgctgagctggtaaaaggaagacagtacttaattatgggtaaagaagccctccagataaaatacaatttcagtttcaggtacatctaccctttagattccttgacctggattgaatactggcctagagacacaacatgttcatcgtgtcaagcatttttagctaatttagatgaatttgccgaagatatctttttaaatggatgctaaaattcctgaagttcagctgcatacagtttgcacttatggactcctgttgttgaagttcgtttttttgttttcttctttttttaaacattcatagctggtcttatttgtaaagctcactttacttagaattagtggcacttgcttttattagagaatgatttcaaatgctgtaactttctgaaataacatggccttggagggcatgaagacagatactcctccaaggttattggacaccggaaacaataaattggaacacctcctcaaacctaccactcaggaatgtttgctggggccgaaagaacagtccattgaaagggagtattacaaaaacatggcctttgcttgaaagaaaataccaaggaacaggaaactgatcattaaagcctgagtttgctttcaaaaaaaaaaaaaaaaaa

SEQ ID NO: 2 - DNA, 5384 bp, Macaca mulatta
  variation 2876..2895: note = a, c, t, g, unknown or other
  source 1..5384: mol_type = genomic DNA
catgatttcctgctacctccaaccatgggccttttgggaatactttgttttttaatcttcctgggaaaaacttggggacaggagcaaacatatgtcatttcagcaccaaaaatattccgtgttggagcatctgaaaacattgtgattcaagtttatggatacactgaagcatttgatgcaacaatctctattaaaagttatcctgataaaaaatttagttactcctcaggccatgttcatttatcctcagagaataaattccaaaactcggcagtcttaacaatacaaccaaaacaattacctggaggacaaaaccaagtttcttatgtgtatttggaagttgtatcaaagcatttttcaaaatcaaaaaaaattccaataacctatgacaatggatttctcttcattcatacagacaaacctgtttatactccagaccaatcagtaaaggttagagtttattcgttgaatgatgacttgaagccagccaaaagagaaactgtcttaactttcatagatcctgaaggatcagaaattgacatggtagaagaaattgatcatattggaattatctcttttcctgacttcaagattccgtctaatcctagatatggtatgtggatgatccaggctaaatataaagaggacttttcaacaactggaactgcattttttgaagttaaagaatatgtcttgccacatttttctgtctcagtagaaccagaaagtaatttcattggttataagaactttaagaattttgaaattactataaaagcaagatatttttataataaagtagtcactgaggctgatgtttatatcacatttggaataagagaagacttaaaagatgatcaaaaagaaatgatgcaaacagcaatgcaaaacacaatgttgataaatggaattgctcaagtcacatttgattctgaaacagcagtcaaagaactgtcatactacagtttagaagatttaaacaacaagtacctttatattgctgtaacagtcatagagtctacaggtggattttctgaagaggcagaaatacctggcatcaaatatgtcctctctccctacaaactgaatttggttgctactcctcttttcctgaagcctgggattccatattccatcaaggtgcaggttaaagatgcgcttgaccagttggtaggaggggtcccagtaacactgaatgcacaaacaattgatgtcaaccaagagacatctgacttggagccaaggaaaagtgtaacacgtgttgatgatggagtagcttcgtttgtggttaatctcccatctggagtgacggtgctggagtttaatgtcaaaactgatgctccagatcttccagacgaaaatcaggccagggaaggttaccgagcaatagcatactcatctctcagccaaagttacctttatatcgattggactgataaccacaaggctttgctagtgggagaatatttgaatattattgttacccccaaaagcccatatattgacaaaataactcactataattacttgattttatccaagggcaaaattatccactttggcacaagggagaaactttcagatgcatcttatcaaagtataaacattccagtaacgcagaacatggttccttcatcccgactcctggtctattacatcgtcacaggagagcagacagcagaattagtgtctgattcagtctggttaaatattgaagaaaaatgtggcaaccagctccaggttcatctgtctcctgatgcagatacatattctccaggccaaactgtgtctcttaatatggtaactgggatggattcctgggtggcattaacagcagtggacagcgctgtgtatggagtccaaagaagagccaaaaagcccttggaaagagtatttcaattcttagagaagagtgatctgggctgtggggcaggtggtggcctcaacaatgccaatgtgttccacctagctggacttaccttcctcactaatgcaaatgcagatgactcccaagaaaatgatgaaccttgtaaagaaattatcaggccaagaagaatgctacaagagaagatagaagaaatagctgctaaatataaacatttagtagtgaagaaatgttgttacgatggagtccgtattaatcatgatgaaacctgtgagcagcgagctgcacggattagtgtagggccgagatgcgtcaaagctttcactgaatgttgtgtcgtcgcaagccagctccgtgctaataactctcataaagacttgcaattgggaaggctacacatgaagaccctgttaccagtaagcaagccagaaattcggagttattttccagaaagctggttatgggaagttcatcttgttcccagaagaaaacagttgcagtttgccctacctgattctgtaactacctgggaaattcaaggtgttggcatttcaaacagtggtatatgtgttgctgatactattaaggcaaaggtgttcaaagatgtcttcctggaaatgaatataccatattctgttgtacgaggagaacaggtccagttgaaaggaactgtttacaactataggacttctgggatgcagttctgtgttaaaatgtctgctgtggagggaatctgcacttcagaaagcccagtcattgatcatcagggcacaaagtcctccaaatgtgtgcgacagaaagtagagggctcctctaatcacttggtgacctttactgtgcttcctctggaaattggccttcagaacatcaatttctcactggagacttcgtttggaaaagaaatcttagtaaaatcgttacgagtggtgccagaaggtgtcaaaagggaaagctattctggtattactttggatcctaggggtatttatgnnnnnnnnnnnnnnnnnnnncgaaaggagttcccatacaggataccattagatttggtccccaaaacagaaatcaaaaggattttgagtgtaaaaggactgcttgtaggtgagatcttgtctgcagttctaagtcgggaaggcatcaatatcctaacccacctccccaaagggagtgcagaggcggagctgatgagcgttgtcccagtattctatgtttttcactacctggaaacaggaaatcattggaacatttttcattccgacccattaattgaaaagcggaacctggagaaaaaattaaaagaagggatggtgagcattatgtcctacagaaatgctgactattcttacagcgtgtggaagggtggcagtgctagcacttggttaacagcttttgctttaagagtacttggacaagtacataaatatgtagagcagaaccaaaattcaatatgtaattctttattgtggctggttgagaattatcagttagataatggatccttcaaggaaaattcacagtatcaaccaataaaattacagaaaatcaacacagctctaattaaagctgacacctttctgcttgaaaatacactgccagcccagagcacctttacattggccatttctgcctatgctctttccctgggagataaaactcacccacagttttgttcaattgtttcagctttgaagagagaagctttggttaaaggtaatccacccatttatcgtttttggaaagacagtcttcaacataaagacagctctgtacctaacactggtacagcacgtatggtagaaacaactgcctatgctttactcaccagtctgaacttgaaagacataaattatgttaacccaatcatcaaatggctatcagaagagcagaggtatggaggtggcttttattcaacccaggacacaatcaatgccatcgagggcctgacagaatattcactcctggttaaacagctccgcttgaatatggacatcgatgttgcttacaagcataaaggtcccttacataattataaaatgacagacaagaatttccttgggaggccagtagaggtgcttctcaatgatgacctcgttgtcagtacaggatttggcagtggcttggctacggtacatgtaacaactgtagttcacaaaaccagtacctctgaggaagtttgcagcttttatttgaaaattgatactcaggatattgaagcatcccactacagaggctacggaaactctgattacaaacgcatagtagcatgtgccagctacaagcccagcaaggaagaatcatcttctggatcctctcatgcagtgatggacatctccttgcctactggaatcaatgcaaatgaagaagacttaaaagctcttgtggaaggggtggatcagctattcactgattaccaaataaaagatggacatgttattctgcaactgaattcgatcccctccagtgatttcctttgtgtacgattccggatttttgaactctttgaagttgggtttcttagtcctgccactttcacagtgtatgaataccacagaccagataaacagtgtaccatgttttatagcacttccaatatcaaaattcagaaagtctgtgaaggagccacgtgcaagtgtatagaagctgattgtgggcaaatgcagaaagaattggatctgacaatctctgcagagactagaaaacaaacagcatgtaacccagagattgcatatgcttataaagttatcatcacatccatcactacagaaaatgtttttgtcaagtacaaggcaacccttctggatatctacaaaactggggaagctgttgctgaaaaagactctgaaatcaccttcattaaaaaggtaacctgcactaacgctgagctggtgaaaggaagacagtacttaattatggggaaagaagctctccagataaaatacaatttcactttcaggtacatctaccctttagattccttgacctggattgaatactggcctagagacacaacatgttcatcgtgtcaagcatttttagctaatttagatgaatttgctgaagacatctttttaaatggatgctaaaattcctgaagttcagctgcatacagtttgcacttatggactcctgttgttgaagtttgtttttttttctcgtttttttgtctttaaacattcacagctggtcttatttgtaaagctcactttacttagaattagtggcacttgcttttattagagaatgattttaaacgctgtaactttctgaaataacatggccttggagggcatgaagacagatactcctccaaggttattggacaccggaaacaataaattagaacacctcctcaaacctaccacttaggaatgtttgctggagccgaaagaacagtccattgaaatggagtattacaaaaacatggcctttgcttgaaagaaaataccaggggacaggaaactgatcattaaagcctgagtttgctttcaaactgtgctaaaaa

SEQ ID NO: 3 - DNA, 5448 bp, Mus musculus
  source 1..5448: mol_type = genomic DNA
tttaaaaggaaagtggttacagggaggccatgcccatgggtttatgccgctaccagccatgggtctttggggaatactttgtcttttaattttcctggacaaaacttggggacaggaacaaacctacgtcatttcagcacccaaaatcctccgggtcggctcgtctgaaaatgtggtaattcaagtccatggctacactgaagcatttgatgcaactctttctctaaaaagctatcctgacaaaaaagtcaccttctcttcaggctatgttaatttgtccccggaaaacaaattccaaaacgcggcactgttgacactacagcccaatcaagttcctagagaagaaagcccagtctctcacgtgtatctggaagttgtgtcaaaacacttttcaaaatcaaagaaaataccaattacctataacaatggaattctcttcatccatacagacaaacctgtttacacgccggaccagtcagtaaagatcagagtctattctctgggtgacgacttgaagccagccaaacgggagactgtcttaactttcatagaccccgaaggatcagaagttgacattgtagaagaaaatgattacaccggaattatctcttttcctgacttcaagattccatctaatcccaagtatggtgtttggacaattaaagctaactataagaaggattttacaacaactggaactgcatactttgaaattaaagaatatgtcttgccacgattctctgtttcaatagaactagaaagaaccttcattggctataaaaactttaagaactttgaaatcactgtgaaagcaagatatttttataataaagtggtacctgatgctgaagtgtatgccttttttggattgagagaggacataaaagatgaggagaagcagatgatgcacaaagccacacaagccgcaaagttggttgacggagttgctcagatctcttttgattctgaaacagcagttaaagagctgtcctacaacagtctagaagacttaaacaacaagtacctttatattgcagtaacagtcacagaatcttcaggtggattttcagaagaggcagaaatccctggagtcaaatatgtcctctctccctacacactgaatttggtcgctactcctcttttcgtgaagcccgggattccattttccatcaaggcacaggttaaagattcactcgagcaggcggtaggaggggtcccagtaactctgatggcacaaacagtcgatgtgaatcaagagacatctgacttggaaacaaagaggagcatcactcatgacactgatggagtagctgtgtttgtgctgaacctcccatcaaatgtgacggtgctaaagtttgagatcagaactgatgacccagaacttcccgaagaaaatcaagccagcaaagagtacgaagcagttgcgtactcgtctctcagccaaagttacatttacatcgcttggactgaaaactacaagcccatgcttgtgggagaatacctgaatattatggttacccccaagagcccatatatcgacaaaataactcactataattacttgattttatccaaaggcaaaattgtacagtacggcacaagagagaaacttttctcctcaacttatcaaaatataaatattccagtgacacagaacatggttccttcagcacgactcctggtctattacatagtcacaggggagcaaacagcagaattagtggctgacgcagtctggataaatattgaggagaagtgtggcaaccagctccaggtccatctgtctccagatgaatatgtgtattctccaggccaaactgtgtcccttgacatggtgactgaagcagactcatgggtagcactatcagcagtggacagagctgtgtataaagtccagggaaacgccaaaagggccatgcaaagagtctttcaagctttggatgaaaagagtgacctgggctgtggggcaggtggtggccatgacaatgcagatgtattccatctagctgggctcaccttcctcaccaacgcaaacgcagatgactcccattatcgtgatgactcttgtaaagaaattctcaggtcaaagagaaacctgcatctcctaaggcagaaaatagaagaacaagctgctaagtacaaacatagtgtgccaaagaaatgctgctatgacggagcccgagtgaacttctacgaaacctgtgaggagcgagtggcccgggttaccataggccctctctgcatcagggccttcaacgagtgctgtactattgcgaacaagatccgaaaagaaagcccccataaacctgtccaactgggaaggatccacattaagaccctgttaccagtgatgaaggcagatatccgaagctactttccagagagctggctatgggaaattcatcgcgttcccaaaagaaaacagctgcaggtcacgctgcctgactcactaacgacttgggaaattcaaggcattggcatttcagacaatggtatatgtgttgctgatacactcaaggcaaaggtgttcaaagaagtcttcctggagatgaacataccatattctgttgtgcgaggagaacagatccaattgaaaggaactgtttacaactatatgacctcagggacaaagttctgtgttaaaatgtctgctgtggaggggatctgcacttcaggaagctcagctgctagccttcacacctccaggccctccagatgtgtgttccagaggatagagggctcgtccagtcacttggtgaccttcaccctgcttcctctggaaattggccttcactccataaacttctcactagagacctcatttgggaaagacatcttagtaaagacattacgggtagtgccagaaggagtcaagagggaaagctatgccggcgtgattctggaccctaagggaattcgtggtattgttaacagacgaaaggaattcccatacaggatcccattagatttggtccccaagaccaaagttgaaaggattttgagtgtcaaaggactgcttgtaggggagttcttgtccacggttctgagtaaggaaggcatcaacatcctaacccacctccccaagggcagtgcagaggcagagctcatgagcatagctccggtgttctatgttttccactacctggaagcaggaaaccattggaatattttctatcctgatacactgagtaaaagacagagcctggagaaaaaaataaaacaaggggtggtgagcgtcatgtcctacagaaacgctgactattcctacagcatgtggaagggggcgagcgctagtacctggctgacagcttttgctctgagagtgcttggacaggtggccaagtatgtaaaacaggatgaaaactcaatttgtaactctttgctatggctggttgagaagtgtcagctggaaaacggctctttcaaggaaaattcccaatatctaccaataaaattacagggtactttgcctgctgaagcccaagagaaaactttgtatcttacagccttttctgtgattggaattagaaaggcagttgacatatgccccaccatgaaaatccacacagcgctagataaagccgactccttcctgcttgaaaacaccctgccatccaagagcaccttcacactggccattgtagcctatgctctttccctaggagacagaacccacccgaggtttcgtctaattgtgtcggccctgaggaaggaagcttttgttaaaggtgatccgcccatttaccgttactggagagataccctcaaacgtccagacagctctgtgcccagcagcggcacagcaggtatggttgaaaccacagcctatgctttgctcgccagcctgaaactgaaggatatgaattacgccaaccccatcatcaagtggctatctgaagagcagaggtatggaggcggcttttattccacccaggatacgattaatgccatcgagggcctgacagaatattcactcctgttaaaacaaattcatttggatatggacatcaatgtcgcctacaaacacgaaggtgacttccacaagtataaggtgacagagaagcatttcctggggaggccagtggaggtatctctcaatgatgaccttgttgtcagcacaggctacagcagtggcttggccacagtatatgtaaaaactgtggttcacaaaattagtgtctctgaggaattttgcagcttttacttgaaaattgatacccaagatattgaagcatccagccacttcaggctcagtgactctggattcaagcgcataatagcatgtgccagctacaagcccagcaaggaggagtcaacatccgggtcctcccatgcagtaatggatatatcactgccgactggaatcggagcaaacgaggaagatttacgggctcttgtggaaggagtggatcaactactaactgattaccagatcaaagatggccatgtcattctgcaactgaattcgatcccctccagagatttcctctgtgtccggttccggatatttgaacttttccaagttgggtttctgaatcctgctaccttcacggtgtacgagtatcacagaccagataagcagtgcaccatgatttatagcatttctgacaccaggcttcagaaagtctgtgaaggagcagcttgcacatgtgtggaagctgactgtgcgcaactgcaggcagaagtagacctagccatctctgcagactccagaaaagagaaagcctgtaaaccagagactgcatatgcttataaagtcaggatcacatcagccactgaagaaaatgtttttgtcaagtacactgcgactcttctggtcacttacaaaacaggggaagctgctgatgagaattcggaggtcaccttcattaaaaagatgagctgtaccaatgccaacctggtgaaagggaagcagtatttaatcatgggcaaagaggttctgcagatcaaacacaatttcagtttcaagtatatataccctctagattcctccacctggattgaatattggcccacagacacaacgtgtccatcctgtcaagcatttgtagagaatttgaataactttgctgaagacctctttttaaacagctgtgaatgaaaagttctgctgcacgaagattcctcctgcggcggggggattgctcctcctctggcttggaaacctagcctagaatcagatacactttctttagagtaaagcacaagctgatgagttacgactttgtgaaatggatagccttgaggggaggcgaaaacaggtcccccaaggctatcagatgtcagtgccaatagactgaaacaagtctgtaaagttagcagtcaggggtgttggttggggccggaagaagagacccactgaaactgtagccccttatcaaaacatatccttgcttgaaagaaaaataccaaggacagaaaatgccataaaatcttgactttgcactc

SEQ ID NO: 4 - DNA, 4497 bp, Rattus norvegicus
  source 1..4497: mol_type = genomic DNA
atggatagcacagagaccgacagatgtcctacagcccgccatcatctttccggaaacattaactcagtgcttgctgcccttgtaggtgggttttcggaagaggcagaaattcctggcatcaaatacgtcctctctccctatacactgaatttggtcgctacccctcttttcctgaagcctgggattccattttccatcaaggtacaggttaaggattcactcgagcagttggtaggaggggtcccagtaactctgatggcacaaacagtcaatgtgaatcaagagacatctgacttggaaccaaagaggagcatcacacactctgctgatggagtggcttcatttgtggtgaacctcccatcagaagtgacatcactgaagtttgaggtcaaaactgatgccccggaacttcccgaagaaaatcaagccagcaaagaatatgaagcagttacatactcatccctcagccagagttacatttacattggctggactgaaaactacaagcccatgcttgtgggagaatatctgaatattatcgtcacccccaagagtccatatattgacaaaataactcactataattacttgattttatccaaaggcaaaattgtacagtatggcacaaaggagaaacttctctattcatcttatcaaaatataaacatcccagtgacacaggacatggttccttcagcgcggctcctggtctattacatagtcacgggggagcagacagcagaattggtggctgacgcagtctggataaacattgaggagaagtgtggcaaccagctccaggtccatctgtctccagataaagacgtgtattctccaggccaaactgtgtcccttgacatggtgactgaagcagactcatgggtggcactatctgcggtggacagcgctgtgtatggagtccggggaaaagccaaaagggccatgcaaagagtgttccaagcttttgatgacaagagtgacctgggctgtggggcaggtggtggccgtgacaatgtagatgtattccatctagctgggctcaccttcctcaccaatgcaaacgcagatgactcccaataccacgatgactcttgtaaggaaattctcaggccaaagagagacctgcagctcctgcatcagaaagtggaagaacaagctgctaaatacaaacaccgtgtgcccaagaaatgctgttatgatggagcccgagaaaacaaatacgaaacctgtgagcagcgagttgcccgggtgaccataggcccacactgcatcagggccttcaacgagtgttgtactattgcggataagatccgaaaagaaagccaccacaaaggcatgctgttgggaaggatccaaataaaggccctgttaccagtgatgaaggcagaaatccgaagctactttccagagagctggctatgggaagttcatcgtgttcccaaaagaaaccagctgcaggttgcactgcctgactcactgacgacctgggaaattcaaggcatcggcatctcagacaatggtatatgtgttgctgacacactcaaggcaaaggtgttcaaagatgtcttcctggagatgaacataccatattctgttgtacgaggggagcagatccaattgaagggaaccgtttacaattataggacctctgggacaatgttctgtgttaaaatgtctgccgtggagggaatctgcactccaggaagctcggctgctagccctcagacctctaggtcctccagatgtgtgcgccagagaatagagggctcctccagtcacttggtgaccttcagcctgcttcctctggaaattggccttcactccataaacttctcactagagacttcatttgggaaagaaatcttagtgaagacattacgggtagtgccagaagggatcaaaagggaaagctatgctggtgtgactctggaccccaggggagtttatggtattgttaacagacgaaaggaattcccatacaggataccattagatttggtccccaaaaccaacgtcaaaaggattttgagtgtaaaaggactgcttataggggaattcttgtccacggttctgagtaaagaaggcatcgacatcctaacccacctccccaagggcagcgccgaggcagaactcatgagcatagtcccggtgttctacgttttccactacctggaagcaggaaaccattggaatattttccaccctgatacgttagctagaaaacagagcctgcagaaaaaaataaaagaagggctggtgagcgtcatgtcctacagaaacgctgactattcctacagcatgtggaagggagcaagctctagtgcctggctgacagcttttgctctgagagtgcttggacaggtgaacaagtatgtgaaacaagaccaatactcgatctgtaactccttgttatggctgattgagaagtgtcagctggaaaacggatctttcaaggaaaattcccaatatctaccaataaaattacagggtactttgcctgctgaagcccaagagaacactttatatcttacagccttttctgtgattggaattagaaaggctattggcatatgccccacggagaaaatctacacagcgctggctaaagctgactccttcctacttgaaaggaccctgccttccaagagcaccttcaccctggccattgtggcctatgctctctccctgggagacagaacccacccgaagtttcgttctattgtgtcagccctgaagagggaagctttggttaaaggagacccgcccatttaccgtttctggagagacactctccaacgtccagacagctcagcacccaacagcggcacagcaggtatggtagaaaccacggcctatgctttgctcaccagcctgaacctgaaggagacgagttatgtcaacccgatcatcaagtggctatctgaggagcagaggtatggaggcggcttttattccacccaggataccattaacgccatcgagggcctgacagagtattcactcctggttaaacaacttcatttggatatggatatcaatgtctcctacaaacacaaaggggatttctaccagtataaagtgacagagaagaacttcctcgggaggccagtggaggtacccctcaatgatgacctcatcgtcaccacaggctatagcagtggcttggctacagtatatgtaaaaactgtggttcacaaaactagtgtcgctgaggaattttgcagcttttacttgaaaattgatacccaagaagttgaagcctccagctacctcagctacagtgactcgggacacaagcgcataatagcctgtgccagctacaagcccagcaaggaggagtcagcatctgggtcctcccatgcagtaatggatatactgctgccgaccggaatcggagcaaaccaagaagatttacgagctcttgtggaaggagtagatcaactcctaactgattaccagatcaaagacagtcatgttattctgcaattgaattcgattccctccagagatttcctttgtgttcggttccggatatttgaacttttccaagttgggtttctgaatcctgctacgttcacggtgtacgagtatcacagaccagataagcagtgtaccatgatttacagcacttctgacaccaaccttcagagagtctgtgaaggagcggcatgcaaatgcgttgaagctgattgtgggcaactgcaggcagaactggacctggccatctctgcagacaccaggaaagaaacagcatgtaaaccagagattgcatatgcttataaggtcaggatcacgtcggccacggaagaaaacatttttgtcaagtacactgcgacgcttctggatatttacaaaacaggggaagccgctgctgagaaggactctgagatcaccttcattaaaaagataagctgtaccaacgccaacctggtgaaaggaaagcaatatttaatcatgggcaaagaggctctgcagatcaaacacaatttcagtttcaagtatatataccctctagattcctccacctggattgaatattggcccacagacacaacgtgtccatcctgccaagcgtttgtagctaatttggacgagttcgctgaagacatctttctaaatggctgtgaaaatgcctgaggaagttctgctgcgtggccttcccgggtactcctgttggtggctcctaggagccaggatcgcttggaaacttagcctagaatcggatacattttctttatagtaaagcgtaagttgaagagttactttgtgaaacaaaatagccttgtggagagccgaaggcaggtcccccaaggctattggacatcagcaccaataagctggaacaagtctgtaacgttagcagccaggggtgtttgttggggccggaagaagagactcactgaaattgtagccccttaggaaaacatggtcttgcttgaaaaaaaaaataccaaggacagaaaatgccataaaagcttgactttgcactcaactgta

SEQ ID NO: 5 - DNA, 5480 bp, Homo sapiens
  source 1..5480: mol_type = genomic DNA
ttttttttttttttttttgaaagcaaactcaggctttaatgatcagtttcctgttccttggtattttctttcaagcaaaggccatgtttttgtaatactccctttcaatggactgttctttcggccccagcaaacattcctgagtggtaggtttgaggaggtgttccaatttattgtttccggtgtccaataaccttggaggagtatctgtcttcatgccctccaaggccatgttatttcagaaagttacagcatttgaaatcattctctaataaaagcaagtgccactaattctaagtaaagtgagctttacaaataagaccagctatgaatgtttaaaaaaagaagaaaacaaaaaaacgaacttcaacaacaggagtccataagtgcaaactgtatgcagctgaacttcaggaattttagcatccatttaaaaagatatcttcggcaaattcatctaaattagctaaaaatgcttgacacgatgaacatgttgtgtctctaggccagtattcaatccaggtcaaggaatctaaagggtagatgtacctgaaactgaaattgtattttatctggagggcttctttacccataattaagtactgtcttccttttaccagctcagcgttagtacaggttacctttttaatgaaggtaatctcagagtctttctcagcaacagcttccccagttttgtagatatccagaagggttgccttgtacttgacaaaaacattttctacagtgatggatgtgatgctaactttataagcatatgcaatctctggtttacatgctgtttgttttcttgtctctgcagagattgtcagatccaattcttcctgcatttgcccacaatcagcttctacacacttgcacgcggctccttcacagactttctgaattttgatattggaagtgctataaaacatggtacactgtttatctggtctgtggtattcgtacactgtgaaagtggcaggactgagaaacccaacttcaaagagttcaaatatccggaatcgtacacaaaggaaatcactggagggaatcgaattcagttgcagaataacatgtccatctttgatttggtaatcagtgaatagttgatccaccccttccacaagggcttttaagtcttcttcatttgcactgattccagtaggcaaggagatgtccatcaccgcatgagaggatccagatgatgattcttccctgctgggcttgtagctggcacatgctactatgcgtttgtaatcagagtttccgtagcctctgtagtgggatgcttcaatatcctgagtatcgattttcaaataaaagctgcaaacttcctcagaggtactggttttgtgaactacagttgttacatgtactgtagccaagccactgccaaatcctgtactgacaatgaggtcatcattgagaagcacctctactggcctcccaaggaaattcttgtctgtcattttataattatgtaaggcacctttatgcttgtaagaaacatcgatgtccatactcaagcggagttgtttaaccaggagtgaatattccgtcaggccctcaatggcattgattgtgtcctgggttgaataaaagccacctccatacctctgctcttctgatagccatttgatgactgggttaacataatttatatctttcaagttcagactggtgagtaaagcataggcagttgtttctaccatacgtgccgtaccagtgttaggtacagagctgtctttatgctgaagattgtctttccaaaaacgataaatgggtggattacctttaaccaaagcttctctcttcaaagctgaaacaattgaacgaaactgtgggtgagttttatctcccagggaaagagcatacgcagaaatggccaatgtaaaggtgctctgggctggcagtgtattttcaagcagaaagttgtcagctttaattagagctgtgtcgattttcaccagggggcatatatcgaaagcctttctaattccaatcacagtaaaggctgtaagatataagctgttctctcgggcttcaacaggcaaggtaccctgtaattttattggttgatactgtgaattttccttgaaagatccattatctaattgataattctcaactagccacaataaagaattacaaattgaattttggttctgctctacgtatttatttacttgtccaagtactcttaaagcaaaagctgttaaccaagtgctagcacttccacccttccacacactgtaagagtagtcagcatttctgtaggacataatgctcaacatcccttcttttaattttttcttcagtttctgcttttcaattaatgggtcagaatgaaaaatgttccaatgatttcctgtttccaggtagtgaaaaacatagaatactgggacaacgctcatcagctccgcctctgcactccctttggggaggtgggttaggatattgatgccttcctgacttagaactgcagacaagatctcacctacaagcagtccttttacactcaaaatccttttgatttctgttttggggaccaaatctaagggtatcctgtatgggaactcctttcgtctgctaatggtaccataaatacccctaggatccaaagtaacaccagaatagctttcccttttgacaccttctggcaccactcgtaatgtttttactaagatttcttttccaaaccaagtctccagtgaaaaattgatgttgtgaaggccaatttccagaggaagcacagtgaatgtcaccaagtgactggaggagccctctactttctggcgcacacatttggaggactttgtgccctgatgatcaatgactgggctttccgaagtgcagattccctccacagcagacattttaacacagaactgcatcccagaagtcctatagttgtaaacagttcctttcaattggatctgttctcctcgtacaacagaatatggtatattcatttccaggaagacatctttgaacacctttgccttgacagtatcagcaacacatataccagtgtttgaaatgccaacgccttgaatttcccaggtggttagagaatcaggtagggcaaactgcaactgttttcttctgggaacaagatgaacttcccacaaccagctttctggaaaataactccgaatttctggcttgcttactggtaacagggtcttcatgtgtagccttcccaattgcatgtctttatgagagatattagcacggagctggcttgcgacgacacaacattcagtgaaagctttgatgcatcttggccctaaactaatccgtgcagctcgctgctcacaggtttcatcattattaacgcaggctccatcgtaacaacatttcttcactactgaatgtttatatttagcagctatttcttctatcttcttttgcagcgttcttcttggcctgagaatttctttacaaggttcatcattttcttgggagtcatctgcatttgcattagtgaggaaggtaagtccagctaggtggaacacattggcattgttgaggccaccacctgccccacagcccagatcactcttctctaagaattgaaatactctttccaagggctttttggctcctctttggactccatacacagcactgtccactgctgctaatgccacccaggaatccattccagttgccatattaagagacacagtttggcctggagaatatgcatctgcatcaggagacagatgaacctggagctggttgccacatttttcttcaatatttaaccagactgaatcagacactaattctgctgtctgttctcctgtgacgatgtaatagaccagaagtcgggatgaaggaaccatgttctgtgttactggaatgtttatactttgataagatgcatctgaaaatttctccctcgtgccaaagtggataattttgcccttggataaaatcaagtaattatagtgagttattttgtcaatatatgggcttttgggggtaacaataatattcagatgttctcccactagcaaagccttatggttatcagtccaatcaatataaaggtaactttggctgagagatgagtatgctattgctcggtaaccttccctggcctgattttcttctggaagatctggagcatcagttttgacattaaactccagcaccgtcactccagatgggagattaagcacaaaggaagctactccatcatcaacacgtgttacacttttgcttggatccaagtcagatgtctcttggtttacatcaattgtttgtgcattcagtgttactgggactcctcctaccaactggtcaagcgaatctttaacctgcaccttgatgggatatggaatcccaggcttcaggaaaagaggagtagcaaccaaattcagtttgtagggagagaggacatatttgatgccaggtatttctgcctcttcagaaaatccacctgtagactctatgactgttacagcaatataaaggtacttgttgtttaaatcttctaaactgtagtatgacagttctttgactgctgtttcagaatcaaatgtgacttgagcaattccatttatcaacattgtgttttgcattgctgtttgcatcatttctttttgatcatcttttaagtcttctcttattccaaatgtgatataaacgtcagcctcagtgactactttattataaaaatatcttgcttttatagtaatttcaaaattcttaaagttcttgtaaccaatgaaattatattctggctcgattgagacagaaaaatgtggcaagacatattctttaacttcaaaatatgcggttccagttgttgaaaagtcctctttatatttagccttgatcgtccacataccatatctaggattagacggaatcttgaagtcaggaaaagagataattccaatatgatcaatttcttctaccatgtcaacttctgatccttcaggatctatgaaagttaagacagtttctcttttggctggcttcaagtcgtcattcaacgaataaactctaacttttactgactggtctggagtataaacaggtttgtctgtatgaatgaagagaaatccattgtcataggttattggcattctttttgattttgaaaaatgctttgatacaacttccaaatacacataagaaactgggttttgtcctccaggcaattgttttggttgtattgttaagattgcagagttttggaatttattctctgaggataaatgaacatggcctgaggagtaactaaattttttatcaggataacttttaatagagattgttgcatcaaatgcttcagtgtatccataaacttgaatcacaatattttcagatgctccaacacggaatatttttggtgctgaaatgacatatgtttgctcctgtccccaggttttccccaggaagattaaaaaacaaagtattcccaaaaggcccatggttggaggtagcaggaaaccacggatata

SEQ ID NO: 6 - DNA, 5384 bp, Macaca mulatta
  variation 2490..2509: note = a, c, t, g, unknown or other
  source 1..5384: mol_type = genomic DNA
tttttagcacagtttgaaagcaaactcaggctttaatgatcagtttcctgtcccctggtattttctttcaagcaaaggccatgtttttgtaatactccatttcaatggactgttctttcggctccagcaaacattcctaagtggtaggtttgaggaggtgttctaatttattgtttccggtgtccaataaccttggaggagtatctgtcttcatgccctccaaggccatgttatttcagaaagttacagcgtttaaaatcattctctaataaaagcaagtgccactaattctaagtaaagtgagctttacaaataagaccagctgtgaatgtttaaagacaaaaaaacgagaaaaaaaaacaaacttcaacaacaggagtccataagtgcaaactgtatgcagctgaacttcaggaattttagcatccatttaaaaagatgtcttcagcaaattcatctaaattagctaaaaatgcttgacacgatgaacatgttgtgtctctaggccagtattcaatccaggtcaaggaatctaaagggtagatgtacctgaaagtgaaattgtattttatctggagagcttctttccccataattaagtactgtcttcctttcaccagctcagcgttagtgcaggttacctttttaatgaaggtgatttcagagtctttttcagcaacagcttccccagttttgtagatatccagaagggttgccttgtacttgacaaaaacattttctgtagtgatggatgtgatgataactttataagcatatgcaatctctgggttacatgctgtttgttttctagtctctgcagagattgtcagatccaattctttctgcatttgcccacaatcagcttctatacacttgcacgtggctccttcacagactttctgaattttgatattggaagtgctataaaacatggtacactgtttatctggtctgtggtattcatacactgtgaaagtggcaggactaagaaacccaacttcaaagagttcaaaaatccggaatcgtacacaaaggaaatcactggaggggatcgaattcagttgcagaataacatgtccatcttttatttggtaatcagtgaatagctgatccaccccttccacaagagcttttaagtcttcttcatttgcattgattccagtaggcaaggagatgtccatcactgcatgagaggatccagaagatgattcttccttgctgggcttgtagctggcacatgctactatgcgtttgtaatcagagtttccgtagcctctgtagtgggatgcttcaatatcctgagtatcaattttcaaataaaagctgcaaacttcctcagaggtactggttttgtgaactacagttgttacatgtaccgtagccaagccactgccaaatcctgtactgacaacgaggtcatcattgagaagcacctctactggcctcccaaggaaattcttgtctgtcattttataattatgtaagggacctttatgcttgtaagcaacatcgatgtccatattcaagcggagctgtttaaccaggagtgaatattctgtcaggccctcgatggcattgattgtgtcctgggttgaataaaagccacctccatacctctgctcttctgatagccatttgatgattgggttaacataatttatgtctttcaagttcagactggtgagtaaagcataggcagttgtttctaccatacgtgctgtaccagtgttaggtacagagctgtctttatgttgaagactgtctttccaaaaacgataaatgggtggattacctttaaccaaagcttctctcttcaaagctgaaacaattgaacaaaactgtgggtgagttttatctcccagggaaagagcataggcagaaatggccaatgtaaaggtgctctgggctggcagtgtattttcaagcagaaaggtgtcagctttaattagagctgtgttgattttctgtaattttattggttgatactgtgaattttccttgaaggatccattatctaactgataattctcaaccagccacaataaagaattacatattgaattttggttctgctctacatatttatgtacttgtccaagtactcttaaagcaaaagctgttaaccaagtgctagcactgccacccttccacacgctgtaagaatagtcagcatttctgtaggacataatgctcaccatcccttcttttaattttttctccaggttccgcttttcaattaatgggtcggaatgaaaaatgttccaatgatttcctgtttccaggtagtgaaaaacatagaatactgggacaacgctcatcagctccgcctctgcactccctttggggaggtgggttaggatattgatgccttcccgacttagaactgcagacaagatctcacctacaagcagtccttttacactcaaaatccttttgatttctgttttggggaccaaatctaatggtatcctgtatgggaactcctttcgnnnnnnnnnnnnnnnnnnnncataaatacccctaggatccaaagtaataccagaatagctttcccttttgacaccttctggcaccactcgtaacgattttactaagatttcttttccaaacgaagtctccagtgagaaattgatgttctgaaggccaatttccagaggaagcacagtaaaggtcaccaagtgattagaggagccctctactttctgtcgcacacatttggaggactttgtgccctgatgatcaatgactgggctttctgaagtgcagattccctccacagcagacattttaacacagaactgcatcccagaagtcctatagttgtaaacagttcctttcaactggacctgttctcctcgtacaacagaatatggtatattcatttccaggaagacatctttgaacacctttgccttaatagtatcagcaacacatataccactgtttgaaatgccaacaccttgaatttcccaggtagttacagaatcaggtagggcaaactgcaactgttttcttctgggaacaagatgaacttcccataaccagctttctggaaaataactccgaatttctggcttgcttactggtaacagggtcttcatgtgtagccttcccaattgcaagtctttatgagagttattagcacggagctggcttgcgacgacacaacattcagtgaaagctttgacgcatctcggccctacactaatccgtgcagctcgctgctcacaggtttcatcatgattaatacggactccatcgtaacaacatttcttcactactaaatgtttatatttagcagctatttcttctatcttctcttgtagcattcttcttggcctgataatttctttacaaggttcatcattttcttgggagtcatctgcatttgcattagtgaggaaggtaagtccagctaggtggaacacattggcattgttgaggccaccacctgccccacagcccagatcactcttctctaagaattgaaatactctttccaagggctttttggctcttctttggactccatacacagcgctgtccactgctgttaatgccacccaggaatccatcccagttaccatattaagagacacagtttggcctggagaatatgtatctgcatcaggagacagatgaacctggagctggttgccacatttttcttcaatatttaaccagactgaatcagacactaattctgctgtctgctctcctgtgacgatgtaatagaccaggagtcgggatgaaggaaccatgttctgcgttactggaatgtttatactttgataagatgcatctgaaagtttctcccttgtgccaaagtggataattttgcccttggataaaatcaagtaattatagtgagttattttgtcaatatatgggcttttgggggtaacaataatattcaaatattctcccactagcaaagccttgtggttatcagtccaatcgatataaaggtaactttggctgagagatgagtatgctattgctcggtaaccttccctggcctgattttcgtctggaagatctggagcatcagttttgacattaaactccagcaccgtcactccagatgggagattaaccacaaacgaagctactccatcatcaacacgtgttacacttttccttggctccaagtcagatgtctcttggttgacatcaattgtttgtgcattcagtgttactgggacccctcctaccaactggtcaagcgcatctttaacctgcaccttgatggaatatggaatcccaggcttcaggaaaagaggagtagcaaccaaattcagtttgtagggagagaggacatatttgatgccaggtatttctgcctcttcagaaaatccacctgtagactctatgactgttacagcaatataaaggtacttgttgtttaaatcttctaaactgtagtatgacagttctttgactgctgtttcagaatcaaatgtgacttgagcaattccatttatcaacattgtgttttgcattgctgtttgcatcatttctttttgatcatcttttaagtcttctcttattccaaatgtgatataaacatcagcctcagtgactactttattataaaaatatcttgcttttatagtaatttcaaaattcttaaagttcttataaccaatgaaattactttctggttctactgagacagaaaaatgtggcaagacatattctttaacttcaaaaaatgcagttccagttgttgaaaagtcctctttatatttagcctggatcatccacataccatatctaggattagacggaatcttgaagtcaggaaaagagataattccaatatgatcaatttcttctaccatgtcaatttctgatccttcaggatctatgaaagttaagacagtttctcttttggctggcttcaagtcatcattcaacgaataaactctaacctttactgattggtctggagtataaacaggtttgtctgtatgaatgaagagaaatccattgtcataggttattggaattttttttgattttgaaaaatgctttgatacaacttccaaatacacataagaaacttggttttgtcctccaggtaattgttttggttgtattgttaagactgccgagttttggaatttattctctgaggataaatgaacatggcctgaggagtaactaaattttttatcaggataacttttaatagagattgttgcatcaaatgcttcagtgtatccataaacttgaatcacaatgttttcagatgctccaacacggaatatttttggtgctgaaatgacatatgtttgctcctgtccccaagtttttcccaggaagattaaaaaacaaagtattcccaaaaggcccatggttggaggtagcaggaaatcatg

SEQ ID NO: 7 - DNA, 5448 bp, Mus musculus
  source 1..5448: mol_type = genomic DNA
gagtgcaaagtcaagattttatggcattttctgtccttggtatttttctttcaagcaaggatatgttttgataaggggctacagtttcagtgggtctcttcttccggccccaaccaacacccctgactgctaactttacagacttgtttcagtctattggcactgacatctgatagccttgggggacctgttttcgcctcccctcaaggctatccatttcacaaagtcgtaactcatcagcttgtgctttactctaaagaaagtgtatctgattctaggctaggtttccaagccagaggaggagcaatccccccgccgcaggaggaatcttcgtgcagcagaacttttcattcacagctgtttaaaaagaggtcttcagcaaagttattcaaattctctacaaatgcttgacaggatggacacgttgtgtctgtgggccaatattcaatccaggtggaggaatctagagggtatatatacttgaaactgaaattgtgtttgatctgcagaacctctttgcccatgattaaatactgcttccctttcaccaggttggcattggtacagctcatctttttaatgaaggtgacctccgaattctcatcagcagcttcccctgttttgtaagtgaccagaagagtcgcagtgtacttgacaaaaacattttcttcagtggctgatgtgatcctgactttataagcatatgcagtctctggtttacaggctttctcttttctggagtctgcagagatggctaggtctacttctgcctgcagttgcgcacagtcagcttccacacatgtgcaagctgctccttcacagactttctgaagcctggtgtcagaaatgctataaatcatggtgcactgcttatctggtctgtgatactcgtacaccgtgaaggtagcaggattcagaaacccaacttggaaaagttcaaatatccggaaccggacacagaggaaatctctggaggggatcgaattcagttgcagaatgacatggccatctttgatctggtaatcagttagtagttgatccactccttccacaagagcccgtaaatcttcctcgtttgctccgattccagtcggcagtgatatatccattactgcatgggaggacccggatgttgactcctccttgctgggcttgtagctggcacatgctattatgcgcttgaatccagagtcactgagcctgaagtggctggatgcttcaatatcttgggtatcaattttcaagtaaaagctgcaaaattcctcagagacactaattttgtgaaccacagtttttacatatactgtggccaagccactgctgtagcctgtgctgacaacaaggtcatcattgagagatacctccactggcctccccaggaaatgcttctctgtcaccttatacttgtggaagtcaccttcgtgtttgtaggcgacattgatgtccatatccaaatgaatttgttttaacaggagtgaatattctgtcaggccctcgatggcattaatcgtatcctgggtggaataaaagccgcctccatacctctgctcttcagatagccacttgatgatggggttggcgtaattcatatccttcagtttcaggctggcgagcaaagcataggctgtggtttcaaccatacctgctgtgccgctgctgggcacagagctgtctggacgtttgagggtatctctccagtaacggtaaatgggcggatcacctttaacaaaagcttccttcctcagggccgacacaattagacgaaacctcgggtgggttctgtctcctagggaaagagcataggctacaatggccagtgtgaaggtgctcttggatggcagggtgttttcaagcaggaaggagtcggctttatctagcgctgtgtggattttcatggtggggcatatgtcaactgcctttctaattccaatcacagaaaaggctgtaagatacaaagttttctcttgggcttcagcaggcaaagtaccctgtaattttattggtagatattgggaattttccttgaaagagccgttttccagctgacacttctcaaccagccatagcaaagagttacaaattgagttttcatcctgttttacatacttggccacctgtccaagcactctcagagcaaaagctgtcagccaggtactagcgctcgcccccttccacatgctgtaggaatagtcagcgtttctgtaggacatgacgctcaccaccccttgttttatttttttctccaggctctgtcttttactcagtgtatcaggatagaaaatattccaatggtttcctgcttccaggtagtggaaaacatagaacaccggagctatgctcatgagctctgcctctgcactgcccttggggaggtgggttaggatgttgatgccttccttactcagaaccgtggacaagaactcccctacaagcagtcctttgacactcaaaatcctttcaactttggtcttggggaccaaatctaatgggatcctgtatgggaattcctttcgtctgttaacaataccacgaattcccttagggtccagaatcacgccggcatagctttccctcttgactccttctggcactacccgtaatgtctttactaagatgtctttcccaaatgaggtctctagtgagaagtttatggagtgaaggccaatttccagaggaagcagggtgaaggtcaccaagtgactggacgagccctctatcctctggaacacacatctggagggcctggaggtgtgaaggctagcagctgagcttcctgaagtgcagatcccctccacagcagacattttaacacagaactttgtccctgaggtcatatagttgtaaacagttcctttcaattggatctgttctcctcgcacaacagaatatggtatgttcatctccaggaagacttctttgaacacctttgccttgagtgtatcagcaacacatataccattgtctgaaatgccaatgccttgaatttcccaagtcgttagtgagtcaggcagcgtgacctgcagctgttttcttttgggaacgcgatgaatttcccatagccagctctctggaaagtagcttcggatatctgccttcatcactggtaacagggtcttaatgtggatccttcccagttggacaggtttatgggggctttcttttcggatcttgttcgcaatagtacagcactcgttgaaggccctgatgcagagagggcctatggtaacccgggccactcgctcctcacaggtttcgtagaagttcactcgggctccgtcatagcagcatttctttggcacactatgtttgtacttagcagcttgttcttctattttctgccttaggagatgcaggtttctctttgacctgagaatttctttacaagagtcatcacgataatgggagtcatctgcgtttgcgttggtgaggaaggtgagcccagctagatggaatacatctgcattgtcatggccaccacctgccccacagcccaggtcactcttttcatccaaagcttgaaagactctttgcatggcccttttggcgtttccctggactttatacacagctctgtccactgctgatagtgctacccatgagtctgcttcagtcaccatgtcaagggacacagtttggcctggagaatacacatattcatctggagacagatggacctggagctggttgccacacttctcctcaatatttatccagactgcgtcagccactaattctgctgtttgctcccctgtgactatgtaatagaccaggagtcgtgctgaaggaaccatgttctgtgtcactggaatatttatattttgataagttgaggagaaaagtttctctcttgtgccgtactgtacaattttgcctttggataaaatcaagtaattatagtgagttattttgtcgatatatgggctcttgggggtaaccataatattcaggtattctcccacaagcatgggcttgtagttttcagtccaagcgatgtaaatgtaactttggctgagagacgagtacgcaactgcttcgtactctttgctggcttgattttcttcgggaagttctgggtcatcagttctgatctcaaactttagcaccgtcacatttgatgggaggttcagcacaaacacagctactccatcagtgtcatgagtgatgctcctctttgtttccaagtcagatgtctcttgattcacatcgactgtttgtgccatcagagttactgggacccctcctaccgcctgctcgagtgaatctttaacctgtgccttgatggaaaatggaatcccgggcttcacgaaaagaggagtagcgaccaaattcagtgtgtagggagagaggacatatttgactccagggatttctgcctcttctgaaaatccacctgaagattctgtgactgttactgcaatataaaggtacttgttgtttaagtcttctagactgttgtaggacagctctttaactgctgtttcagaatcaaaagagatctgagcaactccgtcaaccaactttgcggcttgtgtggctttgtgcatcatctgcttctcctcatcttttatgtcctctctcaatccaaaaaaggcatacacttcagcatcaggtaccactttattataaaaatatcttgctttcacagtgatttcaaagttcttaaagtttttatagccaatgaaggttctttctagttctattgaaacagagaatcgtggcaagacatattctttaatttcaaagtatgcagttccagttgttgtaaaatccttcttatagttagctttaattgtccaaacaccatacttgggattagatggaatcttgaagtcaggaaaagagataattccggtgtaatcattttcttctacaatgtcaacttctgatccttcggggtctatgaaagttaagacagtctcccgtttggctggcttcaagtcgtcacccagagaatagactctgatctttactgactggtccggcgtgtaaacaggtttgtctgtatggatgaagagaattccattgttataggtaattggtattttctttgattttgaaaagtgttttgacacaacttccagatacacgtgagagactgggctttcttctctaggaacttgattgggctgtagtgtcaacagtgccgcgttttggaatttgttttccggggacaaattaacatagcctgaagagaaggtgacttttttgtcaggatagctttttagagaaagagttgcatcaaatgcttcagtgtagccatggacttgaattaccacattttcagacgagccgacccggaggattttgggtgctgaaatgacgtaggtttgttcctgtccccaagttttgtccaggaaaattaaaagacaaagtattccccaaagacccatggctggtagcggcataaacccatgggcatggcctccctgtaaccactttccttttaaa

SEQ ID NO: 8 - DNA, 4497 bp, Rattus norvegicus
  source 1..4497: mol_type = genomic DNA
tacagttgagtgcaaagtcaagcttttatggcattttctgtccttggtattttttttttcaagcaagaccatgttttcctaaggggctacaatttcagtgagtctcttcttccggccccaacaaacacccctggctgctaacgttacagacttgttccagcttattggtgctgatgtccaatagccttgggggacctgccttcggctctccacaaggctattttgtttcacaaagtaactcttcaacttacgctttactataaagaaaatgtatccgattctaggctaagtttccaagcgatcctggctcctaggagccaccaacaggagtacccgggaaggccacgcagcagaacttcctcaggcattttcacagccatttagaaagatgtcttcagcgaactcgtccaaattagctacaaacgcttggcaggatggacacgttgtgtctgtgggccaatattcaatccaggtggaggaatctagagggtatatatacttgaaactgaaattgtgtttgatctgcagagcctctttgcccatgattaaatattgctttcctttcaccaggttggcgttggtacagcttatctttttaatgaaggtgatctcagagtccttctcagcagcggcttcccctgttttgtaaatatccagaagcgtcgcagtgtacttgacaaaaatgttttcttccgtggccgacgtgatcctgaccttataagcatatgcaatctctggtttacatgctgtttctttcctggtgtctgcagagatggccaggtccagttctgcctgcagttgcccacaatcagcttcaacgcatttgcatgccgctccttcacagactctctgaaggttggtgtcagaagtgctgtaaatcatggtacactgcttatctggtctgtgatactcgtacaccgtgaacgtagcaggattcagaaacccaacttggaaaagttcaaatatccggaaccgaacacaaaggaaatctctggagggaatcgaattcaattgcagaataacatgactgtctttgatctggtaatcagttaggagttgatctactccttccacaagagctcgtaaatcttcttggtttgctccgattccggtcggcagcagtatatccattactgcatgggaggacccagatgctgactcctccttgctgggcttgtagctggcacaggctattatgcgcttgtgtcccgagtcactgtagctgaggtagctggaggcttcaacttcttgggtatcaattttcaagtaaaagctgcaaaattcctcagcgacactagttttgtgaaccacagtttttacatatactgtagccaagccactgctatagcctgtggtgacgatgaggtcatcattgaggggtacctccactggcctcccgaggaagttcttctctgtcactttatactggtagaaatcccctttgtgtttgtaggagacattgatatccatatccaaatgaagttgtttaaccaggagtgaatactctgtcaggccctcgatggcgttaatggtatcctgggtggaataaaagccgcctccatacctctgctcctcagatagccacttgatgatcgggttgacataactcgtctccttcaggttcaggctggtgagcaaagcataggccgtggtttctaccatacctgctgtgccgctgttgggtgctgagctgtctggacgttggagagtgtctctccagaaacggtaaatgggcgggtctcctttaaccaaagcttccctcttcagggctgacacaatagaacgaaacttcgggtgggttctgtctcccagggagagagcataggccacaatggccagggtgaaggtgctcttggaaggcagggtcctttcaagtaggaaggagtcagctttagccagcgctgtgtagattttctccgtggggcatatgccaatagcctttctaattccaatcacagaaaaggctgtaagatataaagtgttctcttgggcttcagcaggcaaagtaccctgtaattttattggtagatattgggaattttccttgaaagatccgttttccagctgacacttctcaatcagccataacaaggagttacagatcgagtattggtcttgtttcacatacttgttcacctgtccaagcactctcagagcaaaagctgtcagccaggcactagagcttgctcccttccacatgctgtaggaatagtcagcgtttctgtaggacatgacgctcaccagcccttcttttatttttttctgcaggctctgttttctagctaacgtatcagggtggaaaatattccaatggtttcctgcttccaggtagtggaaaacgtagaacaccgggactatgctcatgagttctgcctcggcgctgcccttggggaggtgggttaggatgtcgatgccttctttactcagaaccgtggacaagaattcccctataagcagtccttttacactcaaaatccttttgacgttggttttggggaccaaatctaatggtatcctgtatgggaattcctttcgtctgttaacaataccataaactcccctggggtccagagtcacaccagcatagctttcccttttgatcccttctggcactacccgtaatgtcttcactaagatttctttcccaaatgaagtctctagtgagaagtttatggagtgaaggccaatttccagaggaagcaggctgaaggtcaccaagtgactggaggagccctctattctctggcgcacacatctggaggacctagaggtctgagggctagcagccgagcttcctggagtgcagattccctccacggcagacattttaacacagaacattgtcccagaggtcctataattgtaaacggttcccttcaattggatctgctcccctcgtacaacagaatatggtatgttcatctccaggaagacatctttgaacacctttgccttgagtgtgtcagcaacacatataccattgtctgagatgccgatgccttgaatttcccaggtcgtcagtgagtcaggcagtgcaacctgcagctggtttcttttgggaacacgatgaacttcccatagccagctctctggaaagtagcttcggatttctgccttcatcactggtaacagggcctttatttggatccttcccaacagcatgcctttgtggtggctttcttttcggatcttatccgcaatagtacaacactcgttgaaggccctgatgcagtgtgggcctatggtcacccgggcaactcgctgctcacaggtttcgtatttgttttctcgggctccatcataacagcatttcttgggcacacggtgtttgtatttagcagcttgttcttccactttctgatgcaggagctgcaggtctctctttggcctgagaatttccttacaagagtcatcgtggtattgggagtcatctgcgtttgcattggtgaggaaggtgagcccagctagatggaatacatctacattgtcacggccaccacctgccccacagcccaggtcactcttgtcatcaaaagcttggaacactctttgcatggcccttttggcttttccccggactccatacacagcgctgtccaccgcagatagtgccacccatgagtctgcttcagtcaccatgtcaagggacacagtttggcctggagaatacacgtctttatctggagacagatggacctggagctggttgccacacttctcctcaatgtttatccagactgcgtcagccaccaattctgctgtctgctcccccgtgactatgtaatagaccaggagccgcgctgaaggaaccatgtcctgtgtcactgggatgtttatattttgataagatgaatagagaagtttctcctttgtgccatactgtacaattttgcctttggataaaatcaagtaattatagtgagttattttgtcaatatatggactcttgggggtgacgataatattcagatattctcccacaagcatgggcttgtagttttcagtccagccaatgtaaatgtaactctggctgagggatgagtatgtaactgcttcatattctttgctggcttgattttcttcgggaagttccggggcatcagttttgacctcaaacttcagtgatgtcacttctgatgggaggttcaccacaaatgaagccactccatcagcagagtgtgtgatgctcctctttggttccaagtcagatgtctcttgattcacattgactgtttgtgccatcagagttactgggacccctcctaccaactgctcgagtgaatccttaacctgtaccttgatggaaaatggaatcccaggcttcaggaaaagaggggtagcgaccaaattcagtgtatagggagagaggacgtatttgatgccaggaatttctgcctcttccgaaaacccacctacaagggcagcaagcactgagttaatgtttccggaaagatgatggcgggctgtaggacatctgtcggtctctgtgctatccat

SEQ ID NO: 9 - RNA, 21 nt, synthetic construct
  misc_feature 1..21: note = Description of Artificial Sequence: Synthetic oligonucleotide
  source 1..21: mol_type = other RNA
tgacaaaataactcactataa

SEQ ID NO: 10 - RNA, 23 nt, synthetic construct
  misc_feature 1..23: note = Description of Artificial Sequence: Synthetic oligonucleotide
  source 1..23: mol_type = other RNA
ttatagtgagttattttgtcaat

SEQ ID NO: 11 - DNA, 25 bp, synthetic construct
  misc_feature 1..25: note = Description of Artificial Sequence: Synthetic oligonucleotide
  modified_base 1..2: mod_base = OTHER; note = uracil
  modified_base 4: mod_base = OTHER; note = uracil
  modified_base 7: mod_base = OTHER; note = uracil
  modified_base 11..12: mod_base = OTHER; note = uracil
  modified_base 14..17: mod_base = OTHER; note = uracil
  modified_base 19: mod_base = OTHER; note = uracil
  modified_base 23: mod_base = OTHER; note = uracil
  source 1..25: mol_type = other DNA
ttatagtgagttattttgtcaattt

SEQ ID NO: 12 - RNA, 21 nt, synthetic construct
  misc_feature 1..21: note = Description of Artificial Sequence: Synthetic oligonucleotide
  source 1..21: mol_type = other RNA
tgacaaaataactcactataa

SEQ ID NO: 13 - DNA, 25 bp, synthetic construct
  misc_feature 1..25: note = Description of Artificial Sequence: Synthetic oligonucleotide
  modified_base 1..2: mod_base = OTHER; note = uracil
  modified_base 4: mod_base = OTHER; note = uracil
  modified_base 7: mod_base = OTHER; note = uracil
  modified_base 11..12: mod_base = OTHER; note = uracil
  modified_base 14..17: mod_base = OTHER; note = uracil
  modified_base 19: mod_base = OTHER; note = uracil
  modified_base 23: mod_base = OTHER; note = uracil
  source 1..25: mol_type = other DNA
ttatagtgagttattttgtcaattt

SEQ ID NO: 14 - RNA, 21 nt, synthetic construct
  misc_feature 1..21: note = Description of Artificial Sequence: Synthetic oligonucleotide
  source 1..21: mol_type = other RNA
tgacaaaataactcactataa

(19) United States
(12) Patent Application Publication
(10) Pub. No.: US 2025/0197855 A1
(43) Pub. Date: Jun. 19, 2025

(54) COMPLEMENT COMPONENT C5 iRNA COMPOSITIONS AND METHODS OF USE

(71) Applicant: Alnylam Pharmaceuticals, Inc., Cambridge, MA (US)

(72) Inventors: Anna Borodovsky, Melrose, MA (US); Andreas J.R. Habenicht, Munich (DE); Changjun Yin, Munich (DE)

(21) Appl. No.: 18/761,504

(22) Filed: Jul. 2, 2024

Related U.S. Application Data
(63) Continuation of application No. 17/204,008, filed on Mar. 17, 2021, now abandoned.
Application No. 17/204,008 is a continuation of application No. PCT/US2019/051430, filed on Sep. 17, 2019.
(60) Provisional application No. 62/732,655, filed on Sep. 18, 2018.

(51) Int. Cl. C12N 15/113

(52) U.S. Cl. CPC C12N 15/113; C12N 2310/11

(57) ABSTRACT

The invention relates to iRNA, e.g., double-stranded ribonucleic acid (dsRNA), compositions targeting the complement component C5 gene, and methods of using such iRNA, e.g., dsRNA, compositions to inhibit expression of C5 and to treat subjects having a complement component C5-associated disease, e.g., Alzheimer's disease, atherosclerosis, or inflammation of the choroid plexus (ChP).

### RELATED APPLICATIONS

This application is a continuation of U.S. patent application Ser. No. 17/204,008, filed Mar. 17, 2021, which is a 35 § U.S.C. 111(a) continuation application which claims the benefit of priority to PCT/US2019/051430, filed on Sep. 17, 2019, which in turn claims the benefit of priority to U.S. Provisional Application No. 62/732,655, filed on Sep. 18, 2018. The entire contents of each of the foregoing applications are incorporated herein by reference.

### SEQUENCE LISTING

The application contains a Sequence Listing which has been submitted electronically in .XML format and is hereby incorporated by reference in its entirety. Said .XML copy, created on Aug. 2, 2024, is named 121301-09203.xml and is 65,698 bytes in size.

### BACKGROUND OF THE INVENTION

Complement was first discovered in the 1890s when it was found to aid or “complement” the killing of bacteria by heat-stable antibodies present in normal serum (Walport, M. J. (2001) N Engl J Med. 344:1058). The complement system consists of more than 30 proteins that are either present as soluble proteins in the blood or are present as membrane-associated proteins. Activation of complement leads to a sequential cascade of enzymatic reactions, known as complement activation pathways, resulting in the formation of the potent anaphylatoxins C3a and C5a that elicit a plethora of physiological responses that range from chemoattraction to apoptosis.

Initially, complement was thought to play a major role in innate immunity where a robust and rapid response is mounted against invading pathogens. However, more recently it has become increasingly evident that complement also plays an important role in adaptive immunity involving T and B cells that help in elimination of pathogens (Dunkelberger J R and Song W C. (2010) Cell Res. 20:34; Molina H, et al. (1996) Proc Natl Acad Sci USA. 93:3357), in maintaining immunologic memory preventing pathogenic re-invasion, and in numerous human pathological states including renal, vascular, neurological, allergic, and infectious disorders (Qu, H, et al. (2009) Mol Immunol. 47:185; Wagner, E. and Frank M M. (2010) Nat Rev Drug Discov. 9:43).

Complement activation is known to occur through three different pathways: alternate, classical, and lectin (FIG. 1), involving proteins that mostly exist as inactive zymogens that are then sequentially cleaved and activated. All pathways of complement activation lead to cleavage of the complement component 5 (C5) molecule generating the anaphylatoxin C5a and, C5b that subsequently forms the terminal complement complex (C5b-9). C5a exerts a predominant pro-inflammatory activity through interactions with the classical G-protein coupled receptor C5aR (CD88) as well as with the non-G protein coupled receptor C5L2 (GPR77), expressed on various immune and non-immune cells. C5b-9 causes cytolysis through the formation of the membrane attack complex (MAC), and sub-lytic MAC and soluble C5b-9 also possess a multitude of non-cytolytic immune functions. These two complement effectors, C5a and C5b-9, generated from C5 cleavage, are key components of the complement system responsible for propagating or initiating pathology in different diseases, including paroxysmal nocturnal hemoglobinuria, rheumatoid arthritis, ischemia-reperfusion injuries and neurodegenerative diseases.

### SUMMARY OF THE INVENTION

The present invention provides iRNA compositions which effect the RNA-induced silencing complex (RISC)-mediated cleavage of RNA transcripts of a C5 gene. The C5 gene may be within a cell, e.g., a cell within a subject, such as a human. The present invention also provides methods for treating a subject having a disorder that would benefit from inhibiting or reducing the expression of a C5 gene, e.g., Alzheimer's disease, atherosclerosis, and inflammation of the choroid plexus (ChP) using iRNA compositions which effect the RNA-induced silencing complex (RISC)-mediated cleavage of RNA transcripts of a C5 gene for inhibiting the expression of a C5 gene.

The invention provides double-stranded ribonucleic acid (dsRNA) agents for use in inhibiting expression of complement component C5 for the prevention or treatment of Alzheimer's disease, atherosclerosis, or inflammation of the choroid plexus (ChP), wherein the dsRNA comprises a sense strand and an antisense strand, wherein the nucleotide sequence of the sense strand comprises 5′-UGACAAAAUAACUCACUAUAA-3′ and the nucleotide sequence of the antisense strand comprises 5′-UUAUAGUGAGUUAUUUUGUCAAU-3′. In certain embodiments, the nucleotide sequence of the sense strand consists of 5′-UGACAAAAUAACUCACUAUAA-3′ and the nucleotide sequence of the antisense strand consists of 5′-UUAUAGUGAGUUAUUUUGUCAAUdTdT-3′.

In certain embodiments, substantially all of the nucleotides of the sense strand and substantially all of the nucleotides of the antisense strand comprise a modification. In certain embodiments, substantially all of the nucleotides of the sense strand comprise a nucleotide modification selected from the group consisting of a 2′-O-methyl modification, a 2′-fluoro modification, and a 3′-terminal deoxy-thymine (dT) nucleotide. In certain embodiments, all of the nucleotides of the sense strand comprise a nucleotide modification selected from the group consisting of a 2′-O-methyl modification, a 2′-fluoro modification, and a 3′-terminal deoxy-thymine (dT) nucleotide. In certain embodiments, substantially all of the nucleotides of the antisense strand comprise a nucleotide modification selected from the group consisting of a 2′-O-methyl modification, a 2′-fluoro modification, and a 3′-terminal deoxy-thymine (dT) nucleotide. In certain embodiments, all of the nucleotides of the antisense strand comprise a nucleotide modification selected from the group consisting of a 2′-O-methyl modification, a 2′-fluoro modification, and a 3′-terminal deoxy-thymine (dT) nucleotide.

In certain embodiments, the sense strand comprises two phosphorothioate internucleotide linkages at the 5′-terminus, and the antisense strand comprises two phosphorothioate internucleotide linkages at the 5′-terminus and two phosphorothioate internucleotide linkages at the 3′-terminus.

In certain embodiments, the dsRNA agent, e.g., the sense strand or the antisense strand of the dsRNA agent, is conjugated to a ligand comprising one or more GalNAc derivatives attached through a branched bivalent or trivalent linker. In one embodiment, the ligand is attached at the 3′-terminus of the sense strand.

In certain embodiments, the dsRNA agents comprise a sense strand and an antisense strand, wherein the nucleotide sequence of the sense strand comprises 5′-usgsAfcAfaAfaUfAfAfcUfcAfcUfaUfaa-3′ and the nucleotide sequence of the antisense strand comprises 5′-usUfsauaGfuGfaGfuuaUfuUfuGfucasasudTdT-3′, wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-O-fluoroadenosine-3′-phosphate, 2′-O-fluorocytidine-3′-phosphate, 2′-O-fluoroguanosine-3′-phosphate, and 2′-O-fluorouridine-3′-phosphate, respectively; dT is a deoxy-thymine; and s is a phosphorothioate linkage; wherein the 3′-end of the sense strand is conjugated to an N-[tris(GalNAc-alkyl)-amidodecanoyl)]-4-hydroxyprolinol (L96) ligand.

In certain embodiments, the nucleotide sequence of the sense strand consists of 5′-usgsAfcAfaAfaUfAfAfcUfcAfcUfaUfaa-3′ and the nucleotide sequence of the antisense strand consists of 5′-usUfsauaGfuGfaGfuuaUfuUfuGfucasasudTdT-3′, wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-O-fluoroadenosine-3′-phosphate, 2′-O-fluorocytidine-3′-phosphate, 2′-O-fluoroguanosine-3′-phosphate, and 2′-O-fluorouridine-3′-phosphate, respectively; dT is a deoxy-thymine; and s is a phosphorothioate linkage; and wherein the 3′-end of the sense strand is conjugated to an N-[tris(GalNAc-alkyl)-amidodecanoyl)]-4-hydroxyprolinol (L96) ligand.

In certain embodiments, the antisense strand comprises a region of complementarity to an mRNA encoding a complement component C5 gene which is 19 to 23 nucleotides in length.

In certain embodiments, each strand is independently 21-30 nucleotides in length. In certain embodiments, the antisense strand is 25 nucleotides in length and the sense strand is 21 nucleotides in length.

In certain embodiments, the ligand is an N-acetylgalactosamine (GalNAc) derivative.

In certain embodiments, the ligand is

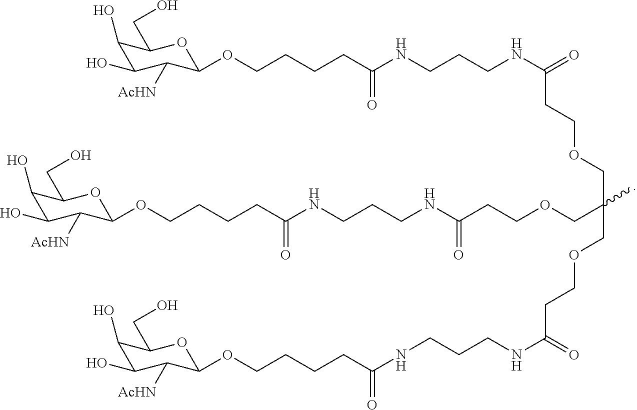 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In certain embodiments, the sense strand is conjugated to the ligand as shown in the following schematic

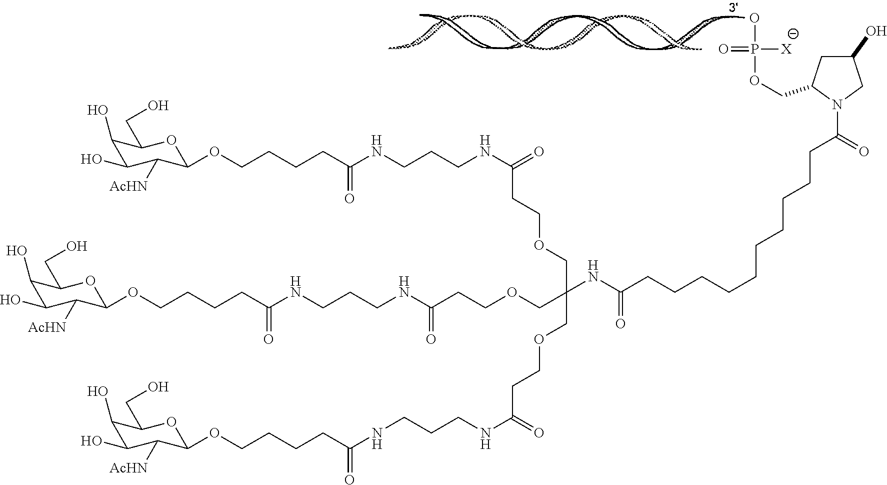

and, wherein X is O or S.

In certain embodiments, X is O.

The invention provides a pharmaceutical composition for prevention or treatment of Alzheimer's disease, atherosclerosis, or inflammation of the choroid plexus (ChP) comprising the dsRNA of the invention.

In certain embodiments, the pharmaceutical composition is formulated for subcutaneous administration. In certain embodiments, the pharmaceutical composition is formulated for administration to a human.

In certain embodiments, the pharmaceutical composition is for the treatment of Alzheimer's disease.

In certain embodiments, the pharmaceutical composition is for the treatment of atherosclerosis.

In certain embodiments, the pharmaceutical composition is for the treatment of inflammation of the choroid plexus (ChP).

In certain embodiments, the dsRNA agent is administered to the subject at a dose of 0.01 mg/kg to 50 mg/kg.

In certain embodiments, the level of complement component C5 in the subject serum is reduced by at least 50%, 55%, 60%, 65%, 70%, 75%, 80%, 85%, or 90%.

The invention provides a method of prevention or treatment of Alzheimer's disease, atherosclerosis, or inflammation of the choroid plexus (ChP) in a subject comprising administration of an effective amount of the dsRNA of the invention or the pharmaceutical composition of the invention to the subject, thereby preventing or treating Alzheimer's disease, atherosclerosis, or inflammation of the choroid plexus (ChP).

In certain embodiments, the dsRNA or the pharmaceutical composition is administered subcutaneously.

In certain embodiments, the subject is human.

In certain embodiments, the disease is Alzheimer's disease.

In certain embodiments, the disease is atherosclerosis.

In certain embodiments, the disease includes inflammation of the choroid plexus (ChP).

In certain embodiments, the dsRNA agent or pharmaceutical composition is administered to the subject at a dose of 0.01 mg/kg to 50 mg/kg.

In certain embodiments, the level of complement component C5 in the subject serum is reduced by at least 50%, 55%, 60%, 65%, 70%, 75%, 80%, 85%, or 90%.

### BRIEF DESCRIPTION OF THE DRAWINGS

FIG. 1 is a schematic of the three complement pathways: alternative, classical and lectin.

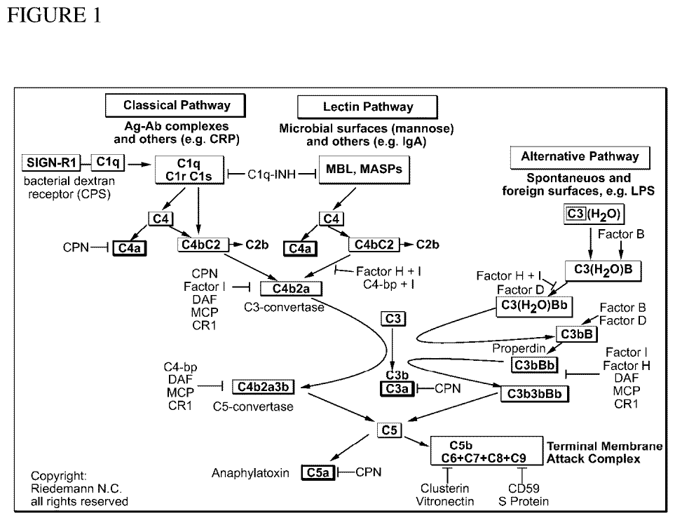

### DETAILED DESCRIPTION OF THE INVENTION

The present invention provides iRNA agents which effect the RNA-induced silencing complex (RISC)-mediated cleavage of RNA transcripts of a complement component C5 gene and the use of those agents for the prevention or treatment of Alzheimer's disease, atherosclerosis, or inflammation of the choroid plexus (ChP).

The data provided herein demonstrate the presence of a classic complement cascade (CCC) activity-regulating C1q-apolipoprotein E (ApoE) complex in diseased choroid plexus, Alzheimer's disease plaques, and atherosclerotic arteries. As ApoE qualifies as a regulator of complement via formation of the C1q-ApoE complex, these data directly tie ApoE to the regulation of the immune system and identify its molecular mechanism of action.

Without being bound by mechanism, it is proposed that the action of ApoE can be described as fine-tuning or tweaking rather than eliminating CCC activity. The CCC is triggered by activation of C1q which can be achieved by multiple mechanisms in diverse sets of physiological and pathophysiological states. The widespread range of C1q activators explains the ubiquitous actions and central position of the CCC to maintain tissue homeostasis. However, inappropriate control of the CCC causes its malfunction, injurious tissue inflammation, and disease. As proposed herein, ApoE is indispensable for CCC regulation as indicated by the marked pathologies of the choroid plexus and of atherosclerosis in ApoE−/− mice and the demonstration that the disease burden can be reduced by C5 siRNA in experimental models as varied as ApoE−/− mice, a model for atherosclerosis, and APPPS1-21 mice, a model for early onset Alzheimer's disease. The salient expression of C1q-ApoE complexes in the choroid plexus, Aβ and neuritic plaques in AD and atherosclerotic arteries suggest multiple therapeutic targets including the complex itself and the downstream constituents of the CCC, as well as their receptors on immune cells.

The two binding partners of the complex, i.e., C1q and ApoE, have previously been viewed as separately acting molecules to perform independent tasks in diverse tissue contexts. Indeed, in addition to regulating complement pathways, various complement constituents act as sometimes beneficial mediators that affect pathways independent of the complement cascades such as inflammasomes and skewing the immune system. Without being bound by mechanism, the data provided herein suggest that at least some pathologies previously thought to reflect the single action of either C1q or ApoE might, in fact, involve the C1q-ApoE complex. Most, if not all, chronic inflammatory diseases are associated with activation of one or more complement pathways and ApoE is induced in response to multiple acute and chronic types of tissue injury. It is suggested, based on the data provided herein, that activated C1q initiates CCC-dependent physiological/beneficial or, if persistent, pathophysiological/injurious inflammation. It follows that the CCC cascade may be targeted by pharmaceuticals at various steps of the CCC cascade. As demonstrated herein, C5-directed siRNA treatment reduced choroid plexus inflammation and diminished the macrophage load and plaque sizes of atherosclerotic intima lesions in ApoE−/− mice in the absence of the endogenous CCC regulator, i.e. in ApoE−/− mice. In addition, in ApoE-sufficient mice, C5 siRNA reduced AD plaque-associated (disease-associated) microglia (DAMs), small and intermediate-sized Aβ plaques, and neuritic plaque-associated lysosomal associated membrane protein 1 (LAMP1).

Alzheimer's disease and atherosclerosis share risk factors while the second most common form of dementia, i.e., vascular dementia, has been closely related to late onset Alzheimer's disease (LOAD). The incidence of Alzheimer's disease is greatly enhanced in patients with atherosclerosis consistent with common mechanisms of disease progression. It is proposed herein that the C1q-ApoE complex forms an active disease-relevant regulatory module which is consistent with the frequent occurrence of autoimmune diseases or immune deficiencies in patients afflicted with genetic absence or loss of function mutations in C1q, C2, C4, and other components of the CCC and the identification of both complement and ApoE as major players in LOAD.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise.

As used herein, “complement component C5,” used interchangeably with the term “C5” refers to the well-known gene and polypeptide, also known in the art as CPAMD4, anaphtlatoxin C5a analog, hemolytic complement (Hc), and complement C5. The sequence of a human C5 mRNA transcript can be found at, for example, GenBank Accession No. GI:38016946 (NM_001735.2; SEQ ID NO:1). The sequence of rhesus C5 mRNA can be found at, for example, GenBank Accession No. GI:297270262 (XM_001095750.2; SEQ ID NO:2). The sequence of mouse C5 mRNA can be found at, for example, GenBank Accession No. GI:291575171 (NM_010406.2; SEQ ID NO:3). The sequence of rat C5 mRNA can be found at, for example, GenBank Accession No. GI:392346248 (XM_345342.4; SEQ ID NO:4). Additional examples of C5 mRNA sequences are readily available using publicly available databases.

The term “C5,” as used herein, also refers to naturally occurring DNA sequence variations of the complement component C5 gene, such as a single nucleotide polymorphism in the C5 gene. Numerous SNPs within the C5 gene have been identified and may be found at, for example, NCBI dbSNP (see, e.g., ncbi.nlm.nih.gov/snp). Non-limiting examples of SNPs within the C5 gene may be found at, NCBI dbSNP Accession Nos. rs121909588 and rs 121909587.

As used herein, “target sequence” refers to a contiguous portion of the nucleotide sequence of an mRNA molecule formed during the transcription of a C5 gene, including mRNA that is a product of RNA processing of a primary transcription product. The target portion of the sequence is at least long enough to serve as a substrate for iRNA-directed cleavage at or near that portion of the nucleotide sequence of an mRNA molecule formed during the transcription of a C5 gene.

The target sequence may be from about 19-30 nucleotides in length, e.g., 19-30, 19-25, 19-23, 19-21, 21-25, or 21-23 nucleotides in length. Ranges and lengths intermediate to the above recited ranges and lengths are also contemplated to be part of the invention.

As used herein, the term “strand comprising a sequence” refers to an oligonucleotide comprising a chain of nucleotides that is described by the sequence referred to using the standard nucleotide nomenclature.

“G,” “C,” “A,” “T” and “U” each generally stand for a nucleotide that contains guanine, cytosine, adenine, thymidine and uracil as a base, respectively. However, it will be understood that the term “ribonucleotide” or “nucleotide” can also refer to a modified nucleotide, as further detailed below, or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

The terms “iRNA”, “RNAi agent,” “iRNA agent,”, “RNA interference agent” as used interchangeably herein, refer to an agent that contains RNA as that term is defined herein, and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., inhibits, the expression of C5 in a cell, e.g., a cell within a subject, such as a mammalian subject.

In one embodiment, an “iRNA” for use in the compositions, uses, and methods of the invention is a double-stranded RNA and is referred to herein as a “double stranded RNAi agent,” “double-stranded RNA (dsRNA) molecule,” “dsRNA agent,” or “dsRNA”. The term “dsRNA”, refers to a complex of ribonucleic acid molecules, having a duplex structure comprising two anti-parallel and substantially complementary nucleic acid strands, referred to as having “sense” and “antisense” orientations with respect to a target RNA, i.e., a C5 gene. In some embodiments of the invention, a double-stranded RNA (dsRNA) triggers the degradation of a target RNA, e.g., an mRNA, through a post-transcriptional gene-silencing mechanism referred to herein as RNA interference or RNAi.

The duplex region may be of any length that permits specific degradation of a desired target RNA through a RISC pathway, and is, in some embodiments, 19-21 base pairs in length, preferably 21 base pairs in length.

In general, the majority of nucleotides of each strand of a dsRNA molecule are ribonucleotides, but as described in detail herein, each or both strands can also include one or more non-ribonucleotides, e.g., a deoxyribonucleotide or a modified nucleotide. In addition, as used in this specification, an “RNAi agent” may include ribonucleotides with chemical modifications; an RNAi agent may include substantial modifications at multiple nucleotides. As used herein, the term “modified nucleotide” refers to a nucleotide having, independently, a modified sugar moiety, a modified internucleotide linkage, or a modified nucleobase. Thus, the term modified nucleotide encompasses substitutions, additions or removal of, e.g., a functional group or atom, to internucleoside linkages, sugar moieties, or nucleobases. The modifications suitable for use in the agents of the invention include all types of modifications disclosed herein or known in the art. Any such modifications, as used in a siRNA type molecule, are encompassed by “RNAi agent” for the purposes of this specification and claims.

As used herein, the term “nucleotide overhang” refers to at least one unpaired nucleotide that protrudes from the duplex structure of an iRNA, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA.

“Blunt” or “blunt end” means that there are no unpaired nucleotides at that end of the double stranded RNAi agent, i.e., no nucleotide overhang. A “blunt ended” RNAi agent is a dsRNA that is double-stranded over its entire length, i.e., no nucleotide overhang at either end of the molecule. The RNAi agents of the invention include RNAi agents with nucleotide overhangs at one end (i.e., agents with one overhang and one blunt end) or with nucleotide overhangs at both ends.

The term “antisense strand” or “guide strand” refers to the strand of an iRNA, e.g., a dsRNA, which includes a region that is substantially complementary to a target sequence, e.g., a C5 mRNA. As used herein, the term “region of complementarity” refers to the region on the antisense strand that is substantially complementary to a sequence, for example a target sequence, e.g., a C5 nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- or 3′-terminus of the iRNA.

The term “sense strand” or “passenger strand” as used herein, refers to the strand of an iRNA that includes a region that is substantially complementary to a region of the antisense strand as that term is defined herein.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an iRNA, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. Where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogstein base pairing.

The terms “complementary” and “fully complementary” herein can be used with respect to the base matching between the sense strand and the antisense strand of a dsRNA, or between the antisense strand of an iRNA agent and a target sequence, as will be understood from the context of their use.

As used herein, a “subject” is an animal, such as a mammal, including a primate (such as a human, a non-human primate, e.g., a monkey, and a chimpanzee), a non-primate (such as a cow, a pig, a horse, a rabbit, a sheep, a hamster, a guinea pig, a cat, a dog, a rat, or a mouse). In an embodiment, the subject is a human, such as a human being treated or assessed for Alzheimer's disease, artherosclerosis, or inflammation of the choroid plexus.

As used herein, the terms “treating” or “treatment” refer to a beneficial or desired result including, but not limited to, alleviation of one or more signs or symptoms associated with Alzheimer's disease, artherosclerosis, or inflammation of the choroid plexus. “Treatment” can also mean prolonging survival as compared to expected survival in the absence of treatment.

The term “lower” in the context of the level of a complement component C5 in a subject or a disease marker, sign, or symptom refers to a statistically significant decrease in such level. The decrease can be, for example, at least 50%, 60%, 70%, 80%, 90%, or more and is preferably down to a level accepted as within the range of normal for an individual without such disorder.

As used herein, “prevention” or “preventing,” when used in reference to Alzheimer's disease, artherosclerosis, or inflammation of the choroid plexus, refers to a reduction in the likelihood that a subject will develop a symptom or sign associated with Alzheimer's disease, artherosclerosis, or inflammation of the choroid plexus. The likelihood of developing a atherosclerosis is reduced, for example, when an individual having one or more risk factors for atherosclerosis either fails to develop artherosclerosis or develops atherosclerosis with less severity relative to a population having the same risk factors and not receiving treatment as described herein. The failure to develop a disease, disorder or condition, or the reduction in the development of a sign or symptom associated with such a disease, disorder or condition (e.g., by at least about 10% on a clinically accepted scale for that disease or disorder), or the exhibition of delayed symptoms delayed (e.g., by days, weeks, months or years) is considered effective prevention.

### II. iRNAs of the Invention

The present invention provides iRNAs which inhibit the expression of a complement component C5 gene. In one embodiment, the iRNA agent includes double-stranded ribonucleic acid (dsRNA) molecules for inhibiting the expression of a C5 gene in a cell, such as a cell within a subject, e.g., a mammal, such as a human having Alzheimer's disease, atherosclerosis, or inflammation.

The dsRNA molecules for use in the invention for use in inhibiting expression of complement component C5 for the prevention or treatment of Alzheimer's disease, atherosclerosis, or inflammation of the choroid plexus, wherein the dsRNA comprises a sense strand and an antisense strand comprising the nucleotide sequences 5′-UGACAAAAUAACUCACUAUAA-3′ and 5′-UUAUAGUGAGUUAUUUUGUCAAU-3′, respectively. In certain embodiments, the nucleotide sequences sense strand and the antisense strand comprise 5′-UGACAAAAUAACUCACUAUAA-3′ and 5′-UUAUAGUGAGUUAUUUUGUCAAUdTdT-3′.

In the dsRNAs for use in the invention, substantially all, or all, of the nucleotides of the sense strand and the antisense strand comprise a modification. Modified nucleotides selected from a 2′-O-methyl modification, a 2′-fluoro modification, and a 3′-terminal deoxy-thymine (dT) nucleotide. Modifications can also include phosphorothioate modifications, particularly modification of the sense strand to include two phosphorothioate internucleotide linkages at the 5′-terminus, and modification of the antisense strand to include two phosphorothioate internucleotide linkages at the 5′-terminus and two phosphorothioate internucleotide linkages at the 3′-terminus.

In the dsRNAs for use in the invention, the dsRNA agent may be conjugated to a ligand comprising one or more GalNAc derivatives attached through a branched bivalent or trivalent linker. For example, in one embodiment, the ligand is attached at the 3′-terminus of the sense strand.

Exemplary embodiments of dsRNA agents for use in the invention the nucleotide sequence of the sense strand comprises or consists of the sequence 5′-usgsAfcAfaAfaUfAfAfcUfcAfcUfaUfaa-3′ the nucleotide sequence of the antisense strand comprises or consists of the sequence 5′-usUfsauaGfuGfaGfuuaUfuUfuGfucasasudTdT-3′, wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-O-fluoroadenosine-3′-phosphate, 2′-O-fluorocytidine-3′-phosphate, 2′-O-fluoroguanosine-3′-phosphate, and 2′-O-fluorouridine-3′-phosphate, respectively; dT is a deoxy-thymine; and s is a phosphorothioate linkage; wherein the 3′-end of the sense strand is conjugated to an N-[tris(GalNAc-alkyl)-amidodecanoyl)]-4-hydroxyprolinol (L96) ligand.

In the dsRNAs, the region of complementarity between the antisense strand and an mRNA encoding a complement component C5 gene may be 19 to 23, or 19 to 21 nucleotides in length.

In the dsRNAs, each strand is independently 21-30 nucleotides in length, e.g., the antisense strand is 25 nucleotides in length and the sense strand is 21 nucleotides in length.

In certain embodiments, the ligand is an N-acetylgalactosamine (GalNAc) derivative, e.g.,

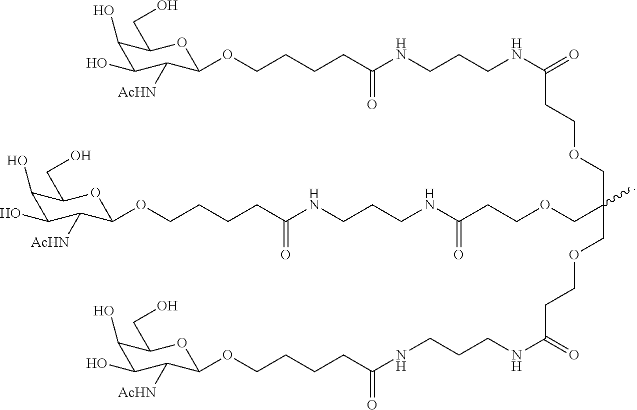 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

and, the sense strand is conjugated to the ligand as shown in the following schematic

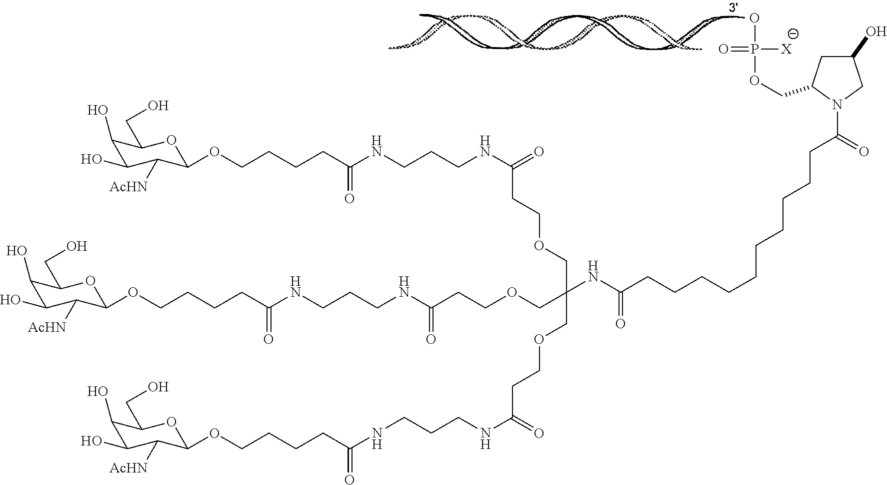

wherein X is O or S, but preferably O.

A dsRNA can be synthesized by standard methods known in the art, e.g., by use of an automated DNA synthesizer, such as are commercially available from, for example, Biosearch, Applied Biosystems, Inc. or by a commercial vendor.

iRNA compounds of the invention may be prepared using a two-step procedure. First, the individual strands of the double-stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Single-stranded oligonucleotides of the invention can be prepared using solution-phase or solid-phase organic synthesis or both.

### III. Modified iRNAs of the Invention

In one embodiment, the RNA of the iRNA of the invention e.g., a dsRNA, is unmodified, and does not comprise, e.g., chemical modifications or conjugations known in the art and described herein. In another embodiment, the RNA of an iRNA of the invention, e.g., a dsRNA, is chemically modified to enhance stability or other beneficial characteristics. In certain embodiments of the invention, substantially all of the nucleotides of an iRNA of the invention are modified. In other embodiments of the invention, all of the nucleotides of an iRNA of the invention are modified. iRNAs of the invention in which “substantially all of the nucleotides are modified” are largely but not wholly modified and can include not more than 5, 4, 3, 2, or 1 unmodified nucleotides.

In a preferred embodiment, the modified dsRNA for use in the pharmaceutical compositions and methods of the invention comprises a modified sense strand and a modified antisense strand. In certain embodiments, the modified sense strand comprises the nucleotide sequence 5′-usgsAfcAfaAfaUfAfAfcUfcAfcUfaUfaaL96-3′ and the modified antisense strand comprises the nucleotide sequence 5′-usUfsauaGfuGfaGfuuaUfuUfuGfucasasudTdT-3′, wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-O-fluoroadenosine-3′-phosphate, 2′-O-fluorocytidine-3′-phosphate, 2′-O-fluoroguanosine-3′-phosphate, and 2′-O-fluorouridine-3′-phosphate, respectively; dT is a deoxy-thymine; s is a phosphorothioate linkage; and L96 is N-[tris(GalNAc-alkyl)-amidodecanoyl)]-4-hydroxyprolinol, also referred to as Hyp-(GalNAc-alkyl)3. In certain embodiments, the modified sense strand consists of the nucleotide sequence 5′-usgsAfcAfaAfaUfAfAfcUfcAfcUfaUfaaL96-3′ and the modified antisense strand consists of the nucleotide sequence 5′-usUfsauaGfuGfaGfuuaUfuUfuGfucasasudTdT-3′, wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-O-fluoroadenosine-3′-phosphate, 2′-O-fluorocytidine-3′-phosphate, 2′-O-fluoroguanosine-3′-phosphate, and 2′-O-fluorouridine-3′-phosphate, respectively; dT is a deoxy-thymine; s is a phosphorothioate linkage; and L96 is N-[tris(GalNAc-alkyl)-amidodecanoyl)]-4-hydroxyprolinol, also referred to as Hyp-(GalNAc-alkyl)3.

### IV. iRNAs Conjugated to Ligands

Another modification of the RNA of an iRNA of the invention involves chemically linking to the RNA one or more ligands, moieties or conjugates that enhance the activity, cellular distribution or cellular uptake of the iRNA.

In one embodiment, the dsRNA agent further comprises a ligand wherein the ligand is an N-acetylgalactosamine (GalNAc) derivative, such as those described in U.S. Patent Publication No. 2009/0239814, the entire contents of which are incorporated herein by reference.

In one embodiment, the ligand is

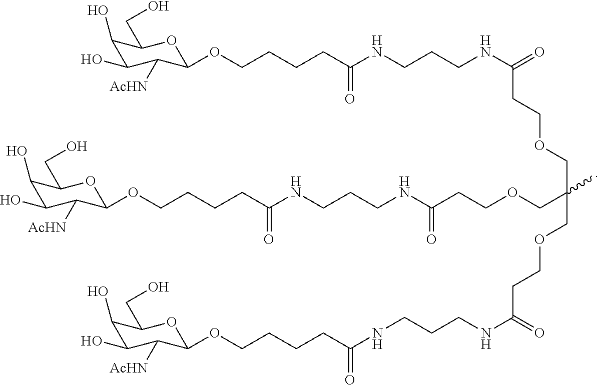 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one embodiment, the dsRNA agent is conjugated to the ligand as shown in the following schematic

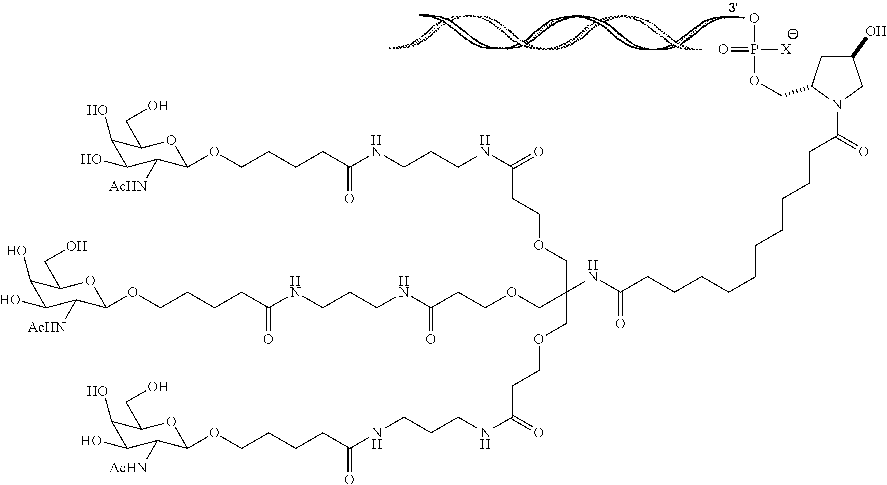

and, wherein X is O or S.

In one embodiment, the X is O.

In some embodiments, the conjugate or ligand described herein can be attached to an iRNA oligonucleotide with various linkers that can be cleavable or non-cleavable.

The term “linker” or “linking group” means an organic moiety that connects two parts of a compound, e.g., covalently attaches two parts of a compound. Linkers are well known in the art and include those described in, for example, U.S. Patent Publication No. 2009/0239814, the entire contents of which are incorporated herein by reference.

In a preferred embodiment, the linking group is a cleavable linking group. A cleavable linking group is one which is sufficiently stable outside the cell, but which upon entry into a target cell is cleaved to release the two parts the linker is holding together. In a preferred embodiment, the cleavable linking group is cleaved at least about 10 times, 20, times, 30 times, 40 times, 50 times, 60 times, 70 times, 80 times, 90 times or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

### V. Delivery of an iRNA of the Invention

The delivery of an iRNA of the invention to a cell e.g., a cell within a subject, such as a human subject (e.g., a subject in need thereof, such as a subject having, suspected of having, or susceptible to Alzheimer's disease, atherosclerosis, or inflammation of the choroid plexus) can be achieved in a number of different ways. For example, delivery may be performed by contacting a cell with an iRNA of the invention either in vitro or in vivo. In vivo delivery may also be performed directly by administering a composition comprising an iRNA, e.g., a dsRNA, to a subject. The dsRNAs of the invention are preferably administered by subcutaneous injection.

### VI. Pharmaceutical Compositions of the Invention

The present invention also includes pharmaceutical compositions and formulations which include the iRNAs of the invention. In one embodiment, provided herein are pharmaceutical compositions containing an iRNA and a pharmaceutically acceptable carrier.

The phrase “pharmaceutically acceptable” is employed herein to refer to those compounds, materials, compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Such agents are well known in the art.

The pharmaceutical compositions containing the iRNA are useful for treating Alzheimer's disease, atherosclerosis, or inflammation of the choroid plexus. Such pharmaceutical compositions are formulated based on the mode of delivery. One example is compositions that are formulated for systemic administration via parenteral delivery, e.g., by subcutaneous (SC) or intravenous (IV) delivery. Formulations for intravenous or subcutaneous delivery of nucleic acid therapeutics are known in the art. The pharmaceutical compositions of the invention may be administered in dosages sufficient to inhibit expression of a C5 gene. In general, a suitable dose of an iRNA of the invention will be in the range of about 0.001 to about 200.0 milligrams per kilogram body weight of the recipient per dose, generally in the range of about 1 to 50 mg per kilogram body weight per dose.

The pharmaceutical composition can be administered, for example, once weekly, once monthly, once every other month, or once every three months.

### VII. Methods of the Invention

The present invention provides therapeutic and prophylactic methods which include administering to a subject having or susceptible to having Alzheimer's disease, atherosclerosis, or choroid plexus inflammation, an iRNA agent or pharmaceutical composition comprising an iRNA agent of the invention.

In one aspect, the present invention provides methods of treating a subject having Alzheimer's disease, atherosclerosis, or choroid plexus inflammation. The treatment methods (and uses) of the invention include administering to the subject, e.g., a human, a therapeutically effective amount of an iRNA agent targeting a C5 gene provided herein or a pharmaceutical composition comprising an iRNA agent targeting a C5 gene provided herein, thereby treating the subject having Alzheimer's disease, atherosclerosis, or choroid plexus inflammation.

In one aspect, the invention provides methods of preventing at least one sign or symptom of Alzheimer's disease, atherosclerosis, or choroid plexus inflammation in a subject having Alzheimer's disease, atherosclerosis, or choroid plexus inflammation. The methods include administering to the subject a prohpylactically effective amount of the dsRNA of the invention, thereby preventing at least one symptom of Alzheimer's disease, atherosclerosis, or choroid plexus inflammation in the subject

“Therapeutically effective amount,” as used herein, is intended to include the amount of the dsRNA of the invention, that, when administered to a subject having Alzheimer's disease, atherosclerosis, or choroid plexus inflammation, is sufficient to effect treatment of the disease (e.g., by diminishing, ameliorating or maintaining the existing disease or one or more signs or symptoms of disease). The “therapeutically effective amount” may vary depending on how the agent is administered, the disease and its severity and the history, age, weight, family history, genetic makeup, the types of preceding or concomitant treatments, if any, and other individual characteristics of the subject to be treated.

“Prophylactically effective amount,” as used herein, is intended to include the amount of the dsRNA of the invention, that, when administered to a subject having Alzheimer's disease, atherosclerosis, or choroid plexus inflammation, but not yet (or currently) experiencing or displaying symptoms of the disease, or a subject at risk of developing Alzheimer's disease, atherosclerosis, or choroid plexus inflammation, is sufficient to prevent or ameliorate the disease or one or more symptoms of the disease. Ameliorating the disease includes slowing the course of the disease or reducing the severity of later-developing disease. The “prophylactically effective amount” may vary depending on the degree of risk of disease, and the history, age, weight, family history, genetic makeup, the types of preceding or concomitant treatments, if any, and other individual characteristics of the patient to be treated.

A “therapeutically effective amount” or “prophylactically effective amount” also includes an amount of the dsRNA of the invention that produces some desired local or systemic effect at a reasonable benefit/risk ratio applicable to any treatment. The dsRNA of the invention employed in the methods of the present invention may be administered in a sufficient amount to produce a reasonable benefit/risk ratio applicable to such treatment.

In yet another aspect, the present invention provides use of the dsRNA of the invention or a pharmaceutical composition comprising the dsRNA of the invention in the manufacture of a medicament for treating a subject, e.g., a subject having Alzheimer's disease, atherosclerosis, or choroid plexus inflammation.

In another aspect, the invention provides uses of the dsRNA of the invention for preventing at least one symptom in a subject suffering from Alzheimer's disease, atherosclerosis, or choroid plexus inflammation.

In a further aspect, the present invention provides uses of an iRNA agent of the invention in the manufacture of a medicament for preventing at least one symptom in a subject suffering from Alzheimer's disease, atherosclerosis, or choroid plexus inflammation.

Administration of the dsRNA according to the methods and uses of the invention may result in a reduction of the severity, signs, symptoms, or markers of Alzheimer's disease, atherosclerosis, or choroid plexus inflammation. By “reduction” in this context is meant a statistically significant decrease in such level. The reduction can be, for example, at least 10%, 20%, 30%, 40%, 50%, 60%, 70%, 80%, 90%, or about 100%.

Efficacy of treatment or prevention of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given iRNA drug or formulation of that drug can also be judged using an experimental animal model for the given disease as known in the art. When using an experimental animal model, efficacy of treatment is evidenced when a statistically significant reduction in a marker or symptom is observed.

### VIII. Animal Models of Alzheimer's Disease, Atherosclerosis, and Choroid Plexus Inflammation

Genetic and induced (e.g., diet induced) animal models of Alzheimer's disease and atherosclerosis are well known in the art. Genetic and induced models of disease may be combined, e.g., feeding a high fat diet to a mouse with a predisposition to atherosclerosis. Some exemplary animal models of these diseases are provided below.

ApoE−/− mice are available from commercial sources and contain a disruption of the endogenous murine ApoE gene (see, e.g., www.taconic.com/transgenic-mouse-model/apoe; www.jax.org/strain/002052). Mice develop normally, but exhibit five times normal serum plasma cholesterol and spontaneous atherosclerotic lesions. Fatty streaks in the proximal aorta are found at 3 months of age. The lesions increase with age and progress to lesions with less lipid but more elongated cells, typical of a more advanced stage of pre-atherosclerotic lesion. Moderately increased triglyceride levels have been reported in mice with this mutation on a mixed C57BL/6 x 129 genetic background. Aged apoE-deficient mice (>17 months) have been shown to develop xanthomatous lesions in the brain consisting mostly of crystalline cholesterol clefts, lipid globules, and foam cells. Smaller xanthomas were seen in the choroid plexus and ventral fornix. Additionally, studies indicate that apoE-deficient mice have altered responses to stress, impaired spatial learning and memory, altered long term potentiation, and synaptic damage. Studies indicate a role for ApoE in immune system regulation, nerve regeneration, and muscle differentiation. Such mice are useful in studying the role of apoE in lipid metabolism, atherogenesis, and nerve injury and to investigate intervention therapies that modify the atherogenic process

ApoE3 knock-in (ApoE3-KI) mice include a knock out of the endogenous mouse ApoE gene with a targeted replacement of the human ApoE3 gene such that the mouse expresses the human ApoE3 gene under the control of the mouse ApoE regulatory sequences (see, e.g., www.taconic.com/transgenic-mouse-model/apoe3). On a normal diet, this model has normal plasma cholesterol and triglyceride levels, but altered relative quantities of different plasma lipoprotein particles, and delayed clearance of vLDL particles. On a high-fat diet, the ApoC3-KI mouse develops abnormal serum lipid profiles and atherosclerotic plaques. The mouse exhibits an increased risk of atherosclerosis and hypercholesterolemia compared with wild type mice on a high fat diet, but not on a normal diet. It is useful for studying the role of human APOE polymorphism in atherosclerosis, lipid metabolism and Alzheimer's disease

Similarly, ApoE4-KI mice are a homozygous for a human APOE4 gene targeted replacement of the endogenous mouse Apoe gene (see, e.g., www.taconic.com/transgenic-mouse-model/apoe4). In humans, the E4 allele is associated with increased plasma cholesterol and a greater risk of coronary artery disease. On a normal diet, this model has normal plasma cholesterol and triglyceride levels, but altered relative quantities of different plasma lipoprotein particles, and delayed clearance of vLDL particles, with only half the clearance rate observed in the APOE3 targeted replacement mice. On a high-fat diet, mice develop abnormal serum lipid profiles and atherosclerotic plaques that are more severe than the APOE3 model, with twice the cholesterol, ApoE, and ApoB-48 levels and larger plaques than the APOE3 model. The mice exhibit an increased risk of atherosclerosis compared with wild type and APOE3 targeted replacement mice. The mouse model is useful for studying the role of human APOE polymorphism in atherosclerosis, lipid metabolism, and Alzheimer's disease.

APPPS1-21 mice (also known as APPPS1 mice) contain human transgenes for both amyloid precursor protein (APP) bearing the Swedish mutation and presenilin 1 (PSEN1) containing an L166P mutation, both under the control of the Thy1 promoter (see, e.g., www.alzforum.org/research-models/appps1). In these mice, expression of the human APP transgene is approximately 3-fold higher than endogenous murine APP. Human Aβ42 is preferentially generated over Aβ40, but levels of both increase with age. In the brain, the Aβ42/Aβ40 decreases with the onset of amyloid deposition. Amyloid plaque deposition starts at approximately six weeks of age in the neocortex. Deposits appear in the hippocampus at about three to four months, and in the striatum, thalamus, and brainstem at four to five months. Phosphorylated tau-positive neuritic processes have been observed in the vicinity of all congophilic amyloid deposits, but no fibrillar tau inclusions are seen.

The high fat fed diet mouse is a well established model for atherosclerosis, diabetes, obesity, hypercholesterolemia, Alzheimer's disease, brain inflammation, and a number of other conditions. The high fat fed model has also been used in combination with genetic models of disease including the ApoE−/− mouse (see, e.g., Li et al., Eur Rev Med Pharmacol Sci. 20:3863-3867, 2016), with mice overexpressing the human APP Swedish mutation (see, e.g., Shie et al., Neuroreport. 13:455-459, 2002), and with mice expressing both the human APP Swedish mutation and the human familial presenilin mutant PS1M146V (see, e.g., Refolo et al., Neurobiol. Dis. 7:321-331, 2000).

Unless otherwise defined, all technical and scientific terms used herein have the same meaning as commonly understood by one of ordinary skill in the art to which this invention belongs. Although methods and materials similar or equivalent to those described herein can be used in the practice or testing of the iRNAs and methods featured in the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1. Materials and Methods

### Source of Reagents

Where the source of a reagent is not specifically given herein, such reagent can be obtained from any supplier of reagents for molecular biology at a quality/purity standard for application in molecular biology.

Sequences of AD-61679

| | Unmodified | Modified |
|---|---|---|
| Strand | nucleotid sequence | nucleotide sequence |
| sense | UGACAAAAUAACUCACUA | usgsAfcAfaAfaUfAfAf |
| | UAA | cUfcAfcUfaUfaaL96 |
| antisense | UUAUAGUGAGUUAUUUUG | usUfsauaGfuGfaGfuua |
| | UCAAU | UfuUfuGfucasasudTdT |

a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively.
Af, Cf, Gf, and Uf are 2′-O-fluoroadenosine-3′-phosphate, 2′-O-fluorocytidine-3′-phosphate, 2′-O-fluoroguanosine-3′-phosphate, and 2′-O-fluorouridine-3′-phosphate, respectively.
dT is a deoxy-thymine.
s is a phosphorothioate linkage.
L96 is N-[tris(GalNAc-alkyl)-amidodecanoyl)]-4-hydroxyprolinol, also referred to as Hyp-(GalNAc-alkyl)3.
Screening and Synthesis of dsRNA Targeted to C5

Design, synthesis, and testing of the AD-61679 duplex are provided in PCT Publication WO 2016/044419, the entire contents of which as they relate to the design, synthesis, and modification of dsRNA agents are incorporated herein by reference.

### Mice

C57BL/6J WT and ApoE−/− mice were purchased from the Jackson Laboratories. WT and ApoE−/− mice were fed a standard rodent chow under pathogen free conditions.

ApoE3 knock-in (ApoE3-KI) and ApoE4-KI mice on C57BL/6 background were purchased from Taconic, USA, and fed either standard rodent chow or fed a high fat cholate-containing diet (Altromin, Germany) containing 15.8% fat, 1.25% cholesterol, and 0.5% sodium cholate. The diet was started at the age of 62 weeks and continued for 16 weeks.

APPPS1-21 mice were studied in collaboration with Mathias Jucker, Hertie Institute for Clinical Brain Research, University of Tübingen. The APPPS1-21 mouse carries double mutations in the Aβ and presenilin genes leading to rapid onset of the pathology of AD.

All animals were maintained and procedures were conducted according to guidelines of the local Animal Use and Care Committees.

C5 siRNA Injection

Mice were randomly separated into two groups. 5 mg/kg C5 siRNA targeting the liver (AD-61679) (20 mg/ml in PBS) or control siRNA targeting luciferase (20 mg/ml in PBS) were administered subcutaneously (s.c.) every two weeks for nine doses starting at the age of 12 weeks for atherosclerosis mouse model; at the age of 6 weeks for Alzheimer's disease mouse model; at the age of 58 weeks for choroid plexus inflammation mouse model. Serum C5 protein levels were determined by ELISA. Complement C5-deficient DAB2 mouse serum was used as negative control for ELISA.

### Human Brain and Choroid Plexus Tissues

All tissues were collected and provided by the Neurobiobank Munich, Ludwig-Maximilians-University (LMU) Munich according to the guidelines of the local ethics committee. ApoE genotype was determined by PCR (Ezway PCR kit, Koma Biotech). AD-related pathologies (neurofibrillary tangles and beta amyloid) were determined according to the guidelines of the Brain Net Europe Consortium (Alafuzoff, I. et al. Brain Pathol 18:484-496, 2008; Alafuzoff, I. et al. Acta Neuropathol. 117:309-320, 2009), and the density of neuritic plaques according to the plaque score modified from CERAD by the National Institute on Aging (Hyman, B. T. et al. Alzheimers Dement. 8:1-13. 2012).

### Human Carotid Artery Tissue

Atherosclerotic plaques were obtained from patients with high-grade carotid artery stenosis (>70%) after carotid endarterectomy. Healthy control carotid arteries were obtained from the Forensic Medicine Institute (type 0-I) (Stary, H. C. Arterioscler Thromb Vasc Biol. 20:1177-1178. 2000). Healthy control arteries comprised all three vessel layers, i.e. the intima, media, and adventitia. Atherosclerotic plaques consisted mainly of the diseased intima resulting from the surgical intervention used for plaque excision (Abbott, A. L. et al. Stroke 46:3288-3301, 2015). The study was performed according to the Guidelines of the World Medical Association Declaration of Helsinki. The local ethics committee of the university hospital where the studies were performed approved the study and written informed consent for permission to be included into the Munich Vascular Biobank was given by all patients.

### Histology and Immunofluorescence

For immunofluorescence staining, tissues were dissected and embedded in Tissue-Tec (Sakura Finetek), frozen in isopentane, and stored at −80° C. 20 μm whole mouse brain coronal sections or one hemisphere of AD mice were prepared according to the mouse brain atlas map.

AD mouse brain sections were stained for Methoxy-X04 (Tocris Bioscience) for Aβ plaque. The total number of Aβ plaques per section or per brain area were quantified using Leica Application Suite (Leica).

The numbers and areas of microglia cells (ibal+/To-Pro-3+ cell) within 30 μm and >30 μm were quantified as described previously (Liu et al., Neuron. 96:1024-1032, 2017). All images were prepared as TIF files by imageJ or Leica LAS-X (V1.2) software.

### Proximity Ligation Assay (PLA)

Protein-protein binding ex vivo was performed by Duolink® PLA kit (DUO92101 SIGMA). Human brain sections, choroid plexus sections, and carotid artery sections were examined by the PLA assay for the presence of C1q/ApoE complexes; Sections were fixed with 4% PFA, then tissue sections were stained with rabbit anti-human ApoE (ab52607, Abcam) and mouse anti-C1q (ab71089, Abcam) with no or one primary antibody as controls. 16 weeks AD (APPPS1-21+/−) brain cortex sections were examined by the PLA assay for the presence of C1q/ApoE complexes, methoxy X04 to outline plaques. C1q-ApoE complexes were observed inside and in the immediate vicinity of Aβ plaque (X04+; X04−), APPPS1-21 mouse brain sections were fixed with 4% PFA, followed with 10 mins Methoxy-X04 (Tocris Bioscience) staining for Aβ plaque. After washing, sections were stained with rabbit anti-mouse ApoE (ab183597, Abcam) and mouse anti-C1q (HM1096BT, Hycult) with no or one primary antibody as controls.

PLA signal was detected by Duolink® PLA kit according to manufacturer's protocol. Leica confocal microscope (SP8, Leica, Germany) equipped with a 100× oil objective (NA 1.4) were used for image. 6 fields per each sample were recorded, 3D reconstructions and the number of PLA signals per volume were performed using LAS-X software package (Leica, v1.2, Germany).

### Example 2. Assessment of Choroid Plexus Lipid Deposits, Inflammation, and Interferon Signatures in Mouse Models of Alzheimer's Disease and Atherosclerosis

The choroid plexus is the major intracranial neuroimmunological interface which produces the cerebrospinal fluid (CSF), forms the blood-CSF barrier, exchanges signals between the brain and the circulation, and is the principal gateway for blood-borne leukocytes to infiltrate the central nervous system in inflammatory and degenerative brain diseases. Lipid deposits, inflammation, and interferon signatures were assessed in the choroid plexus in the ApoE−/− and ApoE3 knock-in (ApoE3-KI) mouse models of atherosclerosis and the ApoE4 knock-in (ApoE4-KI) mouse model of Alzheimer's disease that were either normal chow fed (NC) or a high fat diet fed (HFD).

Similar amounts of lipid accumulated in aged ApoE−/− and HFD ApoE4-KI choroid plexus but no lipid accumulated in NC ApoE4-KI or in NC or HFD ApoE3-KI choroid plexus. Lipid deposits colocalized with leukocytes in ApoE−/− choroid plexuses with the majority of macrophages/dendritic cells (DCs), which were increased in number by a factor of ˜15. Choroid plexus leukocytes, endothelial cells, and epithelial cells accumulated intracellular lipid droplets, as did the ependymal cells lining the ventricle surfaces. The adjacent brain parenchyma underneath the ependymal cells was infiltrated by lipid and leukocytes and exhibited signs of astrocyte activation. Extracellular lipid increased in ApoE−/− versus wild type choroid plexuses by ˜18-fold and also localized at the luminal side of the epithelial cells. High-resolution and transmission electron microscopy (TEM) revealed leukocytes/macrophages in the CSF attached to the microvilli at the abluminal side of the choroid plexus; and some of the intraventricular macrophages accumulated lipid yielding a foam cell-like appearance. These data suggested that macrophages on both sides of the blood-CSF barrier engulf lipid. Since extracellular choroid plexus lipid appeared at the luminal side and the stromal space, the possibility that immunoglobulins (Igs) bind to the lipid droplets was considered. In ApoE−/− choroid plexuses, Igs colocalized with lipid inside the capillary lumen, the stromal space, and the lipid between the epithelial cells but no Ig binding occurred in lipid-free choroid plexuses. These data show that Ig accumulate outside of the blood brain barrier in the choroid plexus on lipid deposits. Bell et al. (Nature. 485:512-516, 2012) previously reported that ApoE-deficiency and transgenic expression of ApoE4 in NC ApoE4-KI mice were afflicted with blood brain barrier breakdown. Igs, used herein as a marker of blood brain barrier breakdown, accumulated in the perivascular space of the lipid-free brain parenchyma of ApoE−/− and NC or HFD ApoE4-KI mice. However, there was no statistically discernable aggravation of blood brain barrier dysfunction as a function of hyperlipidemia.

To delineate differential effects of mouse ApoE vs human ApoE isoforms and the effects of hyperlipidemia on choroid plexus gene expression, laser capture microdissection-based MIAME-compliant microarrays (www.ncbi.nih.gov/geo the NCBI omnibus (GEO); accession: GSE85781) from ChPs of various mouse genotypes that had been maintained on NC or HFD were examined. 241 differentially expressed choroid plexus genes in 6 transcriptomes were identified in gene ontology (GO) terms immune system process, transcription factor binding, cell junction, and ATP binding. In ApoE−/− choroid plexus, the majority (81%) of differentially expressed genes were down-regulated when compared to wild type choroid plexuses; surprisingly, however, 58% ( 7/12) of upregulated genes were interferon (IFN)-related genes with none downregulated. Normal chow fed ApoE4 replacement choroid plexuses further induced (44%, 22/50) IFN-related genes. Multiple two-group comparisons revealed a pronounced ApoE4-specific choroid plexus IFN signature. The biological activities of the IFN-related genes range from regulation of autoimmunity by macrophages and DCs to blood brain barrier integrity including IFN-induced protein with tetratricopeptide repeats 3 and 1 (ifit3, ifit1), ubiquitin-specific peptidase 18 (usp18), guanylate-binding protein 3 (gbp3), interferon-induced protein 44 (ifi44), receptor transporter protein 4 (rtp4), IFN-regulatory factor 7 (irf7), and interferon, alpha-inducible protein 27 like 2A (ifi2712a). These data provided evidence for a detrimental and isoform-specific impact of ApoE4 in choroid plexus homeostasis as choroid plexus IFN has been associated with cognitive decline. Moreover, several genes that were down-regulated in ApoE−/− choroid plexuses were rescued in their ApoE-KI counterparts, indicating phenotypic choroid plexus changes specific for ApoE-deficiency and the ApoE4 genotype. In addition, complement genes were up-regulated in ApoE−/− choroid plexuses.

### Example 3. Complement-Triggered Choroid Plexus Inflammation is Attenuated by C5 siRNA in the Aged ApoE−/− Mouse Model of Atherosclerosis

Oxidation-specific epitopes in extracellular lipids bind Igs and activate complement and complement activation results in surface opsonization by C3b, generation of locally acting anaphylatoxins, i.e. C3a and C5a, and subsequent recruitment of leukocytes and tissue inflammation. It was hypothesized that lipid deposits in ApoE−/− choroid plexuses bind Igs and thereby activate complement. Immunoglobulinis, C3, C3a, and C5 were evident together with lipid in choroid plexuses of ApoE−/− but not in wild type mice. The CCC-initiating C1q molecule and C4 colocalized with choroid plexus lipid deposits. Most complement constituents are produced by the liver and released into the circulation as inactive components or can be produced locally in peripheral tissues. C5 transcripts were below the threshold level in choroid plexus transcriptomes, indicating that choroid plexus C5 was largely serum/liver-derived. To examine whether choroid plexus lipid-triggered CCC activation participates in leukocyte infiltration, liver-derived C5 was specifically targeted for knockdown using the AD-61679 siRNA that selectively binds to the liver asialoglycoprotein receptor. Liver C5 siRNA knockdown led to a large decrease of circulating C5 levels (up to about >95%) without affecting blood lipoprotein concentrations or body weight. Liver-targeted C5 silencing also resulted in substantial decrease of C5 deposits in the choroid plexus and significantly attenuated CD45+ leukocyte−, CD68+ macrophage−/DC−, and CD3+ T-cell infiltration in ApoE−/− choroid plexuses. In contrast, IgG, C4, and C3 deposition were much less affected. These data demonstrate that lipid-triggered complement cascade activation promoted choroid plexus leukocyte infiltration. However, C3 and C4 were present at much lower levels in HFD ApoE4-KI choroid plexuses vs ApoE−/− choroid plexuses despite similar amounts of choroid plexus lipid and respective serum C3 and C5 levels. ApoE colocalized with Igs and C1q. Using an unbiased gene expression microarray, complement-related genes signatures in choroid plexuses were investigated. Six transcripts encoding CCC-specific constituents (c1qa, c1qb, c1qc, c2, c3ar1, C1ra) were identified which were selectively upregulated in choroid plexuses of ApoE−/− as compared to wild type mice. While factor H (alternative complement pathway inhibitor) mRNA was detectable without differences between groups, factor B and MASP1 transcripts were below threshold levels. However, factor H protein accumulation was observed on lipid deposits of both ApoE−/− and HFD ApoE4 choroid plexuses, indicating the presence of a C3b-initiated amplification loop that was inhibited by factor H in both groups of mice. Interestingly, C1qa and C1qc transcripts were rescued in ApoE-KI vs ApoE−/− choroid plexuses and various complement regulators were expressed in ApoE−/− and ApoE-KI choroid plexuses. Taken together, these data revealed pronounced CCC activation in ApoE−/− but not in HFD ApoE3-KI and less in HFD ApoE4-KI mice. In addition, ApoE mRNA ranges were found to be in the top 50 of ˜16,000 genes expressed in wild type choroid plexuses indicating that ApoE is expressed at extraordinarily high levels in normal choroid plexuses.

### Example 4. Choroid Plexus C1q-ApoE Complexes Correlate With Cognitive Decline and are Hallmarks of AD Plaques

Though choroid plexus lipid deposits have not been reported in AD, studies were performed herein to identify pathologies in human AD choroid plexuses that may resemble the pathology of ApoE−/− and HFD ApoE4-KI choroid plexuses. In the studies, 30 age- and gender-matched brains afflicted with various stages of AD-associated pathologies, i.e. Braak & Braak stages for neurofibrillary tangles (NFTs) (Braak et al., Acta Neurophatol. 112:389-404, 2006), Thal phase for Aβ plaque score (Thal et al., Neurology. 58:1791-1800, 2002), and the Consortium to Establish a Registry for Alzheimer's Disease (CERAD) for neuritic plaque (both NFTs and Aβ plaques) burden. 13/30 patients had no signs of dementia (Braak & Braak 0-III, Thal 268 phase 0-5, CERAD stage 0), whereas 17/30 patients exhibited dementia upon clinical neurological examination and showed marked AD pathologies (Braak & Braak IV-VI, Thal phase 1-5, CERAD stage B-C). Surprisingly, 29 of the 30 brains showed various degrees of ChP lipid deposits that were strikingly similar to those found in ApoE−/− and HFD ApoE4-KI choroid plexuses. Notably, demented AD cases revealed higher rates of lipid in choroid plexuses versus non-dementia cases. Moreover, the burden of choroid plexus lipid deposits correlated with all AD neuropathologies and the choroid plexus lipid content especially correlated with ApoE4 allele carriers. Unexpectedly, ApoE3/ApoE3 demented AD cases also had a significantly higher rate of choroid plexus lipid positive areas when compared to ApoE3/ApoE3 non-dementia cases. Choroid plexus lipid colocalized with C1q, ApoE, and complement C3 and C5. Choroid plexus lipid deposits were associated with intraluminal macrophage infiltration, very similar to mouse ApoE−/− choroid plexuses. Factor H protein deposition was observed in both lipid positive and lipid negative choroid plexuses in dementia cases.

C1q-ApoE complex formation in the choroid plexus and brain was evaluated using the proximity ligation assay (PLA) with a resolution power of 10-30 nm, comparable to resonance energy transfer-type technologies and super-resolution stimulated emission depletion (STED) microscopy was applied in parallel. By PLA, it was observed that the C1q-ApoE complex forms in human choroid plexuses in vivo and that its density in ChP lipid-rich areas was higher when compared to lipid-free areas. C1q, Aβ, phosphorylated Tau (pTau), as well as C3 co-localized with ApoE in brains of human AD. C1q-ApoE complexes were also observed in human neuritic plaques. Moreover, Aβ-ApoE complexes but not ApoE-pTau complexes accumulated in AD plaques of demented cases, demonstrating that ApoE binds to Aβ in vivo. These data add to earlier reports that ApoE, C1q, and C3 are detectable in human AD plaques by demonstrating the buildup of the C1q-ApoE and Aβ-ApoE complexes in brains of AD cases with dementia.

These data demonstrated that choroid plexus inflammation may represent an novel unrecognized pathology that is associated with cognitive decline. Administration of C5 siRNA AD-61679 is effective to reduce inflammation in a mouse model of choroid plexus inflammation.

### Example 5. C5 siRNA Reduces Disease-Associated Microglia Cells (DAMs) in a Mouse Model of AD

The APPPS1-21 mouse, which carries double mutations in the Aβ and presenilin genes leading to a rapid onset pathology of AD, was then used to further study C1q-ApoE complex in vivo. High resolution 3D confocal microscopy of C1q-ApoE complexes that had been visualized by the PLA assay revealed that the complexes accumulate inside as well as in the immediate vicinity of methoxy-X04+ Aβ plaques in APPPS1-21 cortexes, i.e. the area of AD plaques that show microglia infiltration. Aβ-ApoE complexes were also observed in APPPS1-21 mouse brains. Somewhat unlike C1q-ApoE complexes, the majority of Aβ-ApoE complexes located inside X04+ Aβ plaque. APPPS1-21 mice were treated with the liver-specific C5 siRNA AD-61679. C5 siRNA treatment significantly reduced serum C5 (about >95%), and the number and density of Aβ-associated microglia cells (about 30%) and of Aβ plaque-associated LAMP1 (about 11%). C5 siRNA AD-61679 also reduced the percentage of small and intermediate-sized plaque volumes (about 30%) though the total plaque load was unchanged. In addition, C1q-ApoE complexes but not Aβ-ApoE complexes were observed in 8 weeks old WT brain cortexes indicating a role of the complex in normal brain homeostasis.

These data demonstrate that administration of C5 siRNA AD-61679 is effective at decreasing signs of AD in the brain of the APPPS1-21 mouse, well-recognized mouse model of AD.

### Example 6. C5 siRNA Reduces Atherosclerosis Inflammation

The data provided herein raised the possibility that other unresolvable human diseases showed similar pathological hallmarks that were identified in ApoE−/− choroid plexuses from mice and humans, and in mouse AD brains. When gene expression signatures were mined in wild type vs ApoE−/− aortas, 9 complement pathway-related transcripts (largely CCC-related) were found to be >2-fold upregulated in ApoE−/− aortas during development of aortic arch atherosclerosis. The impact of CCC activation on early atherosclerosis was supported by a ˜65% decrease in both thoracic and abdominal atherosclerosis by treatment with the C5 siRNA AD-61679, without affecting blood lipid levels, body weight, or blood leukocyte counts.

CCC activation in human carotid atherosclerosis was then evaluated. Five healthy control arteries on autopsy (type 0-I; 3 American Heart Association classification (Stary et al., Arterioscler Thromb Vasc Biol 20:1177-1178, 2000)), six early (type II-III) and nine advanced atherosclerotic plaques (type V-VII) from carotid endarterectomy specimens were stained for CD68+ macrophages/DCs, C1q, ApoE, and C5. CD68+ macrophages, and C1q, ApoE, and C5 protein deposits increased in early and advanced plaques when compared to control arteries. C1q and ApoE co-localized in atherosclerotic plaques as determined by STED microscopy. However, although both C1q and ApoE were colocalized in the uninflamed media layer of ApoE−/− mice, no C1q-ApoE complexes were detectable there. However, the C1q-ApoE complex emerged as a pathological hallmark of atherosclerotic plaques and malondialdehyde-epitopes (MDA2) were observed on the surface of lipid deposits within plaques.

These data demonstrate C1-ApoE complexes are hallmarks of complement activation in human atherosclerosis, administration of C5 siRNA AD-61679 is effective to reduce disease burden at ApoE−/− mice.

### Informal Sequence Listing

>gi|38016946|ref|NM_001735.2| Homo sapiens complement component 5 (C5),
mRNA
SEQ ID NO: 1
TATATCCGTGGTTTCCTGCTACCTCCAACCATGGGCCTTTTGGGAATACTTTGTTTTTTAATCTTCCTGG
GGAAAACCTGGGGACAGGAGCAAACATATGTCATTTCAGCACCAAAAATATTCCGTGTTGGAGCATCTGA
AAATATTGTGATTCAAGTTTATGGATACACTGAAGCATTTGATGCAACAATCTCTATTAAAAGTTATCCT
GATAAAAAATTTAGTTACTCCTCAGGCCATGTTCATTTATCCTCAGAGAATAAATTCCAAAACTCTGCAA
TCTTAACAATACAACCAAAACAATTGCCTGGAGGACAAAACCCAGTTTCTTATGTGTATTTGGAAGTTGT
ATCAAAGCATTTTTCAAAATCAAAAAGAATGCCAATAACCTATGACAATGGATTTCTCTTCATTCATACA
GACAAACCTGTTTATACTCCAGACCAGTCAGTAAAAGTTAGAGTTTATTCGTTGAATGACGACTTGAAGC
CAGCCAAAAGAGAAACTGTCTTAACTTTCATAGATCCTGAAGGATCAGAAGTTGACATGGTAGAAGAAAT
TGATCATATTGGAATTATCTCTTTTCCTGACTTCAAGATTCCGTCTAATCCTAGATATGGTATGTGGACG
ATCAAGGCTAAATATAAAGAGGACTTTTCAACAACTGGAACCGCATATTTTGAAGTTAAAGAATATGTCT
TGCCACATTTTTCTGTCTCAATCGAGCCAGAATATAATTTCATTGGTTACAAGAACTTTAAGAATTTTGA
AATTACTATAAAAGCAAGATATTTTTATAATAAAGTAGTCACTGAGGCTGACGTTTATATCACATTTGGA
ATAAGAGAAGACTTAAAAGATGATCAAAAAGAAATGATGCAAACAGCAATGCAAAACACAATGTTGATAA
ATGGAATTGCTCAAGTCACATTTGATTCTGAAACAGCAGTCAAAGAACTGTCATACTACAGTTTAGAAGA
TTTAAACAACAAGTACCTTTATATTGCTGTAACAGTCATAGAGTCTACAGGTGGATTTTCTGAAGAGGCA
GAAATACCTGGCATCAAATATGTCCTCTCTCCCTACAAACTGAATTTGGTTGCTACTCCTCTTTTCCTGA
AGCCTGGGATTCCATATCCCATCAAGGTGCAGGTTAAAGATTCGCTTGACCAGTTGGTAGGAGGAGTCCC
AGTAACACTGAATGCACAAACAATTGATGTAAACCAAGAGACATCTGACTTGGATCCAAGCAAAAGTGTA
ACACGTGTTGATGATGGAGTAGCTTCCTTTGTGCTTAATCTCCCATCTGGAGTGACGGTGCTGGAGTTTA
ATGTCAAAACTGATGCTCCAGATCTTCCAGAAGAAAATCAGGCCAGGGAAGGTTACCGAGCAATAGCATA
CTCATCTCTCAGCCAAAGTTACCTTTATATTGATTGGACTGATAACCATAAGGCTTTGCTAGTGGGAGAA
CATCTGAATATTATTGTTACCCCCAAAAGCCCATATATTGACAAAATAACTCACTATAATTACTTGATTT
TATCCAAGGGCAAAATTATCCACTTTGGCACGAGGGAGAAATTTTCAGATGCATCTTATCAAAGTATAAA
CATTCCAGTAACACAGAACATGGTTCCTTCATCCCGACTTCTGGTCTATTACATCGTCACAGGAGAACAG
ACAGCAGAATTAGTGTCTGATTCAGTCTGGTTAAATATTGAAGAAAAATGTGGCAACCAGCTCCAGGTTC
ATCTGTCTCCTGATGCAGATGCATATTCTCCAGGCCAAACTGTGTCTCTTAATATGGCAACTGGAATGGA
TTCCTGGGTGGCATTAGCAGCAGTGGACAGTGCTGTGTATGGAGTCCAAAGAGGAGCCAAAAAGCCCTTG
GAAAGAGTATTTCAATTCTTAGAGAAGAGTGATCTGGGCTGTGGGGCAGGTGGTGGCCTCAACAATGCCA
ATGTGTTCCACCTAGCTGGACTTACCTTCCTCACTAATGCAAATGCAGATGACTCCCAAGAAAATGATGA
ACCTTGTAAAGAAATTCTCAGGCCAAGAAGAACGCTGCAAAAGAAGATAGAAGAAATAGCTGCTAAATAT
AAACATTCAGTAGTGAAGAAATGTTGTTACGATGGAGCCTGCGTTAATAATGATGAAACCTGTGAGCAGC
GAGCTGCACGGATTAGTTTAGGGCCAAGATGCATCAAAGCTTTCACTGAATGTTGTGTCGTCGCAAGCCA
GCTCCGTGCTAATATCTCTCATAAAGACATGCAATTGGGAAGGCTACACATGAAGACCCTGTTACCAGTA
AGCAAGCCAGAAATTCGGAGTTATTTTCCAGAAAGCTGGTTGTGGGAAGTTCATCTTGTTCCCAGAAGAA
AACAGTTGCAGTTTGCCCTACCTGATTCTCTAACCACCTGGGAAATTCAAGGCGTTGGCATTTCAAACAC
TGGTATATGTGTTGCTGATACTGTCAAGGCAAAGGTGTTCAAAGATGTCTTCCTGGAAATGAATATACCA
TATTCTGTTGTACGAGGAGAACAGATCCAATTGAAAGGAACTGTTTACAACTATAGGACTTCTGGGATGC
AGTTCTGTGTTAAAATGTCTGCTGTGGAGGGAATCTGCACTTCGGAAAGCCCAGTCATTGATCATCAGGG
CACAAAGTCCTCCAAATGTGTGCGCCAGAAAGTAGAGGGCTCCTCCAGTCACTTGGTGACATTCACTGTG
CTTCCTCTGGAAATTGGCCTTCACAACATCAATTTTTCACTGGAGACTTGGTTTGGAAAAGAAATCTTAG
TAAAAACATTACGAGTGGTGCCAGAAGGTGTCAAAAGGGAAAGCTATTCTGGTGTTACTTTGGATCCTAG
GGGTATTTATGGTACCATTAGCAGACGAAAGGAGTTCCCATACAGGATACCCTTAGATTTGGTCCCCAAA
ACAGAAATCAAAAGGATTTTGAGTGTAAAAGGACTGCTTGTAGGTGAGATCTTGTCTGCAGTTCTAAGTC
AGGAAGGCATCAATATCCTAACCCACCTCCCCAAAGGGAGTGCAGAGGCGGAGCTGATGAGCGTTGTCCC
AGTATTCTATGTTTTTCACTACCTGGAAACAGGAAATCATTGGAACATTTTTCATTCTGACCCATTAATT
GAAAAGCAGAAACTGAAGAAAAAATTAAAAGAAGGGATGTTGAGCATTATGTCCTACAGAAATGCTGACT
ACTCTTACAGTGTGTGGAAGGGTGGAAGTGCTAGCACTTGGTTAACAGCTTTTGCTTTAAGAGTACTTGG
ACAAGTAAATAAATACGTAGAGCAGAACCAAAATTCAATTTGTAATTCTTTATTGTGGCTAGTTGAGAAT
TATCAATTAGATAATGGATCTTTCAAGGAAAATTCACAGTATCAACCAATAAAATTACAGGGTACCTTGC
CTGTTGAAGCCCGAGAGAACAGCTTATATCTTACAGCCTTTACTGTGATTGGAATTAGAAAGGCTTTCGA
TATATGCCCCCTGGTGAAAATCGACACAGCTCTAATTAAAGCTGACAACTTTCTGCTTGAAAATACACTG
CCAGCCCAGAGCACCTTTACATTGGCCATTTCTGCGTATGCTCTTTCCCTGGGAGATAAAACTCACCCAC
AGTTTCGTTCAATTGTTTCAGCTTTGAAGAGAGAAGCTTTGGTTAAAGGTAATCCACCCATTTATCGTTT
TTGGAAAGACAATCTTCAGCATAAAGACAGCTCTGTACCTAACACTGGTACGGCACGTATGGTAGAAACA
ACTGCCTATGCTTTACTCACCAGTCTGAACTTGAAAGATATAAATTATGTTAACCCAGTCATCAAATGGC
TATCAGAAGAGCAGAGGTATGGAGGTGGCTTTTATTCAACCCAGGACACAATCAATGCCATTGAGGGCCT
GACGGAATATTCACTCCTGGTTAAACAACTCCGCTTGAGTATGGACATCGATGTTTCTTACAAGCATAAA
GGTGCCTTACATAATTATAAAATGACAGACAAGAATTTCCTTGGGAGGCCAGTAGAGGTGCTTCTCAATG
ATGACCTCATTGTCAGTACAGGATTTGGCAGTGGCTTGGCTACAGTACATGTAACAACTGTAGTTCACAA
AACCAGTACCTCTGAGGAAGTTTGCAGCTTTTATTTGAAAATCGATACTCAGGATATTGAAGCATCCCAC
TACAGAGGCTACGGAAACTCTGATTACAAACGCATAGTAGCATGTGCCAGCTACAAGCCCAGCAGGGAAG
AATCATCATCTGGATCCTCTCATGCGGTGATGGACATCTCCTTGCCTACTGGAATCAGTGCAAATGAAGA
AGACTTAAAAGCCCTTGTGGAAGGGGTGGATCAACTATTCACTGATTACCAAATCAAAGATGGACATGTT
ATTCTGCAACTGAATTCGATTCCCTCCAGTGATTTCCTTTGTGTACGATTCCGGATATTTGAACTCTTTG
AAGTTGGGTTTCTCAGTCCTGCCACTTTCACAGTGTACGAATACCACAGACCAGATAAACAGTGTACCAT
GTTTTATAGCACTTCCAATATCAAAATTCAGAAAGTCTGTGAAGGAGCCGCGTGCAAGTGTGTAGAAGCT
GATTGTGGGCAAATGCAGGAAGAATTGGATCTGACAATCTCTGCAGAGACAAGAAAACAAACAGCATGTA
AACCAGAGATTGCATATGCTTATAAAGTTAGCATCACATCCATCACTGTAGAAAATGTTTTTGTCAAGTA
CAAGGCAACCCTTCTGGATATCTACAAAACTGGGGAAGCTGTTGCTGAGAAAGACTCTGAGATTACCTTC
ATTAAAAAGGTAACCTGTACTAACGCTGAGCTGGTAAAAGGAAGACAGTACTTAATTATGGGTAAAGAAG
CCCTCCAGATAAAATACAATTTCAGTTTCAGGTACATCTACCCTTTAGATTCCTTGACCTGGATTGAATA
CTGGCCTAGAGACACAACATGTTCATCGTGTCAAGCATTTTTAGCTAATTTAGATGAATTTGCCGAAGAT
ATCTTTTTAAATGGATGCTAAAATTCCTGAAGTTCAGCTGCATACAGTTTGCACTTATGGACTCCTGTTG
TTGAAGTTCGTTTTTTTGTTTTCTTCTTTTTTTAAACATTCATAGCTGGTCTTATTTGTAAAGCTCACTT
TACTTAGAATTAGTGGCACTTGCTTTTATTAGAGAATGATTTCAAATGCTGTAACTTTCTGAAATAACAT
GGCCTTGGAGGGCATGAAGACAGATACTCCTCCAAGGTTATTGGACACCGGAAACAATAAATTGGAACAC
CTCCTCAAACCTACCACTCAGGAATGTTTGCTGGGGCCGAAAGAACAGTCCATTGAAAGGGAGTATTACA
AAAACATGGCCTTTGCTTGAAAGAAAATACCAAGGAACAGGAAACTGATCATTAAAGCCTGAGTTTGCTT
TCAAAAAAAAAAAAAAAAAA
>gi|297270262|ref|XM_001095750.2| PREDICTED: Macaca mulatta complement component 5
(C5), mRNA
SEQ ID NO: 2
CATGATTTCCTGCTACCTCCAACCATGGGCCTTTTGGGAATACTTTGTTTTTTAATCTTCCTGGGAAAAA
CTTGGGGACAGGAGCAAACATATGTCATTTCAGCACCAAAAATATTCCGTGTTGGAGCATCTGAAAACAT
TGTGATTCAAGTTTATGGATACACTGAAGCATTTGATGCAACAATCTCTATTAAAAGTTATCCTGATAAA
AAATTTAGTTACTCCTCAGGCCATGTTCATTTATCCTCAGAGAATAAATTCCAAAACTCGGCAGTCTTAA
CAATACAACCAAAACAATTACCTGGAGGACAAAACCAAGTTTCTTATGTGTATTTGGAAGTTGTATCAAA
GCATTTTTCAAAATCAAAAAAAATTCCAATAACCTATGACAATGGATTTCTCTTCATTCATACAGACAAA
CCTGTTTATACTCCAGACCAATCAGTAAAGGTTAGAGTTTATTCGTTGAATGATGACTTGAAGCCAGCCA
AAAGAGAAACTGTCTTAACTTTCATAGATCCTGAAGGATCAGAAATTGACATGGTAGAAGAAATTGATCA
TATTGGAATTATCTCTTTTCCTGACTTCAAGATTCCGTCTAATCCTAGATATGGTATGTGGATGATCCAG
GCTAAATATAAAGAGGACTTTTCAACAACTGGAACTGCATTTTTTGAAGTTAAAGAATATGTCTTGCCAC
ATTTTTCTGTCTCAGTAGAACCAGAAAGTAATTTCATTGGTTATAAGAACTTTAAGAATTTTGAAATTAC
TATAAAAGCAAGATATTTTTATAATAAAGTAGTCACTGAGGCTGATGTTTATATCACATTTGGAATAAGA
GAAGACTTAAAAGATGATCAAAAAGAAATGATGCAAACAGCAATGCAAAACACAATGTTGATAAATGGAA
TTGCTCAAGTCACATTTGATTCTGAAACAGCAGTCAAAGAACTGTCATACTACAGTTTAGAAGATTTAAA
CAACAAGTACCTTTATATTGCTGTAACAGTCATAGAGTCTACAGGTGGATTTTCTGAAGAGGCAGAAATA
CCTGGCATCAAATATGTCCTCTCTCCCTACAAACTGAATTTGGTTGCTACTCCTCTTTTCCTGAAGCCTG
GGATTCCATATTCCATCAAGGTGCAGGTTAAAGATGCGCTTGACCAGTTGGTAGGAGGGGTCCCAGTAAC
ACTGAATGCACAAACAATTGATGTCAACCAAGAGACATCTGACTTGGAGCCAAGGAAAAGTGTAACACGT
GTTGATGATGGAGTAGCTTCGTTTGTGGTTAATCTCCCATCTGGAGTGACGGTGCTGGAGTTTAATGTCA
AAACTGATGCTCCAGATCTTCCAGACGAAAATCAGGCCAGGGAAGGTTACCGAGCAATAGCATACTCATC
TCTCAGCCAAAGTTACCTTTATATCGATTGGACTGATAACCACAAGGCTTTGCTAGTGGGAGAATATTTG
AATATTATTGTTACCCCCAAAAGCCCATATATTGACAAAATAACTCACTATAATTACTTGATTTTATCCA
AGGGCAAAATTATCCACTTTGGCACAAGGGAGAAACTTTCAGATGCATCTTATCAAAGTATAAACATTCC
AGTAACGCAGAACATGGTTCCTTCATCCCGACTCCTGGTCTATTACATCGTCACAGGAGAGCAGACAGCA
GAATTAGTGTCTGATTCAGTCTGGTTAAATATTGAAGAAAAATGTGGCAACCAGCTCCAGGTTCATCTGT
CTCCTGATGCAGATACATATTCTCCAGGCCAAACTGTGTCTCTTAATATGGTAACTGGGATGGATTCCTG
GGTGGCATTAACAGCAGTGGACAGCGCTGTGTATGGAGTCCAAAGAAGAGCCAAAAAGCCCTTGGAAAGA
GTATTTCAATTCTTAGAGAAGAGTGATCTGGGCTGTGGGGCAGGTGGTGGCCTCAACAATGCCAATGTGT
TCCACCTAGCTGGACTTACCTTCCTCACTAATGCAAATGCAGATGACTCCCAAGAAAATGATGAACCTTG
TAAAGAAATTATCAGGCCAAGAAGAATGCTACAAGAGAAGATAGAAGAAATAGCTGCTAAATATAAACAT
TTAGTAGTGAAGAAATGTTGTTACGATGGAGTCCGTATTAATCATGATGAAACCTGTGAGCAGCGAGCTG
CACGGATTAGTGTAGGGCCGAGATGCGTCAAAGCTTTCACTGAATGTTGTGTCGTCGCAAGCCAGCTCCG
TGCTAATAACTCTCATAAAGACTTGCAATTGGGAAGGCTACACATGAAGACCCTGTTACCAGTAAGCAAG
CCAGAAATTCGGAGTTATTTTCCAGAAAGCTGGTTATGGGAAGTTCATCTTGTTCCCAGAAGAAAACAGT
TGCAGTTTGCCCTACCTGATTCTGTAACTACCTGGGAAATTCAAGGTGTTGGCATTTCAAACAGTGGTAT
ATGTGTTGCTGATACTATTAAGGCAAAGGTGTTCAAAGATGTCTTCCTGGAAATGAATATACCATATTCT
GTTGTACGAGGAGAACAGGTCCAGTTGAAAGGAACTGTTTACAACTATAGGACTTCTGGGATGCAGTTCT
GTGTTAAAATGTCTGCTGTGGAGGGAATCTGCACTTCAGAAAGCCCAGTCATTGATCATCAGGGCACAAA
GTCCTCCAAATGTGTGCGACAGAAAGTAGAGGGCTCCTCTAATCACTTGGTGACCTTTACTGTGCTTCCT
CTGGAAATTGGCCTTCAGAACATCAATTTCTCACTGGAGACTTCGTTTGGAAAAGAAATCTTAGTAAAAT
CGTTACGAGTGGTGCCAGAAGGTGTCAAAAGGGAAAGCTATTCTGGTATTACTTTGGATCCTAGGGGTAT
TTATGNNNNNNNNNNNNNNNNNNNNCGAAAGGAGTTCCCATACAGGATACCATTAGATTTGGTCCCCAAA
ACAGAAATCAAAAGGATTTTGAGTGTAAAAGGACTGCTTGTAGGTGAGATCTTGTCTGCAGTTCTAAGTC
GGGAAGGCATCAATATCCTAACCCACCTCCCCAAAGGGAGTGCAGAGGCGGAGCTGATGAGCGTTGTCCC
AGTATTCTATGTTTTTCACTACCTGGAAACAGGAAATCATTGGAACATTTTTCATTCCGACCCATTAATT
GAAAAGCGGAACCTGGAGAAAAAATTAAAAGAAGGGATGGTGAGCATTATGTCCTACAGAAATGCTGACT
ATTCTTACAGCGTGTGGAAGGGTGGCAGTGCTAGCACTTGGTTAACAGCTTTTGCTTTAAGAGTACTTGG
ACAAGTACATAAATATGTAGAGCAGAACCAAAATTCAATATGTAATTCTTTATTGTGGCTGGTTGAGAAT
TATCAGTTAGATAATGGATCCTTCAAGGAAAATTCACAGTATCAACCAATAAAATTACAGAAAATCAACA
CAGCTCTAATTAAAGCTGACACCTTTCTGCTTGAAAATACACTGCCAGCCCAGAGCACCTTTACATTGGC
CATTTCTGCCTATGCTCTTTCCCTGGGAGATAAAACTCACCCACAGTTTTGTTCAATTGTTTCAGCTTTG
AAGAGAGAAGCTTTGGTTAAAGGTAATCCACCCATTTATCGTTTTTGGAAAGACAGTCTTCAACATAAAG
ACAGCTCTGTACCTAACACTGGTACAGCACGTATGGTAGAAACAACTGCCTATGCTTTACTCACCAGTCT
GAACTTGAAAGACATAAATTATGTTAACCCAATCATCAAATGGCTATCAGAAGAGCAGAGGTATGGAGGT
GGCTTTTATTCAACCCAGGACACAATCAATGCCATCGAGGGCCTGACAGAATATTCACTCCTGGTTAAAC
AGCTCCGCTTGAATATGGACATCGATGTTGCTTACAAGCATAAAGGTCCCTTACATAATTATAAAATGAC
AGACAAGAATTTCCTTGGGAGGCCAGTAGAGGTGCTTCTCAATGATGACCTCGTTGTCAGTACAGGATTT
GGCAGTGGCTTGGCTACGGTACATGTAACAACTGTAGTTCACAAAACCAGTACCTCTGAGGAAGTTTGCA
GCTTTTATTTGAAAATTGATACTCAGGATATTGAAGCATCCCACTACAGAGGCTACGGAAACTCTGATTA
CAAACGCATAGTAGCATGTGCCAGCTACAAGCCCAGCAAGGAAGAATCATCTTCTGGATCCTCTCATGCA
GTGATGGACATCTCCTTGCCTACTGGAATCAATGCAAATGAAGAAGACTTAAAAGCTCTTGTGGAAGGGG
TGGATCAGCTATTCACTGATTACCAAATAAAAGATGGACATGTTATTCTGCAACTGAATTCGATCCCCTC
CAGTGATTTCCTTTGTGTACGATTCCGGATTTTTGAACTCTTTGAAGTTGGGTTTCTTAGTCCTGCCACT
TTCACAGTGTATGAATACCACAGACCAGATAAACAGTGTACCATGTTTTATAGCACTTCCAATATCAAAA
TTCAGAAAGTCTGTGAAGGAGCCACGTGCAAGTGTATAGAAGCTGATTGTGGGCAAATGCAGAAAGAATT
GGATCTGACAATCTCTGCAGAGACTAGAAAACAAACAGCATGTAACCCAGAGATTGCATATGCTTATAAA
GTTATCATCACATCCATCACTACAGAAAATGTTTTTGTCAAGTACAAGGCAACCCTTCTGGATATCTACA
AAACTGGGGAAGCTGTTGCTGAAAAAGACTCTGAAATCACCTTCATTAAAAAGGTAACCTGCACTAACGC
TGAGCTGGTGAAAGGAAGACAGTACTTAATTATGGGGAAAGAAGCTCTCCAGATAAAATACAATTTCACT
TTCAGGTACATCTACCCTTTAGATTCCTTGACCTGGATTGAATACTGGCCTAGAGACACAACATGTTCAT
CGTGTCAAGCATTTTTAGCTAATTTAGATGAATTTGCTGAAGACATCTTTTTAAATGGATGCTAAAATTC
CTGAAGTTCAGCTGCATACAGTTTGCACTTATGGACTCCTGTTGTTGAAGTTTGTTTTTTTTTCTCGTTT
TTTTGTCTTTAAACATTCACAGCTGGTCTTATTTGTAAAGCTCACTTTACTTAGAATTAGTGGCACTTGC
TTTTATTAGAGAATGATTTTAAACGCTGTAACTTTCTGAAATAACATGGCCTTGGAGGGCATGAAGACAG
ATACTCCTCCAAGGTTATTGGACACCGGAAACAATAAATTAGAACACCTCCTCAAACCTACCACTTAGGA
ATGTTTGCTGGAGCCGAAAGAACAGTCCATTGAAATGGAGTATTACAAAAACATGGCCTTTGCTTGAAAG
AAAATACCAGGGGACAGGAAACTGATCATTAAAGCCTGAGTTTGCTTTCAAACTGTGCTAAAAA
>gi|291575171|ref|NM_010406.2| Mus musculus hemolytic complement (Hc), mRNA
SEQ ID NO: 3
TTTAAAAGGAAAGTGGTTACAGGGAGGCCATGCCCATGGGTTTATGCCGCTACCAGCCATGGGTCTTTGG
GGAATACTTTGTCTTTTAATTTTCCTGGACAAAACTTGGGGACAGGAACAAACCTACGTCATTTCAGCAC
CCAAAATCCTCCGGGTCGGCTCGTCTGAAAATGTGGTAATTCAAGTCCATGGCTACACTGAAGCATTTGA
TGCAACTCTTTCTCTAAAAAGCTATCCTGACAAAAAAGTCACCTTCTCTTCAGGCTATGTTAATTTGTCC
CCGGAAAACAAATTCCAAAACGCGGCACTGTTGACACTACAGCCCAATCAAGTTCCTAGAGAAGAAAGCC
CAGTCTCTCACGTGTATCTGGAAGTTGTGTCAAAACACTTTTCAAAATCAAAGAAAATACCAATTACCTA
TAACAATGGAATTCTCTTCATCCATACAGACAAACCTGTTTACACGCCGGACCAGTCAGTAAAGATCAGA
GTCTATTCTCTGGGTGACGACTTGAAGCCAGCCAAACGGGAGACTGTCTTAACTTTCATAGACCCCGAAG
GATCAGAAGTTGACATTGTAGAAGAAAATGATTACACCGGAATTATCTCTTTTCCTGACTTCAAGATTCC
ATCTAATCCCAAGTATGGTGTTTGGACAATTAAAGCTAACTATAAGAAGGATTTTACAACAACTGGAACT
GCATACTTTGAAATTAAAGAATATGTCTTGCCACGATTCTCTGTTTCAATAGAACTAGAAAGAACCTTCA
TTGGCTATAAAAACTTTAAGAACTTTGAAATCACTGTGAAAGCAAGATATTTTTATAATAAAGTGGTACC
TGATGCTGAAGTGTATGCCTTTTTTGGATTGAGAGAGGACATAAAAGATGAGGAGAAGCAGATGATGCAC
AAAGCCACACAAGCCGCAAAGTTGGTTGACGGAGTTGCTCAGATCTCTTTTGATTCTGAAACAGCAGTTA
AAGAGCTGTCCTACAACAGTCTAGAAGACTTAAACAACAAGTACCTTTATATTGCAGTAACAGTCACAGA
ATCTTCAGGTGGATTTTCAGAAGAGGCAGAAATCCCTGGAGTCAAATATGTCCTCTCTCCCTACACACTG
AATTTGGTCGCTACTCCTCTTTTCGTGAAGCCCGGGATTCCATTTTCCATCAAGGCACAGGTTAAAGATT
CACTCGAGCAGGCGGTAGGAGGGGTCCCAGTAACTCTGATGGCACAAACAGTCGATGTGAATCAAGAGAC
ATCTGACTTGGAAACAAAGAGGAGCATCACTCATGACACTGATGGAGTAGCTGTGTTTGTGCTGAACCTC
CCATCAAATGTGACGGTGCTAAAGTTTGAGATCAGAACTGATGACCCAGAACTTCCCGAAGAAAATCAAG
CCAGCAAAGAGTACGAAGCAGTTGCGTACTCGTCTCTCAGCCAAAGTTACATTTACATCGCTTGGACTGA
AAACTACAAGCCCATGCTTGTGGGAGAATACCTGAATATTATGGTTACCCCCAAGAGCCCATATATCGAC
AAAATAACTCACTATAATTACTTGATTTTATCCAAAGGCAAAATTGTACAGTACGGCACAAGAGAGAAAC
TTTTCTCCTCAACTTATCAAAATATAAATATTCCAGTGACACAGAACATGGTTCCTTCAGCACGACTCCT
GGTCTATTACATAGTCACAGGGGAGCAAACAGCAGAATTAGTGGCTGACGCAGTCTGGATAAATATTGAG
GAGAAGTGTGGCAACCAGCTCCAGGTCCATCTGTCTCCAGATGAATATGTGTATTCTCCAGGCCAAACTG
TGTCCCTTGACATGGTGACTGAAGCAGACTCATGGGTAGCACTATCAGCAGTGGACAGAGCTGTGTATAA
AGTCCAGGGAAACGCCAAAAGGGCCATGCAAAGAGTCTTTCAAGCTTTGGATGAAAAGAGTGACCTGGGC
TGTGGGGCAGGTGGTGGCCATGACAATGCAGATGTATTCCATCTAGCTGGGCTCACCTTCCTCACCAACG
CAAACGCAGATGACTCCCATTATCGTGATGACTCTTGTAAAGAAATTCTCAGGTCAAAGAGAAACCTGCA
TCTCCTAAGGCAGAAAATAGAAGAACAAGCTGCTAAGTACAAACATAGTGTGCCAAAGAAATGCTGCTAT
GACGGAGCCCGAGTGAACTTCTACGAAACCTGTGAGGAGCGAGTGGCCCGGGTTACCATAGGCCCTCTCT
GCATCAGGGCCTTCAACGAGTGCTGTACTATTGCGAACAAGATCCGAkAAGAAAGCCCCCATAAACCTGT
CCAACTGGGAAGGATCCACATTAAGACCCTGTTACCAGTGATGAAGGCAGATATCCGAAGCTACTTTCCA
GAGAGCTGGCTATGGGAAATTCATCGCGTTCCCAAAAGAAAACAGCTGCAGGTCACGCTGCCTGACTCAC
TAACGACTTGGGAAATTCAAGGCATTGGCATTTCAGACAATGGTATATGTGTTGCTGATACACTCAAGGC
AAAGGTGTTCAAAGAAGTCTTCCTGGAGATGAACATACCATATTCTGTTGTGCGAGGAGAACAGATCCAA
TTGAAAGGAACTGTTTACAACTATATGACCTCAGGGACAAAGTTCTGTGTTAAAATGTCTGCTGTGGAGG
GGATCTGCACTTCAGGAAGCTCAGCTGCTAGCCTTCACACCTCCAGGCCCTCCAGATGTGTGTTCCAGAG
GATAGAGGGCTCGTCCAGTCACTTGGTGACCTTCACCCTGCTTCCTCTGGAAATTGGCCTTCACTCCATA
AACTTCTCACTAGAGACCTCATTTGGGAAAGACATCTTAGTAAAGACATTACGGGTAGTGCCAGAAGGAG
TCAAGAGGGAAAGCTATGCCGGCGTGATTCTGGACCCTAAGGGAATTCGTGGTATTGTTAACAGACGAAA
GGAATTCCCATACAGGATCCCATTAGATTTGGTCCCCAAGACCAAAGTTGAAAGGATTTTGAGTGTCAAA
GGACTGCTTGTAGGGGAGTTCTTGTCCACGGTTCTGAGTAAGGAAGGCATCAACATCCTAACCCACCTCC
CCAAGGGCAGTGCAGAGGCAGAGCTCATGAGCATAGCTCCGGTGTTCTATGTTTTCCACTACCTGGAAGC
AGGAAACCATTGGAATATTTTCTATCCTGATACACTGAGTAAAAGACAGAGCCTGGAGAAAAAAATAAAA
CAAGGGGTGGTGAGCGTCATGTCCTACAGAAACGCTGACTATTCCTACAGCATGTGGAAGGGGGCGAGCG
CTAGTACCTGGCTGACAGCTTTTGCTCTGAGAGTGCTTGGACAGGTGGCCAAGTATGTAAAACAGGATGA
AAACTCAATTTGTAACTCTTTGCTATGGCTGGTTGAGAAGTGTCAGCTGGAAAACGGCTCTTTCAAGGAA
AATTCCCAATATCTACCAATAAAATTACAGGGTACTTTGCCTGCTGAAGCCCAAGAGAAAACTTTGTATC
TTACAGCCTTTTCTGTGATTGGAATTAGAAAGGCAGTTGACATATGCCCCACCATGAAAATCCACACAGC
GCTAGATAAAGCCGACTCCTTCCTGCTTGAAAACACCCTGCCATCCAAGAGCACCTTCACACTGGCCATT
GTAGCCTATGCTCTTTCCCTAGGAGACAGAACCCACCCGAGGTTTCGTCTAATTGTGTCGGCCCTGAGGA
AGGAAGCTTTTGTTAAAGGTGATCCGCCCATTTACCGTTACTGGAGAGATACCCTCAAACGTCCAGACAG
CTCTGTGCCCAGCAGCGGCACAGCAGGTATGGTTGAAACCACAGCCTATGCTTTGCTCGCCAGCCTGAAA
CTGAAGGATATGAATTACGCCAACCCCATCATCAAGTGGCTATCTGAAGAGCAGAGGTATGGAGGCGGCT
TTTATTCCACCCAGGATACGATTAATGCCATCGAGGGCCTGACAGAATATTCACTCCTGTTAAAACAAAT
TCATTTGGATATGGACATCAATGTCGCCTACAAACACGAAGGTGACTTCCACAAGTATAAGGTGACAGAG
AAGCATTTCCTGGGGAGGCCAGTGGAGGTATCTCTCAATGATGACCTTGTTGTCAGCACAGGCTACAGCA
GTGGCTTGGCCACAGTATATGTAAAAACTGTGGTTCACAAAATTAGTGTCTCTGAGGAATTTTGCAGCTT
TTACTTGAAAATTGATACCCAAGATATTGAAGCATCCAGCCACTTCAGGCTCAGTGACTCTGGATTCAAG
CGCATAATAGCATGTGCCAGCTACAAGCCCAGCAAGGAGGAGTCAACATCCGGGTCCTCCCATGCAGTAA
TGGATATATCACTGCCGACTGGAATCGGAGCAAACGAGGAAGATTTACGGGCTCTTGTGGAAGGAGTGGA
TCAACTACTAACTGATTACCAGATCAAAGATGGCCATGTCATTCTGCAACTGAATTCGATCCCCTCCAGA
GATTTCCTCTGTGTCCGGTTCCGGATATTTGAACTTTTCCAAGTTGGGTTTCTGAATCCTGCTACCTTCA
CGGTGTACGAGTATCACAGACCAGATAAGCAGTGCACCATGATTTATAGCATTTCTGACACCAGGCTTCA
GAAAGTCTGTGAAGGAGCAGCTTGCACATGTGTGGAAGCTGACTGTGCGCAACTGCAGGCAGAAGTAGAC
CTAGCCATCTCTGCAGACTCCAGAAAAGAGAAAGCCTGTAAACCAGAGACTGCATATGCTTATAAAGTCA
GGATCACATCAGCCACTGAAGAAAATGTTTTTGTCAAGTACACTGCGACTCTTCTGGTCACTTACAAAAC
AGGGGAAGCTGCTGATGAGAATTCGGAGGTCACCTTCATTAAAAAGATGAGCTGTACCAATGCCAACCTG
GTGAAAGGGAAGCAGTATTTAATCATGGGCAAAGAGGTTCTGCAGATCAAACACAATTTCAGTTTCAAGT
ATATATACCCTCTAGATTCCTCCACCTGGATTGAATATTGGCCCACAGACACAACGTGTCCATCCTGTCA
AGCATTTGTAGAGAATTTGAATAACTTTGCTGAAGACCTCTTTTTAAACAGCTGTGAATGAkAAGTTCTG
CTGCACGAAGATTCCTCCTGCGGCGGGGGGATTGCTCCTCCTCTGGCTTGGAAACCTAGCCTAGAATCAG
ATACACTTTCTTTAGAGTAAAGCACAAGCTGATGAGTTACGACTTTGTGAAATGGATAGCCTTGAGGGGA
GGCGAAAACAGGTCCCCCAAGGCTATCAGATGTCAGTGCCAATAGACTGAAACAAGTCTGTAAAGTTAGC
AGTCAGGGGTGTTGGTTGGGGCCGGAAGAAGAGACCCACTGAAACTGTAGCCCCTTATCAAAACATATCC
TTGCTTGAAAGAAAAATACCAAGGACAGAAAATGCCATAAAATCTTGACTTTGCACTC
>gi|392346248|ref|XM_345342.4| PREDICTED: Rattus norvegicus complement component 5
(C5), mRNA
SEQ ID NO: 4
ATGGATAGCACAGAGACCGACAGATGTCCTACAGCCCGCCATCATCTTTCCGGAAACATTAACTCAGTGC
TTGCTGCCCTTGTAGGTGGGTTTTCGGAAGAGGCAGAAATTCCTGGCATCAAATACGTCCTCTCTCCCTA
TACACTGAATTTGGTCGCTACCCCTCTTTTCCTGAAGCCTGGGATTCCATTTTCCATCAAGGTACAGGTT
AAGGATTCACTCGAGCAGTTGGTAGGAGGGGTCCCAGTAACTCTGATGGCACAAACAGTCAATGTGAATC
AAGAGACATCTGACTTGGAACCAAAGAGGAGCATCACACACTCTGCTGATGGAGTGGCTTCATTTGTGGT
GAACCTCCCATCAGAAGTGACATCACTGAAGTTTGAGGTCAAAACTGATGCCCCGGAACTTCCCGAAGAA
AATCAAGCCAGCAAAGAATATGAAGCAGTTACATACTCATCCCTCAGCCAGAGTTACATTTACATTGGCT
GGACTGAAAACTACAAGCCCATGCTTGTGGGAGAATATCTGAATATTATCGTCACCCCCAAGAGTCCATA
TATTGACAAAATAACTCACTATAATTACTTGATTTTATCCAAAGGCAAAATTGTACAGTATGGCACAAAG
GAGAAACTTCTCTATTCATCTTATCAAAATATAAACATCCCAGTGACACAGGACATGGTTCCTTCAGCGC
GGCTCCTGGTCTATTACATAGTCACGGGGGAGCAGACAGCAGAATTGGTGGCTGACGCAGTCTGGATAAA
CATTGAGGAGAAGTGTGGCAACCAGCTCCAGGTCCATCTGTCTCCAGATAAAGACGTGTATTCTCCAGGC
CAAACTGTGTCCCTTGACATGGTGACTGAAGCAGACTCATGGGTGGCACTATCTGCGGTGGACAGCGCTG
TGTATGGAGTCCGGGGAAAAGCCAAAAGGGCCATGCAAAGAGTGTTCCAAGCTTTTGATGACAAGAGTGA
CCTGGGCTGTGGGGCAGGTGGTGGCCGTGACAATGTAGATGTATTCCATCTAGCTGGGCTCACCTTCCTC
ACCAATGCAAACGCAGATGACTCCCAATACCACGATGACTCTTGTAAGGAAATTCTCAGGCCAAAGAGAG
ACCTGCAGCTCCTGCATCAGAAAGTGGAAGAACAAGCTGCTAAATACAAACACCGTGTGCCCAAGAAATG
CTGTTATGATGGAGCCCGAGAAAACAAATACGAAACCTGTGAGCAGCGAGTTGCCCGGGTGACCATAGGC
CCACACTGCATCAGGGCCTTCAACGAGTGTTGTACTATTGCGGATAAGATCCGAAAAGAAAGCCACCACA
AAGGCATGCTGTTGGGAAGGATCCAAATAAAGGCCCTGTTACCAGTGATGAAGGCAGAAATCCGAAGCTA
CTTTCCAGAGAGCTGGCTATGGGAAGTTCATCGTGTTCCCAAAAGAAACCAGCTGCAGGTTGCACTGCCT
GACTCACTGACGACCTGGGAAATTCAAGGCATCGGCATCTCAGACAATGGTATATGTGTTGCTGACACAC
TCAAGGCAAAGGTGTTCAAAGATGTCTTCCTGGAGATGAACATACCATATTCTGTTGTACGAGGGGAGCA
GATCCAATTGAAGGGAACCGTTTACAATTATAGGACCTCTGGGACAATGTTCTGTGTTAAAATGTCTGCC
GTGGAGGGAATCTGCACTCCAGGAAGCTCGGCTGCTAGCCCTCAGACCTCTAGGTCCTCCAGATGTGTGC
GCCAGAGAATAGAGGGCTCCTCCAGTCACTTGGTGACCTTCAGCCTGCTTCCTCTGGAAATTGGCCTTCA
CTCCATAAACTTCTCACTAGAGACTTCATTTGGGAAAGAAATCTTAGTGAAGACATTACGGGTAGTGCCA
GAAGGGATCAAAAGGGAAAGCTATGCTGGTGTGACTCTGGACCCCAGGGGAGTTTATGGTATTGTTAACA
GACGAAAGGAATTCCCATACAGGATACCATTAGATTTGGTCCCCAAAACCAACGTCAAAAGGATTTTGAG
TGTAAAAGGACTGCTTATAGGGGAATTCTTGTCCACGGTTCTGAGTAAAGAAGGCATCGACATCCTAACC
CACCTCCCCAAGGGCAGCGCCGAGGCAGAACTCATGAGCATAGTCCCGGTGTTCTACGTTTTCCACTACC
TGGAAGCAGGAAACCATTGGAATATTTTCCACCCTGATACGTTAGCTAGAAAACAGAGCCTGCAGAAAAA
AATAAAAGAAGGGCTGGTGAGCGTCATGTCCTACAGAAACGCTGACTATTCCTACAGCATGTGGAAGGGA
GCAAGCTCTAGTGCCTGGCTGACAGCTTTTGCTCTGAGAGTGCTTGGACAGGTGAACAAGTATGTGAAAC
AAGACCAATACTCGATCTGTAACTCCTTGTTATGGCTGATTGAGAAGTGTCAGCTGGAAAACGGATCTTT
CAAGGAAAATTCCCAATATCTACCAATAAAATTACAGGGTACTTTGCCTGCTGAAGCCCAAGAGAACACT
TTATATCTTACAGCCTTTTCTGTGATTGGAATTAGAAAGGCTATTGGCATATGCCCCACGGAGAAAATCT
ACACAGCGCTGGCTAAAGCTGACTCCTTCCTACTTGAAAGGACCCTGCCTTCCAAGAGCACCTTCACCCT
GGCCATTGTGGCCTATGCTCTCTCCCTGGGAGACAGAACCCACCCGAAGTTTCGTTCTATTGTGTCAGCC
CTGAAGAGGGAAGCTTTGGTTAAAGGAGACCCGCCCATTTACCGTTTCTGGAGAGACACTCTCCAACGTC
CAGACAGCTCAGCACCCAACAGCGGCACAGCAGGTATGGTAGAAACCACGGCCTATGCTTTGCTCACCAG
CCTGAACCTGAAGGAGACGAGTTATGTCAACCCGATCATCAAGTGGCTATCTGAGGAGCAGAGGTATGGA
GGCGGCTTTTATTCCACCCAGGATACCATTAACGCCATCGAGGGCCTGACAGAGTATTCACTCCTGGTTA
AACAACTTCATTTGGATATGGATATCAATGTCTCCTACAAACACAAAGGGGATTTCTACCAGTATAAAGT
GACAGAGAAGAACTTCCTCGGGAGGCCAGTGGAGGTACCCCTCAATGATGACCTCATCGTCACCACAGGC
TATAGCAGTGGCTTGGCTACAGTATATGTAAAAACTGTGGTTCACAAAACTAGTGTCGCTGAGGAATTTT
GCAGCTTTTACTTGAAAATTGATACCCAAGAAGTTGAAGCCTCCAGCTACCTCAGCTACAGTGACTCGGG
ACACAAGCGCATAATAGCCTGTGCCAGCTACAAGCCCAGCAAGGAGGAGTCAGCATCTGGGTCCTCCCAT
GCAGTAATGGATATACTGCTGCCGACCGGAATCGGAGCAAACCAAGAAGATTTACGAGCTCTTGTGGAAG
GAGTAGATCAACTCCTAACTGATTACCAGATCAAAGACAGTCATGTTATTCTGCAATTGAATTCGATTCC
CTCCAGAGATTTCCTTTGTGTTCGGTTCCGGATATTTGAACTTTTCCAAGTTGGGTTTCTGAATCCTGCT
ACGTTCACGGTGTACGAGTATCACAGACCAGATAAGCAGTGTACCATGATTTACAGCACTTCTGACACCA
ACCTTCAGAGAGTCTGTGAAGGAGCGGCATGCAAATGCGTTGAAGCTGATTGTGGGCAACTGCAGGCAGA
ACTGGACCTGGCCATCTCTGCAGACACCAGGAAAGAAACAGCATGTAAACCAGAGATTGCATATGCTTAT
AAGGTCAGGATCACGTCGGCCACGGAAGAAAACATTTTTGTCAAGTACACTGCGACGCTTCTGGATATTT
ACAAAACAGGGGAAGCCGCTGCTGAGAAGGACTCTGAGATCACCTTCATTAAAAAGATAAGCTGTACCAA
CGCCAACCTGGTGAAAGGAAAGCAATATTTAATCATGGGCAAAGAGGCTCTGCAGATCAAACACAATTTC
AGTTTCAAGTATATATACCCTCTAGATTCCTCCACCTGGATTGAATATTGGCCCACAGACACAACGTGTC
CATCCTGCCAAGCGTTTGTAGCTAATTTGGACGAGTTCGCTGAAGACATCTTTCTAAATGGCTGTGAAAA
TGCCTGAGGAAGTTCTGCTGCGTGGCCTTCCCGGGTACTCCTGTTGGTGGCTCCTAGGAGCCAGGATCGC
TTGGAAACTTAGCCTAGAATCGGATACATTTTCTTTATAGTAAAGCGTAAGTTGAAGAGTTACTTTGTGA
AACAAAATAGCCTTGTGGAGAGCCGAAGGCAGGTCCCCCAAGGCTATTGGACATCAGCACCAATAAGCTG
GAACAAGTCTGTAACGTTAGCAGCCAGGGGTGTTTGTTGGGGCCGGAAGAAGAGACTCACTGAAATTGTA
GCCCCTTAGGAAAACATGGTCTTGCTTGAAAAAAAAAATACCAAGGACAGAAAATGCCATAAAAGCTTGA
CTTTGCACTCAACTGTA
Reverse Complement of SEQ ID NO: 1
SEQ ID NO: 5
TTTTTTTTTTTTTTTTTTGAAAGCAAACTCAGGCTTTAATGATCAGTTTCCTGTTCCTTGGTATTTTCTTTCAAGCAAAGGCC
ATGTTTTTGTAATACTCCCTTTCAATGGACTGTTCTTTCGGCCCCAGCAAACATTCCTGAGTGGTAGGTTTGAGGAGGTGTTC
CAATTTATTGTTTCCGGTGTCCAATAACCTTGGAGGAGTATCTGTCTTCATGCCCTCCAAGGCCATGTTATTTCAGAAAGTTA
CAGCATTTGAAATCATTCTCTAATAAAAGCAAGTGCCACTAATTCTAAGTAAAGTGAGCTTTACAAATAAGACCAGCTATGAA
TGTTTAAAAAAAGAAGAAAACAAAAAAACGAACTTCAACAACAGGAGTCCATAAGTGCAAACTGTATGCAGCTGAACTTCAGG
AATTTTAGCATCCATTTAAAAAGATATCTTCGGCAAATTCATCTAAATTAGCTAAAAATGCTTGACACGATGAACATGTTGTG
TCTCTAGGCCAGTATTCAATCCAGGTCAAGGAATCTAAAGGGTAGATGTACCTGAAACTGAAATTGTATTTTATCTGGAGGGC
TTCTTTACCCATAATTAAGTACTGTCTTCCTTTTACCAGCTCAGCGTTAGTACAGGTTACCTTTTTAATGAAGGTAATCTCAG
AGTCTTTCTCAGCAACAGCTTCCCCAGTTTTGTAGATATCCAGAAGGGTTGCCTTGTACTTGACAAAAACATTTTCTACAGTG
ATGGATGTGATGCTAACTTTATAAGCATATGCAATCTCTGGTTTACATGCTGTTTGTTTTCTTGTCTCTGCAGAGATTGTCAG
ATCCAATTCTTCCTGCATTTGCCCACAATCAGCTTCTACACACTTGCACGCGGCTCCTTCACAGACTTTCTGAATTTTGATAT
TGGAAGTGCTATAAAACATGGTACACTGTTTATCTGGTCTGTGGTATTCGTACACTGTGAAAGTGGCAGGACTGAGAAACCCA
ACTTCAAAGAGTTCAAATATCCGGAATCGTACACAAAGGAAATCACTGGAGGGAATCGAATTCAGTTGCAGAATAACATGTCC
ATCTTTGATTTGGTAATCAGTGAATAGTTGATCCACCCCTTCCACAAGGGCTTTTAAGTCTTCTTCATTTGCACTGATTCCAG
TAGGCAAGGAGATGTCCATCACCGCATGAGAGGATCCAGATGATGATTCTTCCCTGCTGGGCTTGTAGCTGGCACATGCTACT
ATGCGTTTGTAATCAGAGTTTCCGTAGCCTCTGTAGTGGGATGCTTCAATATCCTGAGTATCGATTTTCAAATAAAAGCTGCA
AACTTCCTCAGAGGTACTGGTTTTGTGAACTACAGTTGTTACATGTACTGTAGCCAAGCCACTGCCAAATCCTGTACTGACAA
TGAGGTCATCATTGAGAAGCACCTCTACTGGCCTCCCAAGGAAATTCTTGTCTGTCATTTTATAATTATGTAAGGCACCTTTA
TGCTTGTAAGAAACATCGATGTCCATACTCAAGCGGAGTTGTTTAACCAGGAGTGAATATTCCGTCAGGCCCTCAATGGCATT
GATTGTGTCCTGGGTTGAATAAAAGCCACCTCCATACCTCTGCTCTTCTGATAGCCATTTGATGACTGGGTTAACATAATTTA
TATCTTTCAAGTTCAGACTGGTGAGTAAAGCATAGGCAGTTGTTTCTACCATACGTGCCGTACCAGTGTTAGGTACAGAGCTG
TCTTTATGCTGAAGATTGTCTTTCCAAAAACGATAAATGGGTGGATTACCTTTAACCAAAGCTTCTCTCTTCAAAGCTGAAAC
AATTGAACGAAACTGTGGGTGAGTTTTATCTCCCAGGGAAAGAGCATACGCAGAAATGGCCAATGTAAAGGTGCTCTGGGCTG
GCAGTGTATTTTCAAGCAGAAAGTTGTCAGCTTTAATTAGAGCTGTGTCGATTTTCACCAGGGGGCATATATCGAAAGCCTTT
CTAATTCCAATCACAGTAAAGGCTGTAAGATATAAGCTGTTCTCTCGGGCTTCAACAGGCAAGGTACCCTGTAATTTTATTGG
TTGATACTGTGAATTTTCCTTGAAAGATCCATTATCTAATTGATAATTCTCAACTAGCCACAATAAAGAATTACAAATTGAAT
TTTGGTTCTGCTCTACGTATTTATTTACTTGTCCAAGTACTCTTAAAGCAAAAGCTGTTAACCAAGTGCTAGCACTTCCACCC
TTCCACACACTGTAAGAGTAGTCAGCATTTCTGTAGGACATAATGCTCAACATCCCTTCTTTTAATTTTTTCTTCAGTTTCTG
CTTTTCAATTAATGGGTCAGAATGAAAAATGTTCCAATGATTTCCTGTTTCCAGGTAGTGAAAAACATAGAATACTGGGACAA
CGCTCATCAGCTCCGCCTCTGCACTCCCTTTGGGGAGGTGGGTTAGGATATTGATGCCTTCCTGACTTAGAACTGCAGACAAG
ATCTCACCTACAAGCAGTCCTTTTACACTCAAAATCCTTTTGATTTCTGTTTTGGGGACCAAATCTAAGGGTATCCTGTATGG
GAACTCCTTTCGTCTGCTAATGGTACCATAAATACCCCTAGGATCCAAAGTAACACCAGAATAGCTTTCCCTTTTGACACCTT
CTGGCACCACTCGTAATGTTTTTACTAAGATTTCTTTTCCAAACCAAGTCTCCAGTGAAAAATTGATGTTGTGAAGGCCAATT
TCCAGAGGAAGCACAGTGAATGTCACCAAGTGACTGGAGGAGCCCTCTACTTTCTGGCGCACACATTTGGAGGACTTTGTGCC
CTGATGATCAATGACTGGGCTTTCCGAAGTGCAGATTCCCTCCACAGCAGACATTTTAACACAGAACTGCATCCCAGAAGTCC
TATAGTTGTAAACAGTTCCTTTCAATTGGATCTGTTCTCCTCGTACAACAGAATATGGTATATTCATTTCCAGGAAGACATCT
TTGAACACCTTTGCCTTGACAGTATCAGCAACACATATACCAGTGTTTGAAATGCCAACGCCTTGAATTTCCCAGGTGGTTAG
AGAATCAGGTAGGGCAAACTGCAACTGTTTTCTTCTGGGAACAAGATGAACTTCCCACAACCAGCTTTCTGGAAAATAACTCC
GAATTTCTGGCTTGCTTACTGGTAACAGGGTCTTCATGTGTAGCCTTCCCAATTGCATGTCTTTATGAGAGATATTAGCACGG
AGCTGGCTTGCGACGACACAACATTCAGTGAAAGCTTTGATGCATCTTGGCCCTAAACTAATCCGTGCAGCTCGCTGCTCACA
GGTTTCATCATTATTAACGCAGGCTCCATCGTAACAACATTTCTTCACTACTGAATGTTTATATTTAGCAGCTATTTCTTCTA
TCTTCTTTTGCAGCGTTCTTCTTGGCCTGAGAATTTCTTTACAAGGTTCATCATTTTCTTGGGAGTCATCTGCATTTGCATTA
GTGAGGAAGGTAAGTCCAGCTAGGTGGAACACATTGGCATTGTTGAGGCCACCACCTGCCCCACAGCCCAGATCACTCTTCTC
TAAGAATTGAAATACTCTTTCCAAGGGCTTTTTGGCTCCTCTTTGGACTCCATACACAGCACTGTCCACTGCTGCTAATGCCA
CCCAGGAATCCATTCCAGTTGCCATATTAAGAGACACAGTTTGGCCTGGAGAATATGCATCTGCATCAGGAGACAGATGAACC
TGGAGCTGGTTGCCACATTTTTCTTCAATATTTAACCAGACTGAATCAGACACTAATTCTGCTGTCTGTTCTCCTGTGACGAT
GTAATAGACCAGAAGTCGGGATGAAGGAACCATGTTCTGTGTTACTGGAATGTTTATACTTTGATAAGATGCATCTGAAAATT
TCTCCCTCGTGCCAAAGTGGATAATTTTGCCCTTGGATAAAATCAAGTAATTATAGTGAGTTATTTTGTCAATATATGGGCTT
TTGGGGGTAACAATAATATTCAGATGTTCTCCCACTAGCAAAGCCTTATGGTTATCAGTCCAATCAATATAAAGGTAACTTTG
GCTGAGAGATGAGTATGCTATTGCTCGGTAACCTTCCCTGGCCTGATTTTCTTCTGGAAGATCTGGAGCATCAGTTTTGACAT
TAAACTCCAGCACCGTCACTCCAGATGGGAGATTAAGCACAAAGGAAGCTACTCCATCATCAACACGTGTTACACTTTTGCTT
GGATCCAAGTCAGATGTCTCTTGGTTTACATCAATTGTTTGTGCATTCAGTGTTACTGGGACTCCTCCTACCAACTGGTCAAG
CGAATCTTTAACCTGCACCTTGATGGGATATGGAATCCCAGGCTTCAGGAAAAGAGGAGTAGCAACCAAATTCAGTTTGTAGG
GAGAGAGGACATATTTGATGCCAGGTATTTCTGCCTCTTCAGAAAATCCACCTGTAGACTCTATGACTGTTACAGCAATATAA
AGGTACTTGTTGTTTAAATCTTCTAAACTGTAGTATGACAGTTCTTTGACTGCTGTTTCAGAATCAAATGTGACTTGAGCAAT
TCCATTTATCAACATTGTGTTTTGCATTGCTGTTTGCATCATTTCTTTTTGATCATCTTTTAAGTCTTCTCTTATTCCAAATG
TGATATAAACGTCAGCCTCAGTGACTACTTTATTATAAAAATATCTTGCTTTTATAGTAATTTCAAAATTCTTAAAGTTCTTG
TAACCAATGAAATTATATTCTGGCTCGATTGAGACAGAAAAATGTGGCAAGACATATTCTTTAACTTCAAAATATGCGGTTCC
AGTTGTTGAAAAGTCCTCTTTATATTTAGCCTTGATCGTCCACATACCATATCTAGGATTAGACGGAATCTTGAAGTCAGGAA
AAGAGATAATTCCAATATGATCAATTTCTTCTACCATGTCAACTTCTGATCCTTCAGGATCTATGAAAGTTAAGACAGTTTCT
CTTTTGGCTGGCTTCAAGTCGTCATTCAACGAATAAACTCTAACTTTTACTGACTGGTCTGGAGTATAAACAGGTTTGTCTGT
ATGAATGAAGAGAAATCCATTGTCATAGGTTATTGGCATTCTTTTTGATTTTGAAAAATGCTTTGATACAACTTCCAAATACA
CATAAGAAACTGGGTTTTGTCCTCCAGGCAATTGTTTTGGTTGTATTGTTAAGATTGCAGAGTTTTGGAATTTATTCTCTGAG
GATAAATGAACATGGCCTGAGGAGTAACTAAATTTTTTATCAGGATAACTTTTAATAGAGATTGTTGCATCAAATGCTTCAGT
GTATCCATAAACTTGAATCACAATATTTTCAGATGCTCCAACACGGAATATTTTTGGTGCTGAAATGACATATGTTTGCTCCT
GTCCCCAGGTTTTCCCCAGGAAGATTAAAAAACAAAGTATTCCCAAAAGGCCCATGGTTGGAGGTAGCAGGAAACCACGGATA
TA
Reverse Complement of SEQ ID NO: 2
SEQ ID NO: 6
TTTTTAGCACAGTTTGAAAGCAAACTCAGGCTTTAATGATCAGTTTCCTGTCCCCTGGTATTTTCTTTCAAGCAAAGGCCATG
TTTTTGTAATACTCCATTTCAATGGACTGTTCTTTCGGCTCCAGCAAACATTCCTAAGTGGTAGGTTTGAGGAGGTGTTCTAA
TTTATTGTTTCCGGTGTCCAATAACCTTGGAGGAGTATCTGTCTTCATGCCCTCCAAGGCCATGTTATTTCAGAAAGTTACAG
CGTTTAAAATCATTCTCTAATAAAAGCAAGTGCCACTAATTCTAAGTAAAGTGAGCTTTACAAATAAGACCAGCTGTGAATGT
TTAAAGACAAAAAAACGAGAAAAAAAAACAAACTTCAACAACAGGAGTCCATAAGTGCAAACTGTATGCAGCTGAACTTCAGG
AATTTTAGCATCCATTTAAAAAGATGTCTTCAGCAAATTCATCTAAATTAGCTAAAAATGCTTGACACGATGAACATGTTGTG
TCTCTAGGCCAGTATTCAATCCAGGTCAAGGAATCTAAAGGGTAGATGTACCTGAAAGTGAAATTGTATTTTATCTGGAGAGC
TTCTTTCCCCATAATTAAGTACTGTCTTCCTTTCACCAGCTCAGCGTTAGTGCAGGTTACCTTTTTAATGAAGGTGATTTCAG
AGTCTTTTTCAGCAACAGCTTCCCCAGTTTTGTAGATATCCAGAAGGGTTGCCTTGTACTTGACAAAAACATTTTCTGTAGTG
ATGGATGTGATGATAACTTTATAAGCATATGCAATCTCTGGGTTACATGCTGTTTGTTTTCTAGTCTCTGCAGAGATTGTCAG
ATCCAATTCTTTCTGCATTTGCCCACAATCAGCTTCTATACACTTGCACGTGGCTCCTTCACAGACTTTCTGAATTTTGATAT
TGGAAGTGCTATAAAACATGGTACACTGTTTATCTGGTCTGTGGTATTCATACACTGTGAAAGTGGCAGGACTAAGAAACCCA
ACTTCAAAGAGTTCAAAAATCCGGAATCGTACACAAAGGAAATCACTGGAGGGGATCGAATTCAGTTGCAGAATAACATGTCC
ATCTTTTATTTGGTAATCAGTGAATAGCTGATCCACCCCTTCCACAAGAGCTTTTAAGTCTTCTTCATTTGCATTGATTCCAG
TAGGCAAGGAGATGTCCATCACTGCATGAGAGGATCCAGAAGATGATTCTTCCTTGCTGGGCTTGTAGCTGGCACATGCTACT
ATGCGTTTGTAATCAGAGTTTCCGTAGCCTCTGTAGTGGGATGCTTCAATATCCTGAGTATCAATTTTCAAATAAAAGCTGCA
AACTTCCTCAGAGGTACTGGTTTTGTGAACTACAGTTGTTACATGTACCGTAGCCAAGCCACTGCCAAATCCTGTACTGACAA
CGAGGTCATCATTGAGAAGCACCTCTACTGGCCTCCCAAGGAAATTCTTGTCTGTCATTTTATAATTATGTAAGGGACCTTTA
TGCTTGTAAGCAACATCGATGTCCATATTCAAGCGGAGCTGTTTAACCAGGAGTGAATATTCTGTCAGGCCCTCGATGGCATT
GATTGTGTCCTGGGTTGAATAAAAGCCACCTCCATACCTCTGCTCTTCTGATAGCCATTTGATGATTGGGTTAACATAATTTA
TGTCTTTCAAGTTCAGACTGGTGAGTAAAGCATAGGCAGTTGTTTCTACCATACGTGCTGTACCAGTGTTAGGTACAGAGCTG
TCTTTATGTTGAAGACTGTCTTTCCAAAAACGATAAATGGGTGGATTACCTTTAACCAAAGCTTCTCTCTTCAAAGCTGAAAC
AATTGAACAAAACTGTGGGTGAGTTTTATCTCCCAGGGAAAGAGCATAGGCAGAAATGGCCAATGTAAAGGTGCTCTGGGCTG
GCAGTGTATTTTCAAGCAGAAAGGTGTCAGCTTTAATTAGAGCTGTGTTGATTTTCTGTAATTTTATTGGTTGATACTGTGAA
TTTTCCTTGAAGGATCCATTATCTAACTGATAATTCTCAACCAGCCACAATAAAGAATTACATATTGAATTTTGGTTCTGCTC
TACATATTTATGTACTTGTCCAAGTACTCTTAAAGCAAAAGCTGTTAACCAAGTGCTAGCACTGCCACCCTTCCACACGCTGT
AAGAATAGTCAGCATTTCTGTAGGACATAATGCTCACCATCCCTTCTTTTAATTTTTTCTCCAGGTTCCGCTTTTCAATTAAT
GGGTCGGAATGAAAAATGTTCCAATGATTTCCTGTTTCCAGGTAGTGAAAAACATAGAATACTGGGACAACGCTCATCAGCTC
CGCCTCTGCACTCCCTTTGGGGAGGTGGGTTAGGATATTGATGCCTTCCCGACTTAGAACTGCAGACAAGATCTCACCTACAA
GCAGTCCTTTTACACTCAAAATCCTTTTGATTTCTGTTTTGGGGACCAAATCTAATGGTATCCTGTATGGGAACTCCTTTCGN
NNNNNNNNNNNNNNNNNNNCATAAATACCCCTAGGATCCAAAGTAATACCAGAATAGCTTTCCCTTTTGACACCTTCTGGCAC
CACTCGTAACGATTTTACTAAGATTTCTTTTCCAAACGAAGTCTCCAGTGAGAAATTGATGTTCTGAAGGCCAATTTCCAGAG
GAAGCACAGTAAAGGTCACCAAGTGATTAGAGGAGCCCTCTACTTTCTGTCGCACACATTTGGAGGACTTTGTGCCCTGATGA
TCAATGACTGGGCTTTCTGAAGTGCAGATTCCCTCCACAGCAGACATTTTAACACAGAACTGCATCCCAGAAGTCCTATAGTT
GTAAACAGTTCCTTTCAACTGGACCTGTTCTCCTCGTACAACAGAATATGGTATATTCATTTCCAGGAAGACATCTTTGAACA
CCTTTGCCTTAATAGTATCAGCAACACATATACCACTGTTTGAAATGCCAACACCTTGAATTTCCCAGGTAGTTACAGAATCA
GGTAGGGCAAACTGCAACTGTTTTCTTCTGGGAACAAGATGAACTTCCCATAACCAGCTTTCTGGAAAATAACTCCGAATTTC
TGGCTTGCTTACTGGTAACAGGGTCTTCATGTGTAGCCTTCCCAATTGCAAGTCTTTATGAGAGTTATTAGCACGGAGCTGGC
TTGCGACGACACAACATTCAGTGAAAGCTTTGACGCATCTCGGCCCTACACTAATCCGTGCAGCTCGCTGCTCACAGGTTTCA
TCATGATTAATACGGACTCCATCGTAACAACATTTCTTCACTACTAAATGTTTATATTTAGCAGCTATTTCTTCTATCTTCTC
TTGTAGCATTCTTCTTGGCCTGATAATTTCTTTACAAGGTTCATCATTTTCTTGGGAGTCATCTGCATTTGCATTAGTGAGGA
AGGTAAGTCCAGCTAGGTGGAACACATTGGCATTGTTGAGGCCACCACCTGCCCCACAGCCCAGATCACTCTTCTCTAAGAAT
TGAAATACTCTTTCCAAGGGCTTTTTGGCTCTTCTTTGGACTCCATACACAGCGCTGTCCACTGCTGTTAATGCCACCCAGGA
ATCCATCCCAGTTACCATATTAAGAGACACAGTTTGGCCTGGAGAATATGTATCTGCATCAGGAGACAGATGAACCTGGAGCT
GGTTGCCACATTTTTCTTCAATATTTAACCAGACTGAATCAGACACTAATTCTGCTGTCTGCTCTCCTGTGACGATGTAATAG
ACCAGGAGTCGGGATGAAGGAACCATGTTCTGCGTTACTGGAATGTTTATACTTTGATAAGATGCATCTGAAAGTTTCTCCCT
TGTGCCAAAGTGGATAATTTTGCCCTTGGATAAAATCAAGTAATTATAGTGAGTTATTTTGTCAATATATGGGCTTTTGGGGG
TAACAATAATATTCAAATATTCTCCCACTAGCAAAGCCTTGTGGTTATCAGTCCAATCGATATAAAGGTAACTTTGGCTGAGA
GATGAGTATGCTATTGCTCGGTAACCTTCCCTGGCCTGATTTTCGTCTGGAAGATCTGGAGCATCAGTTTTGACATTAAACTC
CAGCACCGTCACTCCAGATGGGAGATTAACCACAAACGAAGCTACTCCATCATCAACACGTGTTACACTTTTCCTTGGCTCCA
AGTCAGATGTCTCTTGGTTGACATCAATTGTTTGTGCATTCAGTGTTACTGGGACCCCTCCTACCAACTGGTCAAGCGCATCT
TTAACCTGCACCTTGATGGAATATGGAATCCCAGGCTTCAGGAAAAGAGGAGTAGCAACCAAATTCAGTTTGTAGGGAGAGAG
GACATATTTGATGCCAGGTATTTCTGCCTCTTCAGAAAATCCACCTGTAGACTCTATGACTGTTACAGCAATATAAAGGTACT
TGTTGTTTAAATCTTCTAAACTGTAGTATGACAGTTCTTTGACTGCTGTTTCAGAATCAAATGTGACTTGAGCAATTCCATTT
ATCAACATTGTGTTTTGCATTGCTGTTTGCATCATTTCTTTTTGATCATCTTTTAAGTCTTCTCTTATTCCAAATGTGATATA
AACATCAGCCTCAGTGACTACTTTATTATAAAAATATCTTGCTTTTATAGTAATTTCAAAATTCTTAAAGTTCTTATAACCAA
TGAAATTACTTTCTGGTTCTACTGAGACAGAAAAATGTGGCAAGACATATTCTTTAACTTCAAAAAATGCAGTTCCAGTTGTT
GAAAAGTCCTCTTTATATTTAGCCTGGATCATCCACATACCATATCTAGGATTAGACGGAATCTTGAAGTCAGGAAAAGAGAT
AATTCCAATATGATCAATTTCTTCTACCATGTCAATTTCTGATCCTTCAGGATCTATGAAAGTTAAGACAGTTTCTCTTTTGG
CTGGCTTCAAGTCATCATTCAACGAATAAACTCTAACCTTTACTGATTGGTCTGGAGTATAAACAGGTTTGTCTGTATGAATG
AAGAGAAATCCATTGTCATAGGTTATTGGAATTTTTTTTGATTTTGAAAAATGCTTTGATACAACTTCCAAATACACATAAGA
AACTTGGTTTTGTCCTCCAGGTAATTGTTTTGGTTGTATTGTTAAGACTGCCGAGTTTTGGAATTTATTCTCTGAGGATAAAT
GAACATGGCCTGAGGAGTAACTAAATTTTTTATCAGGATAACTTTTAATAGAGATTGTTGCATCAAATGCTTCAGTGTATCCA
TAAACTTGAATCACAATGTTTTCAGATGCTCCAACACGGAATATTTTTGGTGCTGAAATGACATATGTTTGCTCCTGTCCCCA
AGTTTTTCCCAGGAAGATTAAAAAACAAAGTATTCCCAAAAGGCCCATGGTTGGAGGTAGCAGGAAATCATG
Reverse Complement of SEQ ID NO: 3
SEQ ID NO: 7
GAGTGCAAAGTCAAGATTTTATGGCATTTTCTGTCCTTGGTATTTTTCTTTCAAGCAAGGATATGTTTTGATAAGGGGCTACA
GTTTCAGTGGGTCTCTTCTTCCGGCCCCAACCAACACCCCTGACTGCTAACTTTACAGACTTGTTTCAGTCTATTGGCACTGA
CATCTGATAGCCTTGGGGGACCTGTTTTCGCCTCCCCTCAAGGCTATCCATTTCACAAAGTCGTAACTCATCAGCTTGTGCTT
TACTCTAAAGAAAGTGTATCTGATTCTAGGCTAGGTTTCCAAGCCAGAGGAGGAGCAATCCCCCCGCCGCAGGAGGAATCTTC
GTGCAGCAGAACTTTTCATTCACAGCTGTTTAAAAAGAGGTCTTCAGCAAAGTTATTCAAATTCTCTACAAATGCTTGACAGG
ATGGACACGTTGTGTCTGTGGGCCAATATTCAATCCAGGTGGAGGAATCTAGAGGGTATATATACTTGAAACTGAAATTGTGT
TTGATCTGCAGAACCTCTTTGCCCATGATTAAATACTGCTTCCCTTTCACCAGGTTGGCATTGGTACAGCTCATCTTTTTAAT
GAAGGTGACCTCCGAATTCTCATCAGCAGCTTCCCCTGTTTTGTAAGTGACCAGAAGAGTCGCAGTGTACTTGACAAAAACAT
TTTCTTCAGTGGCTGATGTGATCCTGACTTTATAAGCATATGCAGTCTCTGGTTTACAGGCTTTCTCTTTTCTGGAGTCTGCA
GAGATGGCTAGGTCTACTTCTGCCTGCAGTTGCGCACAGTCAGCTTCCACACATGTGCAAGCTGCTCCTTCACAGACTTTCTG
AAGCCTGGTGTCAGAAATGCTATAAATCATGGTGCACTGCTTATCTGGTCTGTGATACTCGTACACCGTGAAGGTAGCAGGAT
TCAGAAACCCAACTTGGAAAAGTTCAAATATCCGGAACCGGACACAGAGGAAATCTCTGGAGGGGATCGAATTCAGTTGCAGA
ATGACATGGCCATCTTTGATCTGGTAATCAGTTAGTAGTTGATCCACTCCTTCCACAAGAGCCCGTAAATCTTCCTCGTTTGC
TCCGATTCCAGTCGGCAGTGATATATCCATTACTGCATGGGAGGACCCGGATGTTGACTCCTCCTTGCTGGGCTTGTAGCTGG
CACATGCTATTATGCGCTTGAATCCAGAGTCACTGAGCCTGAAGTGGCTGGATGCTTCAATATCTTGGGTATCAATTTTCAAG
TAAAAGCTGCAAAATTCCTCAGAGACACTAATTTTGTGAACCACAGTTTTTACATATACTGTGGCCAAGCCACTGCTGTAGCC
TGTGCTGACAACAAGGTCATCATTGAGAGATACCTCCACTGGCCTCCCCAGGAAATGCTTCTCTGTCACCTTATACTTGTGGA
AGTCACCTTCGTGTTTGTAGGCGACATTGATGTCCATATCCAAATGAATTTGTTTTAACAGGAGTGAATATTCTGTCAGGCCC
TCGATGGCATTAATCGTATCCTGGGTGGAATAAAAGCCGCCTCCATACCTCTGCTCTTCAGATAGCCACTTGATGATGGGGTT
GGCGTAATTCATATCCTTCAGTTTCAGGCTGGCGAGCAAAGCATAGGCTGTGGTTTCAACCATACCTGCTGTGCCGCTGCTGG
GCACAGAGCTGTCTGGACGTTTGAGGGTATCTCTCCAGTAACGGTAAATGGGCGGATCACCTTTAACAAAAGCTTCCTTCCTC
AGGGCCGACACAATTAGACGAAACCTCGGGTGGGTTCTGTCTCCTAGGGAAAGAGCATAGGCTACAATGGCCAGTGTGAAGGT
GCTCTTGGATGGCAGGGTGTTTTCAAGCAGGAAGGAGTCGGCTTTATCTAGCGCTGTGTGGATTTTCATGGTGGGGCATATGT
CAACTGCCTTTCTAATTCCAATCACAGAAAAGGCTGTAAGATACAAAGTTTTCTCTTGGGCTTCAGCAGGCAAAGTACCCTGT
AATTTTATTGGTAGATATTGGGAATTTTCCTTGAAAGAGCCGTTTTCCAGCTGACACTTCTCAACCAGCCATAGCAAAGAGTT
ACAAATTGAGTTTTCATCCTGTTTTACATACTTGGCCACCTGTCCAAGCACTCTCAGAGCAAAAGCTGTCAGCCAGGTACTAG
CGCTCGCCCCCTTCCACATGCTGTAGGAATAGTCAGCGTTTCTGTAGGACATGACGCTCACCACCCCTTGTTTTATTTTTTTC
TCCAGGCTCTGTCTTTTACTCAGTGTATCAGGATAGAAAATATTCCAATGGTTTCCTGCTTCCAGGTAGTGGAAAACATAGAA
CACCGGAGCTATGCTCATGAGCTCTGCCTCTGCACTGCCCTTGGGGAGGTGGGTTAGGATGTTGATGCCTTCCTTACTCAGAA
CCGTGGACAAGAACTCCCCTACAAGCAGTCCTTTGACACTCAAAATCCTTTCAACTTTGGTCTTGGGGACCAAATCTAATGGG
ATCCTGTATGGGAATTCCTTTCGTCTGTTAACAATACCACGAATTCCCTTAGGGTCCAGAATCACGCCGGCATAGCTTTCCCT
CTTGACTCCTTCTGGCACTACCCGTAATGTCTTTACTAAGATGTCTTTCCCAAATGAGGTCTCTAGTGAGAAGTTTATGGAGT
GAAGGCCAATTTCCAGAGGAAGCAGGGTGAAGGTCACCAAGTGACTGGACGAGCCCTCTATCCTCTGGAACACACATCTGGAG
GGCCTGGAGGTGTGAAGGCTAGCAGCTGAGCTTCCTGAAGTGCAGATCCCCTCCACAGCAGACATTTTAACACAGAACTTTGT
CCCTGAGGTCATATAGTTGTAAACAGTTCCTTTCAATTGGATCTGTTCTCCTCGCACAACAGAATATGGTATGTTCATCTCCA
GGAAGACTTCTTTGAACACCTTTGCCTTGAGTGTATCAGCAACACATATACCATTGTCTGAAATGCCAATGCCTTGAATTTCC
CAAGTCGTTAGTGAGTCAGGCAGCGTGACCTGCAGCTGTTTTCTTTTGGGAACGCGATGAATTTCCCATAGCCAGCTCTCTGG
AAAGTAGCTTCGGATATCTGCCTTCATCACTGGTAACAGGGTCTTAATGTGGATCCTTCCCAGTTGGACAGGTTTATGGGGGC
TTTCTTTTCGGATCTTGTTCGCAATAGTACAGCACTCGTTGAAGGCCCTGATGCAGAGAGGGCCTATGGTAACCCGGGCCACT
CGCTCCTCACAGGTTTCGTAGAAGTTCACTCGGGCTCCGTCATAGCAGCATTTCTTTGGCACACTATGTTTGTACTTAGCAGC
TTGTTCTTCTATTTTCTGCCTTAGGAGATGCAGGTTTCTCTTTGACCTGAGAATTTCTTTACAAGAGTCATCACGATAATGGG
AGTCATCTGCGTTTGCGTTGGTGAGGAAGGTGAGCCCAGCTAGATGGAATACATCTGCATTGTCATGGCCACCACCTGCCCCA
CAGCCCAGGTCACTCTTTTCATCCAAAGCTTGAAAGACTCTTTGCATGGCCCTTTTGGCGTTTCCCTGGACTTTATACACAGC
TCTGTCCACTGCTGATAGTGCTACCCATGAGTCTGCTTCAGTCACCATGTCAAGGGACACAGTTTGGCCTGGAGAATACACAT
ATTCATCTGGAGACAGATGGACCTGGAGCTGGTTGCCACACTTCTCCTCAATATTTATCCAGACTGCGTCAGCCACTAATTCT
GCTGTTTGCTCCCCTGTGACTATGTAATAGACCAGGAGTCGTGCTGAAGGAACCATGTTCTGTGTCACTGGAATATTTATATT
TTGATAAGTTGAGGAGAAAAGTTTCTCTCTTGTGCCGTACTGTACAATTTTGCCTTTGGATAAAATCAAGTAATTATAGTGAG
TTATTTTGTCGATATATGGGCTCTTGGGGGTAACCATAATATTCAGGTATTCTCCCACAAGCATGGGCTTGTAGTTTTCAGTC
CAAGCGATGTAAATGTAACTTTGGCTGAGAGACGAGTACGCAACTGCTTCGTACTCTTTGCTGGCTTGATTTTCTTCGGGAAG
TTCTGGGTCATCAGTTCTGATCTCAAACTTTAGCACCGTCACATTTGATGGGAGGTTCAGCACAAACACAGCTACTCCATCAG
TGTCATGAGTGATGCTCCTCTTTGTTTCCAAGTCAGATGTCTCTTGATTCACATCGACTGTTTGTGCCATCAGAGTTACTGGG
ACCCCTCCTACCGCCTGCTCGAGTGAATCTTTAACCTGTGCCTTGATGGAAAATGGAATCCCGGGCTTCACGAAAAGAGGAGT
AGCGACCAAATTCAGTGTGTAGGGAGAGAGGACATATTTGACTCCAGGGATTTCTGCCTCTTCTGAAAATCCACCTGAAGATT
CTGTGACTGTTACTGCAATATAAAGGTACTTGTTGTTTAAGTCTTCTAGACTGTTGTAGGACAGCTCTTTAACTGCTGTTTCA
GAATCAAAAGAGATCTGAGCAACTCCGTCAACCAACTTTGCGGCTTGTGTGGCTTTGTGCATCATCTGCTTCTCCTCATCTTT
TATGTCCTCTCTCAATCCAAAAAAGGCATACACTTCAGCATCAGGTACCACTTTATTATAAAAATATCTTGCTTTCACAGTGA
TTTCAAAGTTCTTAAAGTTTTTATAGCCAATGAAGGTTCTTTCTAGTTCTATTGAAACAGAGAATCGTGGCAAGACATATTCT
TTAATTTCAAAGTATGCAGTTCCAGTTGTTGTAAAATCCTTCTTATAGTTAGCTTTAATTGTCCAAACACCATACTTGGGATT
AGATGGAATCTTGAAGTCAGGAAAAGAGATAATTCCGGTGTAATCATTTTCTTCTACAATGTCAACTTCTGATCCTTCGGGGT
CTATGAAAGTTAAGACAGTCTCCCGTTTGGCTGGCTTCAAGTCGTCACCCAGAGAATAGACTCTGATCTTTACTGACTGGTCC
GGCGTGTAAACAGGTTTGTCTGTATGGATGAAGAGAATTCCATTGTTATAGGTAATTGGTATTTTCTTTGATTTTGAAAAGTG
TTTTGACACAACTTCCAGATACACGTGAGAGACTGGGCTTTCTTCTCTAGGAACTTGATTGGGCTGTAGTGTCAACAGTGCCG
CGTTTTGGAATTTGTTTTCCGGGGACAAATTAACATAGCCTGAAGAGAAGGTGACTTTTTTGTCAGGATAGCTTTTTAGAGAA
AGAGTTGCATCAAATGCTTCAGTGTAGCCATGGACTTGAATTACCACATTTTCAGACGAGCCGACCCGGAGGATTTTGGGTGC
TGAAATGACGTAGGTTTGTTCCTGTCCCCAAGTTTTGTCCAGGAAAATTAAAAGACAAAGTATTCCCCAAAGACCCATGGCTG
GTAGCGGCATAAACCCATGGGCATGGCCTCCCTGTAACCACTTTCCTTTTAAA
Reverse Complement of SEQ ID NO: 4
SEQ ID NO: 8
TACAGTTGAGTGCAAAGTCAAGCTTTTATGGCATTTTCTGTCCTTGGTATTTTTTTTTTCAAGCAAGACCATGTTTTCCTAAG
GGGCTACAATTTCAGTGAGTCTCTTCTTCCGGCCCCAACAAACACCCCTGGCTGCTAACGTTACAGACTTGTTCCAGCTTATT
GGTGCTGATGTCCAATAGCCTTGGGGGACCTGCCTTCGGCTCTCCACAAGGCTATTTTGTTTCACAAAGTAACTCTTCAACTT
ACGCTTTACTATAAAGAAAATGTATCCGATTCTAGGCTAAGTTTCCAAGCGATCCTGGCTCCTAGGAGCCACCAACAGGAGTA
CCCGGGAAGGCCACGCAGCAGAACTTCCTCAGGCATTTTCACAGCCATTTAGAAAGATGTCTTCAGCGAACTCGTCCAAATTA
GCTACAAACGCTTGGCAGGATGGACACGTTGTGTCTGTGGGCCAATATTCAATCCAGGTGGAGGAATCTAGAGGGTATATATA
CTTGAAACTGAAATTGTGTTTGATCTGCAGAGCCTCTTTGCCCATGATTAAATATTGCTTTCCTTTCACCAGGTTGGCGTTGG
TACAGCTTATCTTTTTAATGAAGGTGATCTCAGAGTCCTTCTCAGCAGCGGCTTCCCCTGTTTTGTAAATATCCAGAAGCGTC
GCAGTGTACTTGACAAAAATGTTTTCTTCCGTGGCCGACGTGATCCTGACCTTATAAGCATATGCAATCTCTGGTTTACATGC
TGTTTCTTTCCTGGTGTCTGCAGAGATGGCCAGGTCCAGTTCTGCCTGCAGTTGCCCACAATCAGCTTCAACGCATTTGCATG
CCGCTCCTTCACAGACTCTCTGAAGGTTGGTGTCAGAAGTGCTGTAAATCATGGTACACTGCTTATCTGGTCTGTGATACTCG
TACACCGTGAACGTAGCAGGATTCAGAAACCCAACTTGGAAAAGTTCAAATATCCGGAACCGAACACAAAGGAAATCTCTGGA
GGGAATCGAATTCAATTGCAGAATAACATGACTGTCTTTGATCTGGTAATCAGTTAGGAGTTGATCTACTCCTTCCACAAGAG
CTCGTAAATCTTCTTGGTTTGCTCCGATTCCGGTCGGCAGCAGTATATCCATTACTGCATGGGAGGACCCAGATGCTGACTCC
TCCTTGCTGGGCTTGTAGCTGGCACAGGCTATTATGCGCTTGTGTCCCGAGTCACTGTAGCTGAGGTAGCTGGAGGCTTCAAC
TTCTTGGGTATCAATTTTCAAGTAAAAGCTGCAAAATTCCTCAGCGACACTAGTTTTGTGAACCACAGTTTTTACATATACTG
TAGCCAAGCCACTGCTATAGCCTGTGGTGACGATGAGGTCATCATTGAGGGGTACCTCCACTGGCCTCCCGAGGAAGTTCTTC
TCTGTCACTTTATACTGGTAGAAATCCCCTTTGTGTTTGTAGGAGACATTGATATCCATATCCAAATGAAGTTGTTTAACCAG
GAGTGAATACTCTGTCAGGCCCTCGATGGCGTTAATGGTATCCTGGGTGGAATAAAAGCCGCCTCCATACCTCTGCTCCTCAG
ATAGCCACTTGATGATCGGGTTGACATAACTCGTCTCCTTCAGGTTCAGGCTGGTGAGCAAAGCATAGGCCGTGGTTTCTACC
ATACCTGCTGTGCCGCTGTTGGGTGCTGAGCTGTCTGGACGTTGGAGAGTGTCTCTCCAGAAACGGTAAATGGGCGGGTCTCC
TTTAACCAAAGCTTCCCTCTTCAGGGCTGACACAATAGAACGAAACTTCGGGTGGGTTCTGTCTCCCAGGGAGAGAGCATAGG
CCACAATGGCCAGGGTGAAGGTGCTCTTGGAAGGCAGGGTCCTTTCAAGTAGGAAGGAGTCAGCTTTAGCCAGCGCTGTGTAG
ATTTTCTCCGTGGGGCATATGCCAATAGCCTTTCTAATTCCAATCACAGAAAAGGCTGTAAGATATAAAGTGTTCTCTTGGGC
TTCAGCAGGCAAAGTACCCTGTAATTTTATTGGTAGATATTGGGAATTTTCCTTGAAAGATCCGTTTTCCAGCTGACACTTCT
CAATCAGCCATAACAAGGAGTTACAGATCGAGTATTGGTCTTGTTTCACATACTTGTTCACCTGTCCAAGCACTCTCAGAGCA
AAAGCTGTCAGCCAGGCACTAGAGCTTGCTCCCTTCCACATGCTGTAGGAATAGTCAGCGTTTCTGTAGGACATGACGCTCAC
CAGCCCTTCTTTTATTTTTTTCTGCAGGCTCTGTTTTCTAGCTAACGTATCAGGGTGGAAAATATTCCAATGGTTTCCTGCTT
CCAGGTAGTGGAAAACGTAGAACACCGGGACTATGCTCATGAGTTCTGCCTCGGCGCTGCCCTTGGGGAGGTGGGTTAGGATG
TCGATGCCTTCTTTACTCAGAACCGTGGACAAGAATTCCCCTATAAGCAGTCCTTTTACACTCAAAATCCTTTTGACGTTGGT
TTTGGGGACCAAATCTAATGGTATCCTGTATGGGAATTCCTTTCGTCTGTTAACAATACCATAAACTCCCCTGGGGTCCAGAG
TCACACCAGCATAGCTTTCCCTTTTGATCCCTTCTGGCACTACCCGTAATGTCTTCACTAAGATTTCTTTCCCAAATGAAGTC
TCTAGTGAGAAGTTTATGGAGTGAAGGCCAATTTCCAGAGGAAGCAGGCTGAAGGTCACCAAGTGACTGGAGGAGCCCTCTAT
TCTCTGGCGCACACATCTGGAGGACCTAGAGGTCTGAGGGCTAGCAGCCGAGCTTCCTGGAGTGCAGATTCCCTCCACGGCAG
ACATTTTAACACAGAACATTGTCCCAGAGGTCCTATAATTGTAAACGGTTCCCTTCAATTGGATCTGCTCCCCTCGTACAACA
GAATATGGTATGTTCATCTCCAGGAAGACATCTTTGAACACCTTTGCCTTGAGTGTGTCAGCAACACATATACCATTGTCTGA
GATGCCGATGCCTTGAATTTCCCAGGTCGTCAGTGAGTCAGGCAGTGCAACCTGCAGCTGGTTTCTTTTGGGAACACGATGAA
CTTCCCATAGCCAGCTCTCTGGAAAGTAGCTTCGGATTTCTGCCTTCATCACTGGTAACAGGGCCTTTATTTGGATCCTTCCC
AACAGCATGCCTTTGTGGTGGCTTTCTTTTCGGATCTTATCCGCAATAGTACAACACTCGTTGAAGGCCCTGATGCAGTGTGG
GCCTATGGTCACCCGGGCAACTCGCTGCTCACAGGTTTCGTATTTGTTTTCTCGGGCTCCATCATAACAGCATTTCTTGGGCA
CACGGTGTTTGTATTTAGCAGCTTGTTCTTCCACTTTCTGATGCAGGAGCTGCAGGTCTCTCTTTGGCCTGAGAATTTCCTTA
CAAGAGTCATCGTGGTATTGGGAGTCATCTGCGTTTGCATTGGTGAGGAAGGTGAGCCCAGCTAGATGGAATACATCTACATT
GTCACGGCCACCACCTGCCCCACAGCCCAGGTCACTCTTGTCATCAAAAGCTTGGAACACTCTTTGCATGGCCCTTTTGGCTT
TTCCCCGGACTCCATACACAGCGCTGTCCACCGCAGATAGTGCCACCCATGAGTCTGCTTCAGTCACCATGTCAAGGGACACA
GTTTGGCCTGGAGAATACACGTCTTTATCTGGAGACAGATGGACCTGGAGCTGGTTGCCACACTTCTCCTCAATGTTTATCCA
GACTGCGTCAGCCACCAATTCTGCTGTCTGCTCCCCCGTGACTATGTAATAGACCAGGAGCCGCGCTGAAGGAACCATGTCCT
GTGTCACTGGGATGTTTATATTTTGATAAGATGAATAGAGAAGTTTCTCCTTTGTGCCATACTGTACAATTTTGCCTTTGGAT
AAAATCAAGTAATTATAGTGAGTTATTTTGTCAATATATGGACTCTTGGGGGTGACGATAATATTCAGATATTCTCCCACAAG
CATGGGCTTGTAGTTTTCAGTCCAGCCAATGTAAATGTAACTCTGGCTGAGGGATGAGTATGTAACTGCTTCATATTCTTTGC
TGGCTTGATTTTCTTCGGGAAGTTCCGGGGCATCAGTTTTGACCTCAAACTTCAGTGATGTCACTTCTGATGGGAGGTTCACC
ACAAATGAAGCCACTCCATCAGCAGAGTGTGTGATGCTCCTCTTTGGTTCCAAGTCAGATGTCTCTTGATTCACATTGACTGT
TTGTGCCATCAGAGTTACTGGGACCCCTCCTACCAACTGCTCGAGTGAATCCTTAACCTGTACCTTGATGGAAAATGGAATCC
CAGGCTTCAGGAAAAGAGGGGTAGCGACCAAATTCAGTGTATAGGGAGAGAGGACGTATTTGATGCCAGGAATTTCTGCCTCT
TCCGAAAACCCACCTACAAGGGCAGCAAGCACTGAGTTAATGTTTCCGGAAAGATGATGGCGGGCTGTAGGACATCTGTCGGT
CTCTGTGCTATCCAT

1. A double-stranded ribonucleic acid (dsRNA) agent for use in inhibiting expression of complement component C5 for the prevention or treatment of Alzheimer's disease, atherosclerosis, or inflammation of the choroid plexus (ChP), wherein the dsRNA agent comprises a sense strand and an antisense strand, wherein the nucleotide sequence of the sense strand comprises 5′-UGACAAAAUAACUCACUAUAA-3′ and the nucleotide sequence of the antisense strand comprises 5′-UUAUAGUGAGUUAUUUUGUCAAU-3′.

2. The dsRNA agent of claim 1, wherein the nucleotide sequence of the sense strand consists of 5′-UGACAAAAUAACUCACUAUAA-3′ and the nucleotide sequence of the antisense strand consists of 5′-UUAUAGUGAGUUAUUUUGUCAAUdTdT-3′.

3. The dsRNA agent of claim 1, wherein substantially all of the nucleotides of the sense strand and all of the nucleotides of the antisense strand comprise a modification.

4. The dsRNA agent of claim 3, wherein substantially all of the nucleotides of the sense strand are modified nucleotides selected from the group consisting of a 2′-O-methyl modification, a 2′-fluoro modification, and a 3′-terminal deoxy-thymine (dT) nucleotide.

5. The dsRNA agent of claim 4, wherein all of the nucleotides of the sense strand comprise a modification selected from the group consisting of a 2′-O-methyl modification, a 2′-fluoro modification, and a 3′-terminal deoxy-thymine (dT) nucleotide.

6. The dsRNA agent of claim 3, wherein all of the nucleotides of the antisense strand are modified nucleotides selected from the group consisting of a 2′-O-methyl modification, a 2′-fluoro modification, and a 3′-terminal deoxy-thymine (dT) nucleotide

7. The dsRNA agent of claim 1, wherein the sense strand comprises two phosphorothioate internucleotide linkages at the 5′-terminus, and the antisense strand comprises two phosphorothioate internucleotide linkages at the 5′-terminus and two phosphorothioate internucleotide linkages at the 3′-terminus.

8. The dsRNA agent of claim 1, wherein the sense strand is conjugated to a ligand comprising one or more GalNAc derivatives attached through a branched bivalent or trivalent linker at the 3′-terminus.

9. (canceled)

10. (canceled)

11. (canceled)

12. The dsRNA agent of claim 1, wherein each strand is independently 21-30 nucleotides in length.

13. (canceled)

14. The dsRNA agent of claim 8, wherein the ligand is an N-acetylgalactosamine (GalNAc) derivative.

15. The dsRNA agent of claim 8, wherein the ligand is

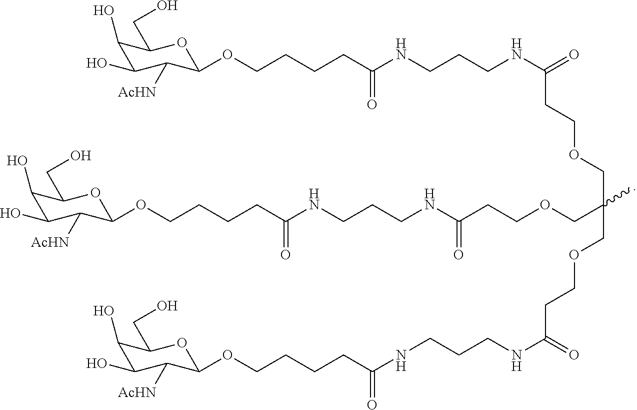 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

16. The dsRNA agent of claim 15, wherein the sense strand is conjugated to the ligand as shown in the following schematic 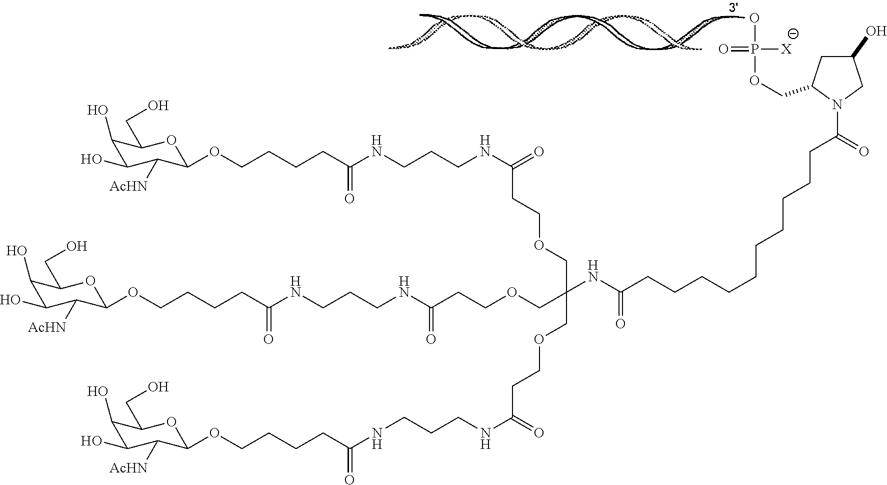
and, wherein X is O or S.

17. The dsRNA agent of claim 16, wherein X is O.

18. A pharmaceutical composition for use in preventing or treating Alzheimer's disease, atherosclerosis, or inflammation of the choroid plexus (ChP), comprising a double-stranded ribonucleic acid (dsRNA) agent which inhibits expression of complement component C5, wherein the dsRNA agent comprises a sense strand and an antisense strand, wherein the nucleotide sequence of the sense strand comprises 5′-UGACAAAAUAACUCACUAUAA-3′ and the nucleotide sequence of the antisense strand comprises 5′-UUAUAGUGAGUUAUUUUGUCAAU-3′.

19. The pharmaceutical composition of claim 18, wherein the pharmaceutical composition is formulated for subcutaneous administration.

20-25. (canceled)

26. A method of preventing or treating Alzheimer's disease, atherosclerosis, or inflammation of the choroid plexus (ChP) in a subject, comprising administering to the subject an effective amount of a double-stranded ribonucleic acid (dsRNA) agent which inhibits expression of complement component C5, wherein the dsRNA agent comprises a sense strand and an antisense strand, wherein the nucleotide sequence of the sense strand comprises 5′-UGACAAAAUAACUCACUAUAA-3′ and the nucleotide sequence of the antisense strand comprises 5′-UUAUAGUGAGUUAUUUUGUCAAU-3′, thereby preventing or treating the subject having Alzheimer's disease, atherosclerosis, or inflammation of the choroid plexus (ChP).

27. The method of claim 26, wherein the dsRNA agent is administered subcutaneously.

28. The method of claim 26, wherein the subject is human.

29-33. (canceled)
